SEQUENCE LISTING (4 sequences)

SEQ ID NO: 1 - protein, 818 aa, Streptococcus pneumoniae
SYELGRHQAGQVKKESNRVSYIDGDQAGQKAENLTPDEVSKREGINAEQIVIKITDQGYVTSHGDHYHYYNGKVPYDAIISEELLMKDPNYQLKDSDIVNEIKGGYVIKVDGKYYVYLKDAAHADNIRTKEEIKRQKQEHSHNHGGGSNDQAVVAARAQGRYTTDDGYIFNASDIIEDTGDAYIVPHGDHYHYIPKNELSASELAAAEAYWNGKQGSRPSSSSSYNANPAQPRLSENHNLTVTPTYHQNQGENISSLLRELYAKPLSERHVESDGLIFDPAQITSRTARGVAVPHGNHYHFIPYEQMSELEKRIARIIPLRYRSNHWVPDSRPEQPSPQSTPEPSPSPQPAPNPQPAPSNPIDEKLVKEAVRKVGDGYVFEENGVSRYIPAKDLSAETAAGIDSKLAKQESLSHKLGAKKTDLPSSDREFYNKAYDLLARIHQDLLDNKGRQVDFEALDNLLERLKDVPSDKVKLVDDILAFLAPIRHPERLGKPNAQITYTDDEIQVAKLAGKYTTEDGYIFDPRDITSDEGDAYVTPHMTHSHWIKKDSLSEAERAAAQAYAKEKGLTPPSTDHQDSGNTEAKGAEAIYNRVKAAKKVPLDRMPYNLQYTVEVKNGSLIIPHYDHYHNIKFEWFDEGLYEAPKGYTLEDLLATVKYYVEHPNERPHSDNGFGNASDHVRKNKVDQDSKPDEDKEHDEVSEPTHPESDEKENHAGLNPSADNLYKPSTDTEETEEEAEDTTDEAEIPQVENSVINAKIADAEALLEKVTDPSIRQNAMETLTGLKSSLLLGTKDNNTISAEVDSLLALLKESQPAPI

SEQ ID NO: 2 - protein, 6 aa, artificial sequence
  S. pneumoniae polyhistidine triad protein (Pht) family histidine triad motif
HXXHXH

SEQ ID NO: 3 - protein, 4 aa, unknown
  S. pneumoniae protein having a Type II Signal sequence Motif
LXXC

SEQ ID NO: 4 - protein, 5 aa, unknown
  S. pneumoniae  surface protein Cell Wall Anchored motif
LPXTG

(19) United States
(12) Patent Application Publication
(10) Pub. No.: US 2014/0105927 A1
(43) Pub. Date: Apr. 17, 2014

(54) IMMUNOGENIC COMPOSITION

(71) Applicant: GLAXOSMITHKLINE BIOLOGICALS SA, Rixensart (BE)

(72) Inventor: VINCENT VERLANT, Rixensart (BE)

(73) Assignee: GlaxoSmithKline Biologicals S.A., Rixensart (BE)

(21) Appl. No.: 13/827,203

(22) Filed: Mar. 14, 2013

Related U.S. Application Data
(60) Provisional application No. 61/714,956, filed on Oct. 17, 2012.

(51) Int. Cl. A61K 39/09

(52) U.S. Cl. CPC A61K 39/092
USPC 424/190.1; 424/244.1; 424/197.11

(57) ABSTRACT

The present invention relates to an immunogenic composition comprising
(i) at least one Streptococcus pneumoniae protein;
(ii) a Streptococcus pneumoniae capsular saccharide derived from a strain of a targeted serotype of Streptococcus pneumoniae;
for use in enhancing antibody-mediated opsonic activity against the targeted serotype of Streptococcus pneumoniae.

This application is a United States 111(a) Application which claims priority from Provisional Application Ser. No. 61/714,956, filed in the United States Oct. 17, 2012, the contents of which is herein incorporated by reference in its entirety.

### TECHNICAL FIELD

The present invention relates to improved immunogenic compositions and vaccines, and their use in medicine. In particular the invention relates to immunogenic compositions for use in enhancing antibody-mediated opsonic activity against a Streptococcus pneumoniae serotype or methods for enhancing antibody-mediated opsonic activity against a Streptococcus pneumoniae serotype.

### BACKGROUND

Streptococcus pneumoniae (S. pneumoniae), also known as the pneumococcus, is a Gram-positive bacterium. S. pneumoniae is a major public health problem all over the world and is responsible for considerable morbidity and mortality, especially among infants, the elderly and immunocompromised persons. S. pneumoniae causes a wide range of important human pathologies including community-acquired pneumonia, acute sinusitis, otitis media, meningitis, bacteremia, septicemia, osteomyelitis, septic arthritis, endocarditis, peritonitis, pericarditis, cellulitis, bronchitis and COPD exacerbations and brain abscess. S. pneumoniae is estimated to be the causal agent in 3,000 cases of meningitis, 50,000 cases of bacteremia, 500,000 cases of pneumonia, and 7,000,000 cases of otitis media annually in the United States alone (Reichler, M. R. et al., 1992, J. Infect. Dis. 166: 1346; Stool, S. E. and Field, M. J., 1989 Pediatr. Infect. Dis J. 8: S11). Mortality rates due to pneumococcal disease are especially high in children younger than 5 years of age from both developed and developing countries. The elderly, the immunocompromised and patients with other underlying conditions (diabetes, asthma) are also particularly susceptible to disease.

The major clinical syndromes caused by S. pneumoniae are widely recognized and discussed in all standard medical textbooks (Fedson D S, Muscher D M. In: Plotkin S A, Orenstein W A, editors. Vaccines. 4th edition. Philadelphia WB Saunders Co, 2004a: 529-588). For instance, Invasive pneumococcal disease (IPD) is defined as any infection in which S. pneumoniae is isolated from the blood or another normally sterile site (Musher D M. Streptococcus pneumoniae. In Mandell G L, Bennett J E, Dolin R (eds). Principles and Practice of Infectious diseases (5th ed). New York, Churchill Livingstone, 2001, p 2128-2147).

Chronic obstructive pulmonary disease (COPD) is a chronic inflammatory disease of the lungs and a major cause of morbidity and mortality worldwide. Approximately one in 20 deaths in 2005 in the US had COPD as the underlying cause. (Drugs and Aging 26:985-999 (2009)). It is projected that in 2020 COPD will rise to the fifth leading cause of disability adjusted life years, chronic diseases, and to the third most important cause of mortality (Lancet 349:1498-1504 (1997)).

Exacerbations of COPD increase rates of hospitalization and mortality and decrease quality of life. Exacerbations are marked by an increase from baseline in dyspnoea, sputum volume and sputum purulence. Approximately 50% of acute exacerbations of symptoms in COPD are caused by non-typeable Haemophilus influenzae, Moraxella catarrhalis, Streptococcus pneumoniae and Pseudomonas aeruginosa.

Although the advent of antimicrobial drugs has reduced the overall mortality from pneumococcal disease, the emergence of antibiotic resistant strains of S. pneumoniae is a serious and rapidly increasing problem. It is therefore important for effective vaccines against S. pneumoniae to be developed. Effective pneumococcal vaccines could have a major impact on the morbidity and mortality associated with S. pneumoniae disease.

The present invention relates to immunogenic compositions for use in enhancing antibody-mediated opsonic activity against a Streptococcus pneumoniae serotype or methods for enhancing antibody-mediated opsonic activity against a Streptococcus pneumoniae serotype. The inventors have found that Pneumococcal proteins can enhance the antibody-mediated opsonic activity against a serotype of Streptococcus pneumoniae when the pneumococcal protein(s) is co-administered in combination with a Streptococcus pneumoniae capsular saccharide derived from a strain of that serotype.

Although a positive interaction between Pneumococcal saccharides and proteins has been demonstrated previously (for example WO02/22167) WO02/22167 refers to a results obtained by simultaneously stimulating the cell mediated branch of the immune system and the humoral branch of the immune system. The effect in WO02/22167 would not enhance the antibody-mediated opsonic activity against a strain of Streptococcus pneumoniae, and would not be reflected in enhanced antibody-mediated opsonic activity measured using an opsonophagocytosis (OPA) assay. This is because the opsonic activity measured using an OPA assay (such as that described in example 2) is measured in the absence of components of the cell mediated immune system.

On the other hand the present inventors have demonstrated, that when humans are immunised with a combination of Pneumococcal saccharides and Pneumococal proteins the antibody mediated opsonic activity against the serotype from which the Pneumococcal saccharide is derived tends to increase (is enhanced), this is measured using an opsonophagocytosis (OPA) assay. This is particularly surprising since Pneumococcal proteins induce little or no antibody mediated opsonic activity when they are injected alone (in the absence of pneumococcal saccharides).

### BRIEF SUMMARY

The inventors have found that an immunogenic composition comprising a Streptococcus pneumoniae protein can enhance the antibody-mediated opsonic activity against a targeted serotype of Streptococcus pneumoniae when the Streptococcus pneumoniae protein is co-administered with a Streptococcus pneumoniae saccharide derived from a strain of the targeted serotype of Streptococcus pneumoniae.

Accordingly in a first aspect there is provided immunogenic composition comprising

(i) at least one Streptococcus pneumoniae protein;
(ii) a Streptococcus pneumoniae capsular saccharide derived from a strain of a targeted serotype of Streptococcus pneumoniae;
for use in enhancing antibody-mediated opsonic activity against the targeted serotype of Streptococcus pneumoniae.

In a second aspect there is provided an immunogenic composition comprising

(i) at least one Streptococcus pneumoniae protein; and
(ii) a Streptococcus pneumoniae capsular saccharide derived from a strain of a targeted serotype of Streptococcus pneumoniae;
for use in immunising a subject so as to enhance antibody-mediated opsonic activity against the targeted serotype of Streptococcus pneumoniae.

In a third aspect there is provided a use of an immunogenic composition comprising

(i) at least one Streptococcus pneumoniae protein; and
(ii) a Streptococcus pneumoniae capsular saccharide derived from a strain of a targeted serotype of Streptococcus pneumoniae;
in enhancing antibody-mediated opsonic activity against the targeted serotype of Streptococcus pneumoniae.

In a fourth aspect there is provided a use of an immunogenic composition comprising

(i) at least one Streptococcus pneumoniae protein; and
(ii) a Streptococcus pneumoniae capsular saccharide derived from a strain of a targeted serotype of Streptococcus pneumoniae;
for immunising a subject so as to enhance antibody-mediated opsonic activity against the targeted serotype of Streptococcus pneumoniae.

In a fifth aspect there is provided immunogenic composition comprising

(i) at least one Streptococcus pneumoniae protein; and
(ii) a Streptococcus pneumoniae capsular saccharide derived from a strain of a targeted serotype of Streptococcus pneumoniae;
for use in the treatment or prevention of Streptococcus pneumoniae disease in a subject wherein the Streptococcus pneumoniae protein enhances antibody-mediated opsonic activity against the targeted serotype of Streptococcus pneumoniae.

In a sixth aspect there is provided a use of an immunogenic composition comprising

(i) at least one Streptococcus pneumoniae protein; and
(ii) a Streptococcus pneumoniae capsular saccharide derived from a strain of a targeted serotype of Streptococcus pneumoniae;
in the manufacture of a medicament for enhancing antibody-mediated opsonic activity against the targeted serotype of Streptococcus pneumoniae.

In a seventh aspect there is provided a use of an immunogenic composition comprising

(i) at least one Streptococcus pneumoniae protein; and
(ii) a Streptococcus pneumoniae capsular saccharide derived from a strain of a targeted serotype of Streptococcus pneumoniae;
in the manufacture of a medicament for use in the treatment or prevention of Streptococcus pneumoniae disease wherein upon immunisation, the at least one Streptococcus pneumoniae protein enhances antibody-mediated opsonic activity against the targeted serotype of Streptococcus pneumoniae.

In an eighth aspect there is provided a method for enhancing antibody-mediated opsonic activity against a targeted serotype of Streptococcus pneumoniae in a subject the method comprising:

co-administering the (i) at least one Streptococcus pneumoniae protein; and (ii) a Streptococcus pneumoniae capsular saccharide derived from a strain of the targeted serotype of Streptococcus pneumoniae; wherein co-administering the Streptococcus pneumoniae capsular saccharide in combination with the at least one Streptococcus pneumoniae protein enhances antibody-mediated opsonic activity against the targeted serotype of Streptococcus pneumoniae; wherein co-administering is performed by administering an immunogenic composition comprising the Streptococcus pneumoniae capsular saccharide in combination with the at least one Streptococcus pneumoniae protein.

In a ninth aspect there is provided method of immunising a human host against diseases caused by Streptococcus pneumoniae infection comprising administering to the host an immunoprotective dose of an immunogenic composition comprising (i) at least one Streptococcus pneumoniae protein; and (ii) a Streptococcus pneumoniae capsular saccharide derived from a strain of a targeted serotype of Streptococcus pneumoniae; wherein the at least one Streptococcus pneumoniae protein enhances antibody-mediated opsonic activity against the targeted serotype of Streptococcus pneumoniae.

In a tenth aspect there is provided method of increasing an immune response in a subject against infection or disease caused by Streptococcus pneumoniae, the method comprising:

co-administering (i) at least one Streptococcus pneumoniae protein; and (ii) a Streptococcus pneumoniae capsular saccharide derived from a strain of a targeted serotype of Streptococcus pneumoniae as an immunogenic composition, wherein co-administering the Streptococcus pneumoniae capsular saccharide in combination with the at least one Streptococcus pneumoniae protein enhances antibody-mediated opsonic activity against the targeted serotype of Streptococcus pneumoniae or

wherein an increased immune response is measurable as an enhanced antibody-mediated opsonic activity against the targeted serotype of Streptococcus pneumoniae.

In an eleventh aspect there is provided a method of increasing an immune response in a subject against infection or disease caused by Streptococcus pneumoniae, the method comprising:

providing at least one Streptococcus pneumoniae protein;

formulating the at least one Streptococcus pneumoniae protein in combination with a Streptococcus pneumoniae capsular saccharide derived from a strain of a targeted serotype of Streptococcus pneumoniae to produce an immunogenic composition; and administering to a subject at risk of infection with Streptococcus pneumoniae one or more doses of the immunogenic composition, thereby eliciting an immune response, which immune response is measurable as an enhanced antibody-mediated opsonic activity against the targeted serotype of Streptococcus pneumoniae.

In a twelfth aspect there is provided an immunogenic composition for enhancing antibody-mediated opsonic activity against a targeted serotype of Streptococcus pneumoniae comprising

(i) at least one Streptococcus pneumoniae protein;
(ii) a Streptococcus pneumoniae capsular saccharide derived from a strain of the targeted serotype of Streptococcus pneumoniae.

### BRIEF DESCRIPTION OF THE DRAWINGS

FIG. 1: Bar chart showing anti-dPly antibody titres, according to an ELISA assay, in human adults after immunisation with 10 μg detoxified Pneumolysin (Ply-10), 30 μg detoxified pneumolysin (Ply-30), 10 μg of detoxified pneumolysin and 10 μg of PhtD (PIPh-10), 30 μg of detoxified pneumolysin and 30 μg of PhtD (PIPh-3), a vaccine comprising 10 pneumococcal conjugates combined with 10 μg detoxified pneumolysin and 10 μg of PhtD (10vPP10), a vaccine comprising 10 pneumococcal conjugates combined with 30 μg detoxified pneumolysin and 30 μg of PhtD (10vPP30), and a vaccine comprising 23 unconjugated pneumococcal saccharides. The white bars represent immunogenicity prior to immunisation, the grey bars represent immunogenicity after 1 dose of vaccine and the black bars represent immunogenicity after 2 doses of vaccine.

FIG. 2: Similar bar chart to FIG. 2 but depicting the anti-PhtD titres rather than anti-Ply titres.

FIG. 3: Bar chart showing anti-PS1 opsonic antibody titres, according to an opsonophagocytosis assay, in human adults after immunisation with 10 μg detoxified Pneumolysin (Ply-10), 30 μg detoxified pneumolysin (Ply-30), 10 μg of detoxified pneumolysin and 10 μg of PhtD (PIPh-10), 30 μg of detoxified pneumolysin and 30 μg of PhtD (PIPh-3), a vaccine comprising 10 pneumococcal conjugates combined with 10 μg detoxified pneumolysin and 10 μg of PhtD (10vPP10), a vaccine comprising 10 pneumococcal conjugates combined with 30 μg detoxified pneumolysin and 30 μg of PhtD (10vPP30), and a vaccine comprising 23 unconjugated pneumococcal saccharides. The white bars represent immunogenicity prior to immunisation, the grey bars represent immunogenicity after 1 dose of vaccine and the black bars represent immunogenicity after 2 doses of vaccine.

FIG. 4: Similar bar chart to FIG. 3 but depicting the anti-PS4 titres rather than the anti-PS1 titers.

FIG. 5: Similar bar chart to FIG. 3 but depicting the anti-PS5 titres rather than the anti-PS1 titers.

FIG. 6: Similar bar chart to FIG. 3 but depicting the anti-PS6B titres rather than the anti-PS1 titers.

FIG. 7: Similar bar chart to FIG. 3 but depicting the anti-PS7F titres rather than the anti-PS1 titers.

FIG. 8: Similar bar chart to FIG. 3 but depicting the anti-PS9V titres rather than the anti-PS1 titers.

FIG. 9: Similar bar chart to FIG. 3 but depicting the anti-PS14 titres rather than the anti-PS1 titers.

FIG. 10: Similar bar chart to FIG. 3 but depicting the anti-PS18C titres rather than the anti-PS1 titers.

FIG. 11: Similar bar chart to FIG. 3 but depicting the anti-PS19F titres rather than the anti-PS1 titers.

FIG. 12: Similar bar chart to FIG. 3 but depicting the anti-PS23F titres rather than the anti-PS1 titers.

FIG. 13: Sequence listing

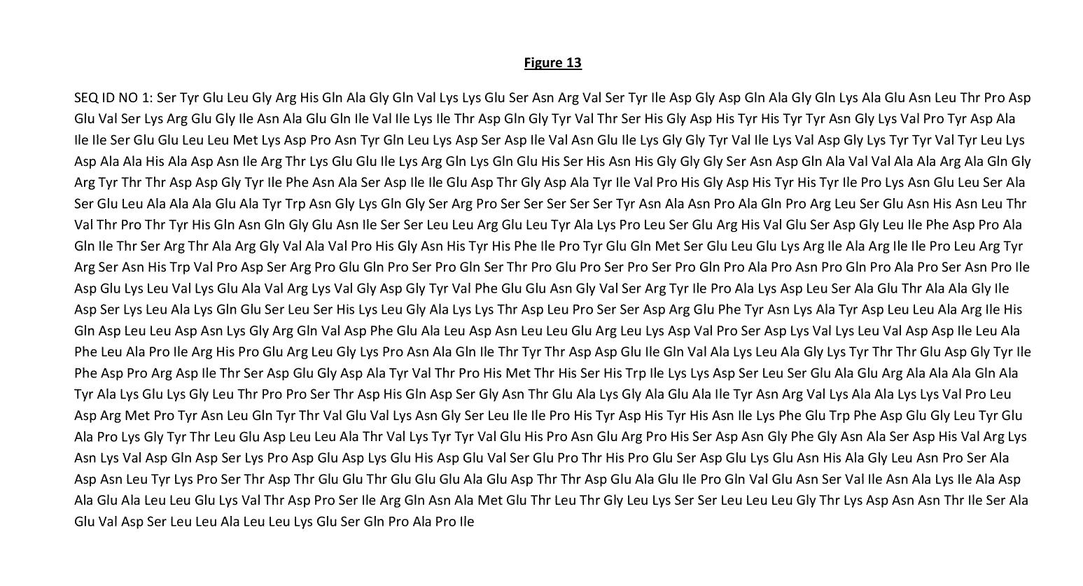

FIG. 14: Graph comparing the immunogenicity of a composition comprising 12 saccharide conjugates, PhtD, dPly and PE-PilA (12V+prot) with a composition comprising 12 saccharides conjugates (12V) and a composition comprising 10 saccharide conjugates (10V) as measured after injection of mice using an anti-saccharide ELISA assay. GMC=geometric mean concentration. IC=confidence intervals.

FIG. 15: Graph comparing the immunogenicity of a composition comprising 12 saccharide conjugates, PhtD, dPly and PE-PilA (12V+prot) with a composition comprising 12 saccharides conjugates (12V) and a composition comprising 10 saccharide conjugates (10V) as measured after injection of mice using an opsonophagocytosis assay. GMT=geometric means titer.

FIG. 16: Graph comparing the immunogenicity of a composition comprising 12 saccharide conjugates, PhtD, dPly and PE-PilA (12V+prot) with a composition comprising PhtD, dPly and PE-PilA alone (prot) as measured after injection of mice using an anti-protein ELISA assay. GMC=geometric mean concentration. IC=confidence intervals.

FIG. 17: Graph comparing the immunogenicity of a composition comprising 12 saccharide conjugates, PhtD, dPly and PE-PilA (12V+prot) with a composition comprising 12 saccharides conjugates (12V) and a composition comprising 10 saccharide conjugates (10V) as measured after injection of guinea pigs using an opsonophagocytosis assay. GMT=geometric means titer.

FIG. 18: Graph comparing the immunogenic of a composition comprising 12 saccharide conjugates, PhtD, dPly and PE-PilA (12V+prot) with a composition comprising 12 saccharides conjugates (12V) and a composition comprising 10 saccharide conjugates (10V) as measured after injection of guinea pigs using an anti-saccharide ELISA. GMC=geometric mean concentration. IC=confidence intervals.

FIG. 19: Graph comparing the immunogenic of a composition comprising 12 saccharide conjugates, PhtD, dPly and PE-PilA (12V+prot) with a composition comprising PhtD, dPly and PE-PilA alone (prot) as measured after injection of guinea pigs using an anti-protein ELISA. GMC=geometric mean concentration. ic=confidence intervals.

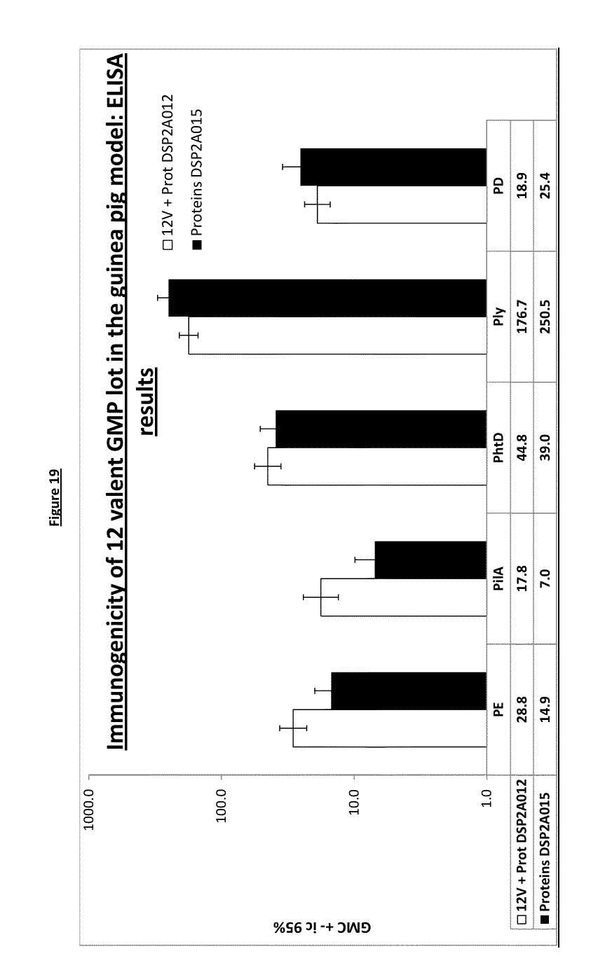

### DETAILED DESCRIPTION

Opsonic antibodies have been shown to be a critical protection mechanism induced by saccharide-based pneumococcal vaccines. Opsonophagocytosis assays (OPA assays) have been developed and validated to characterize and assess current and possible future pneumococcal vaccines (Validation of a routine opsonophagocytosis assay to predict invasive pneumococcal disease efficacy of conjugate vaccine in children; Henckaerts et al; Vaccine 25 (2007):2518-2527). The inventors have surprisingly found that when pneumococcal proteins are administered together with pneumococcal saccharides, the antibody mediated opsonic activity measured by OPA assay against Streptococcus pneumoniae strains of the same serotype as the pneumococcal saccharides appears enhanced.

As the skilled person will appreciate opsonophagocytosis assays involve incubating serum obtained from a subject together with opsonophagocytic cells, a source of complement, and bacteria of the targeted Streptococcus pneumoniae serotype. The components of the sera (in general the functional antibodies present) promote opsonophagocytosis of the bacteria and the extent to which these components promote opsonophagocytosis can be measured by determining the number of remaining (not ‘killed’) bacteria at the end of the incubation. This is a measure of the opsonic activity of the sera and thus the antibodies raised. Antibodies which can bind to the bacteria promote opsonophagocytic ‘killing’ of the bacteria, for example, by binding of the Fc regions of the antibodies to Fc receptors on the surface of phagocytic cells enabling the phagocytic cell to engulf bacteria coated with antibodies.

The present inventors observed immunizing human subjects with a vaccine comprising pneumococcal saccharides and a higher dose of pneumococcal proteins trends towards a higher opsonic activity against the serotypes from which the saccharides are derived compared to immunisation of subjects with a vaccine comprising pneumococcal saccharides and a lower dose of pneumococcal proteins. This cannot be due to the presence of humoral immunity since the in vitro OPA assay occurs in the absence of the humoral immune system. In the context of the invention the terms ‘higher dose’ and ‘lower dose’ can be considered to be relative terms, in the examples of the present application the inventors have demonstrated that the antibody-mediated opsonic activity of a composition comprising 10 saccharides is higher when the saccharides are administered with pneumococcal proteins at doses of 30 μg than when the saccharides are administered with pneumococcal proteins at dose of 10 μg, however a similar effect could be seen if the saccharides were administered with different doses of the proteins, what is important is that increasing the dose of pneumococcal proteins leads to higher antibody-mediated opsonic activity. However in one embodiment the term ‘higher dose’ can be considered to refer to a dose of 30 μg and the term ‘lower dose’ can be considered to refer to a dose of 10 μg.

Without being bound by any theory, this effect could be explained, for example, by the pneumococcal protein having some kind of ‘adjuvant’ effect on the pneumococcal saccharides, for example the pneumococcal protein may have a stabilising effect on a key epitope found within the saccharides. Alternatively it may be that pneumococcal proteins elicit a small level of opsonic activity that is not normally significant (and thus has not been previously recognized), but when the proteins are administered in the presence of pneumococcal saccharides the opsonic activity of the proteins is added to the opsonic activity to the saccharides leading to a statistically significant opsonic activity and greater immunity.

In addition the inventors have found that in some cases this effect can be used to overcome antigen suppression. For example in some cases it may be that the opsonic activity against the saccharides is reduced when a small dose of pneumococcal protein is coadministered due to the antigen suppression effect, whilst co-administration of the saccharides with a higher dose of the pneumococcal proteins raises the overall level of opsonic activity against the targeted serotype back to the level of response when the saccharide is administered in the absence of a pneumococcal protein.

Accordingly in a first aspect, there is provided an immunogenic composition comprising

(i) at least one Streptococcus pneumoniae protein;
(ii) a Streptococcus pneumoniae capsular saccharide derived from a strain of a targeted serotype of Streptococcus pneumoniae;
for use in enhancing antibody-mediated opsonic activity against the targeted serotype of Streptococcus pneumoniae.

The term ‘targeted serotype’ refers to the serotype from which the Streptococcus pneumoniae capsular saccharide is derived optionally this includes serotypes which are cross-reactive with the serotype from which the Streptococcus pneumoniae capsular saccharide is derived. For example if the Streptococcus pneumoniae saccharide is a serotype 19F capsular saccharide, the targeted serotype is a serotype 19F Streptococcus pneumoniae strain or may be a serotype 19A Streptococcus pneumoniae strain (as some serotype 19A capsular saccharide conjugates can provide protection against 19F disease, i.e. 19F saccharide conjugates are cross-reactive).

The term ‘capsular saccharide derived from a strain of a targeted serotype of Streptococcus pneumoniae’ refers to a polysaccharide or oligosaccharide which can be isolated from the capsule of the targeted serotype of Streptococcus pneumoniae. For example the following saccharide structures have been isolated from serotypes of Streptococcus pneumoniae:

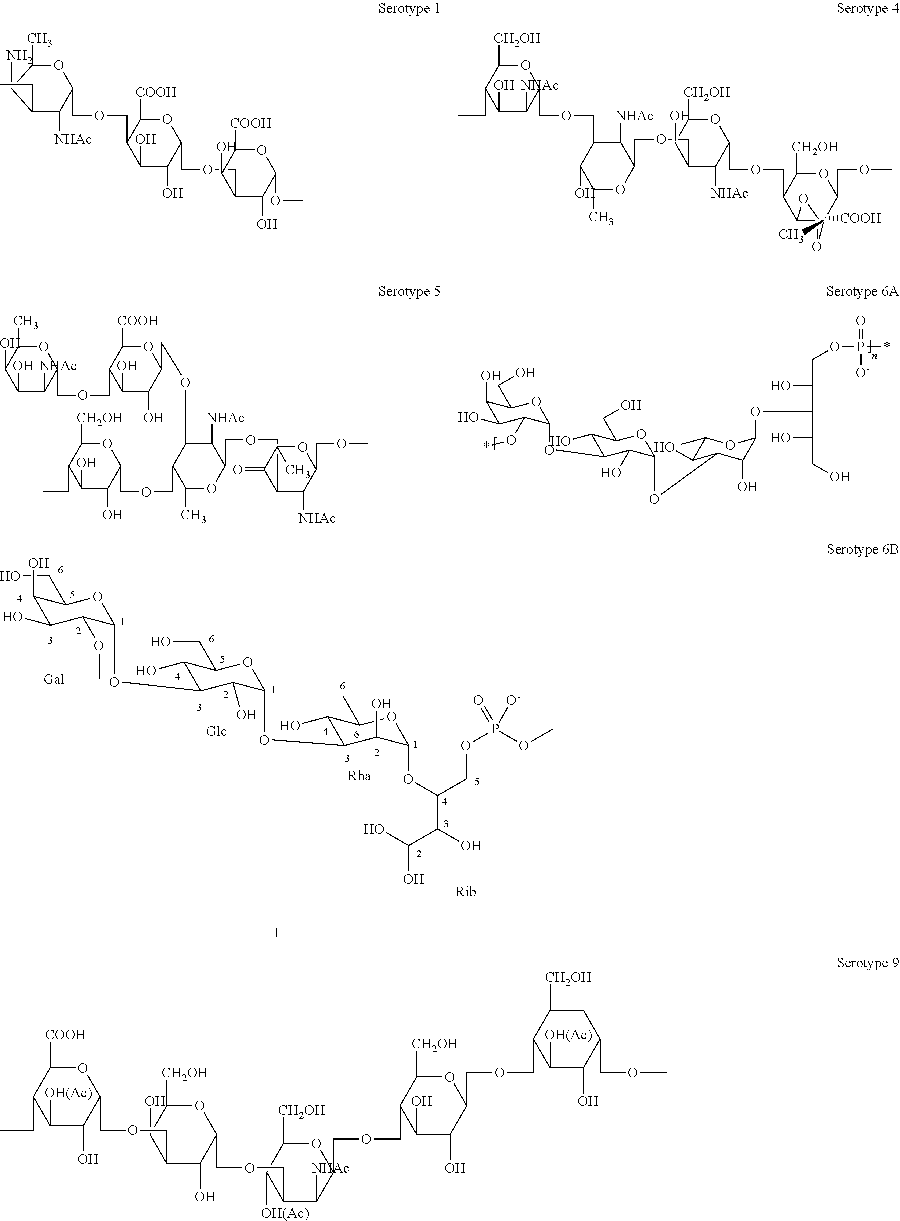

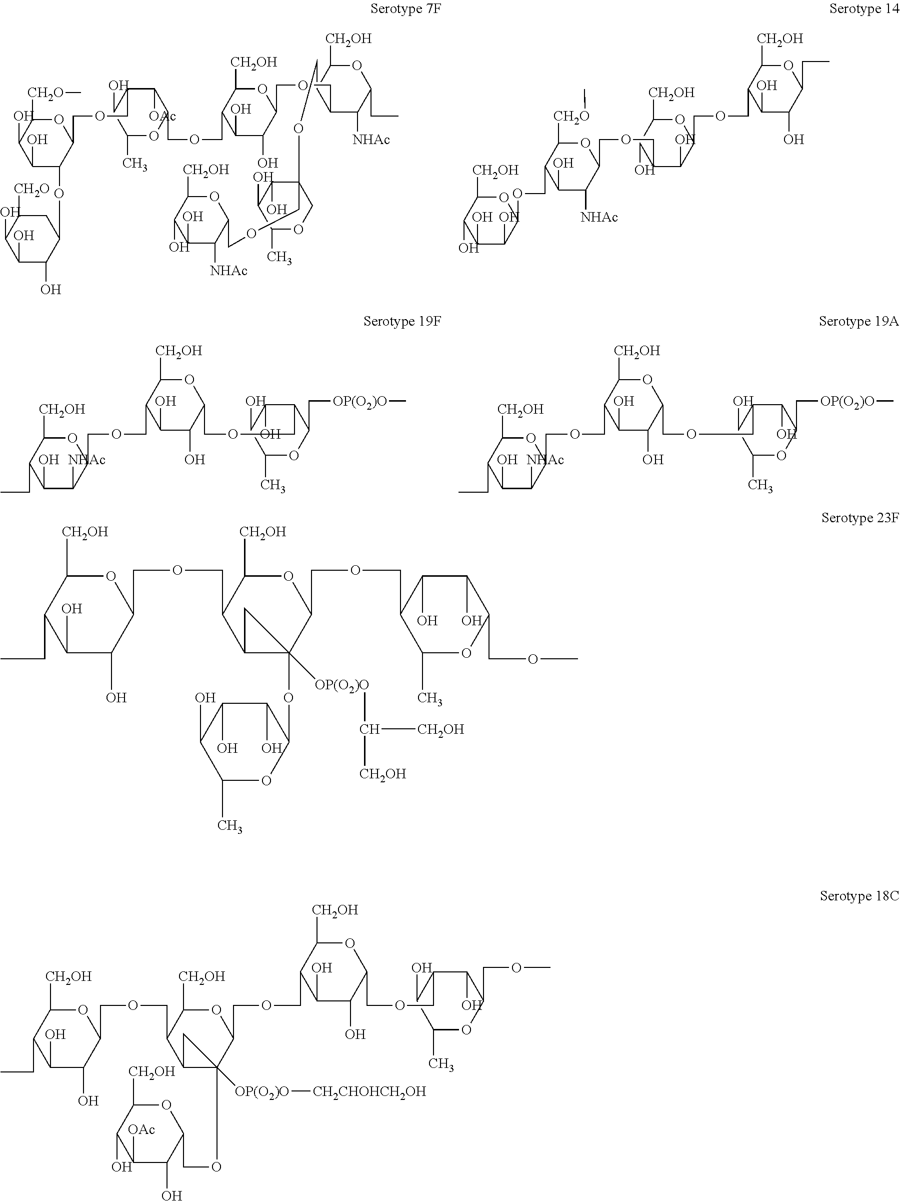

The term ‘antibody mediated opsonic activity’ refers to opsonic activity as measured using an in vitro opsonophagocytosis (OPA) assay (as described above). Accordingly the term ‘enhancing antibody-mediated opsonic activity’ means that the immunogenic composition of the invention raises the antibody-mediated opsonic activity. An enhancement in the antibody mediated opsonic activity against the targeted strain can be measured by immunizing a subject such as a human with a Streptococcus pneumoniae saccharide and a Streptococcus pneumoniae protein, taking a sample of the serum from the subject, measuring the opsonic activity with an OPA assay using bacteria from the targeted serotype and comparing this with the opsonic activity of a reference serum sample. A reference serum sample is a sample of serum obtained by immunizing a subject with an equivalent dose of the Streptococcus pneumoniae saccharide either alone, or in the presence of a lower dose of the at least one Streptococcus pneumoniae protein. If the opsonic activity measured is higher for the sample serum than for the reference serum the antibody mediated opsonic activity against the targeted strain is enhanced.

The antibody mediated opsonic activity against a targeted serotype can be measured using an opsonophagocytosis assay such as that described in example 2.

In one embodiment the antibody-mediated opsonic activity against a targeted serotype after immunisation using an immunogenic composition of the invention is greater than 1.1, 1.2, 1.3, 1.4, 1.5, 1.6, 1.7, 1.8, 1.9, 2, 2.1 or 2.5 times higher than the antibody-mediated opsonic activity against the targeted serotype of the reference sample. The term ‘equivalent dose’ means a dose which is the same or within 10% of the reference dose.

In one embodiment the antibody-mediated opsonic activity against a targeted serotype is the same when the protein is present and the protein is absent, and yet the level of antibodies raised is higher when the Streptococcus pneumoniae saccharide is administered in combination with a higher dose of the Streptococcus pneumoniae protein compared with the level of antibodies raised when the Streptococcus pneumoniae saccharide is administered in combination with a lower dose of the Streptococcus pneumoniae protein. This could occur where the ‘enhancing’ effect of the Streptocococcus pneumoniae protein is counteracted by an antigen interference effect, i.e. adding further antigens to an existing vaccine composition may lead to a general reduction in immunogenicity.

In a second aspect there is provided an immunogenic composition comprising

(i) at least one Streptococcus pneumoniae protein; and
(ii) a Streptococcus pneumoniae capsular saccharide derived from a strain of a targeted serotype of Streptococcus pneumoniae;
for use in immunising a subject so as to enhance antibody-mediated opsonic activity against the targeted serotype of Streptococcus pneumoniae.

The phrase ‘so as to enhance antibody-mediated opsonic activity’ should be interpreted in line with the definition of ‘enhancing antibody-mediated opsonic activity’ presented in paragraph [031]

In a third aspect there is provided a use of an immunogenic composition comprising

(i) at least one Streptococcus pneumoniae protein; and
(ii) a Streptococcus pneumoniae capsular saccharide derived from a strain of a targeted serotype of Streptococcus pneumoniae;
in enhancing antibody-mediated opsonic activity against the targeted serotype of Streptococcus pneumoniae.

In a fourth aspect there is provided a use of an immunogenic composition comprising

(i) at least one Streptococcus pneumoniae protein; and
(ii) a Streptococcus pneumoniae capsular saccharide derived from a strain of a targeted serotype of Streptococcus pneumoniae;
for immunising a subject so as to enhance antibody-mediated opsonic activity against the targeted serotype of Streptococcus pneumoniae.

In a fifth aspect there is provided an immunogenic composition comprising

(i) at least one Streptococcus pneumoniae protein; and
(ii) a Streptococcus pneumoniae capsular saccharide derived from a strain of a targeted serotype of Streptococcus pneumoniae;
for use in the treatment or prevention of Streptococcus pneumoniae disease in a subject wherein the Streptococcus pneumoniae protein enhances antibody-mediated opsonic activity against the targeted serotype of Streptococcus pneumoniae.

The term ‘enhances antibody-mediated opsonic activity’ should be interpreted in line with the definition of ‘enhancing antibody-mediated opsonic activity’ presented in paragraph [031].

In a sixth aspect there is provided a use of an immunogenic composition comprising

(i) at least one Streptococcus pneumoniae protein; and
(ii) a Streptococcus pneumoniae capsular saccharide derived from a strain of a targeted serotype of Streptococcus pneumoniae;
in the manufacture of a medicament for enhancing antibody-mediated opsonic activity against the targeted serotype of Streptococcus pneumoniae.

In a seventh aspect there is provided a use of an immunogenic composition comprising

(i) at least one Streptococcus pneumoniae protein; and
(ii) a Streptococcus pneumoniae capsular saccharide derived from a strain of a targeted serotype of Streptococcus pneumoniae;
in the manufacture of a medicament for use in the treatment or prevention of Streptococcus pneumoniae disease wherein upon immunisation, the at least one Streptococcus pneumoniae protein enhances antibody-mediated opsonic activity against the targeted serotype of Streptococcus pneumoniae.

In an eighth aspect there is provided a method for enhancing antibody-mediated opsonic activity against a targeted serotype of Streptococcus pneumoniae in a subject the method comprising:

co-administering (i) at least one Streptococcus pneumoniae protein; and (ii) a Streptococcus pneumoniae capsular saccharide derived from a strain of the targeted serotype of Streptococcus pneumoniae; wherein co-administering the Streptococcus pneumoniae capsular saccharide in combination with the at least one Streptococcus pneumoniae protein enhances antibody-mediated opsonic activity against the targeted serotype of Streptococcus pneumoniae; wherein co-administering is performed by administering an immunogenic composition comprising the Streptococcus pneumoniae capsular saccharide in combination with the at least one Streptococcus pneumoniae protein.

In a ninth aspect there is provided a method of immunising a human host against diseases caused by Streptococcus pneumoniae infection comprising administering to the host an immunoprotective dose of an immunogenic composition comprising (i) at least one Streptococcus pneumoniae protein; and (ii) a Streptococcus pneumoniae capsular saccharide derived from a strain of a targeted serotype of Streptococcus pneumoniae; wherein the at least one Streptococcus pneumoniae protein enhances antibody-mediated opsonic activity against the targeted serotype of Streptococcus pneumoniae.

In a tenth aspect there is provided a method of increasing an immune response in a subject against infection or disease caused by Streptococcus pneumoniae, the method comprising:

co-administering (i) at least one Streptococcus pneumoniae protein; and (ii) a Streptococcus pneumoniae capsular saccharide derived from a strain of a targeted serotype of Streptococcus pneumoniae as an immunogenic composition, wherein co-administering the Streptococcus pneumoniae capsular saccharide in combination with the at least one Streptococcus pneumoniae protein enhances antibody-mediated opsonic activity against the targeted serotype of Streptococcus pneumoniae or

wherein an increased immune response is measurable as an enhanced antibody-mediated opsonic activity against the targeted serotype of Streptococcus pneumoniae.

In an eleventh aspect there is provided method of increasing an immune response in a subject against infection or disease caused by Streptococcus pneumoniae, the method comprising:

providing at least one Streptococcus pneumoniae protein;

formulating the at least one Streptococcus pneumoniae protein in combination with a Streptococcus pneumoniae capsular saccharide derived from a strain of a targeted serotype of Streptococcus pneumoniae to produce an immunogenic composition; and administering to a subject at risk of infection with Streptococcus pneumoniae one or more doses of the immunogenic composition, thereby eliciting an immune response, which immune response is measurable as an enhanced antibody-mediated opsonic activity against the targeted serotype of Streptococcus pneumoniae.

In a twelfth aspect there is provided an immunogenic composition for enhancing antibody-mediated opsonic activity against a targeted serotype of Streptococcus pneumoniae comprising

(i) at least one Streptococcus pneumoniae protein;
(ii) a Streptococcus pneumoniae capsular saccharide derived from a strain of the targeted serotype of Streptococcus pneumoniae.

The term ‘co-administering’ or ‘co-administration’ refers to administering a dose of two or more components on the same day, this may be carried out by combining the components into a single vaccine, for example in a single syringe, alternatively this could be achieved by administering one or more components in a first composition and one or more components in a second composition on the same day (for example in different limbs of a human subject). Similarly the term ‘immunogenic composition’ may refer to a kit in which one or more components are present in a first container (such as a vial or syringe) and one or more components are present in a second container, for example the kit may contain a composition comprising the pneumococcal saccharides in a first container and the pneumococcal proteins in a second container.

In one embodiment of the immunogenic composition, use or method of the invention, the antibody-mediated opsonic activity against the targeted strain of Streptococcus pneumoniae is enhanced if the antibody-mediated opsonic activity against the targeted serotype of Streptococcus pneumoniae is greater when the Streptococcus pneumoniae capsular saccharide is co-administered with the Streptococcus pneumoniae protein than antibody-mediated opsonic activity elicited by administering an equivalent dose of the Streptococcus pneumoniae capsular saccharide alone (in the absence of the at least one Streptococcus pneumoniae protein).

In a further embodiment of the immunogenic composition, use or method of the invention, the antibody-mediated opsonic activity against the targeted serotype of Streptococcus pneumoniae is enhanced if the antibody-mediated opsonic activity against the targeted strain of Streptococcus pneumoniae is greater than the antibody-mediated opsonic activity elicited by administering an equivalent dose of the Streptococcus pneumoniae capsular saccharide together with a lower dose of the at least one Streptococcus pneumoniae protein.

### Pneumolysin

In one embodiment the at least one Streptococcus pneumoniae protein comprises detoxified pneumolysin (dPly).

By pneumolysin, or “Ply”, it is meant: native or wild-type pneumolysin from pneumococcus, recombinant pneumolysin, and fragments and/or variants thereof. In an embodiment, pneumolysin is native or wild-type pneumolysin from pneumococcus or recombinant pneumolysin. Ply is a 53 kDa pore-forming toxin and as such has lytic effects on many mammalian cell types, and at sublytic concentrations Ply has numerous other effects, including complement activation in the absence of antipneumolysin antibodies and induction of pro-inflammatory mediators (Hirst et al., 2000; Mitchell & Andrew, 1997; Paton et al., 1984; Zysk et al., 2001). Complement activation is independent of the haemolytic activity of the toxin (Berry et al., 1995). Pneumolysin is now known to interact directly with Toll-like receptor 4 (TLR-4) and known to signal via myeloid differentiation marker 88 to induce production of tumour necrosis factor alpha and interleukin 6 (Malley et al., 2003; Trzcinski et al., 2008). Pneumolysin is produced by all known clinical isolates of S. pneumoniae regardless of serotype and genotype. Expression and cloning of wild-type or native pneumolysin is known in the art. See, for example, Walker et al. (Infect Immun, 55:1184-1189 (1987)), Mitchell et al. (Biochim Biophys Acta, 1007:67-72 (1989) and Mitchell et al (NAR, 18:4010 (1990)). WO2010/071986 describes wild-type Ply, e.g. SEQ IDs 2-42 (for example SEQ IDs 34, 35, 36, 37, 41). In one aspect, pneumolysin is Seq ID No. 34 of WO2010/071986. In another aspect, pneumolysin is Seq ID No. 35 of WO2010/071986. In another aspect, pneumolysin is Seq ID No. 36 of WO2010/071986. In another aspect, pneumolysin is Seq ID No. 37 of WO2010/071986. In another aspect, pneumolysin is Seq ID No. 41 of WO2010/071986. Furthermore, EP1601689B1 describes methods for purifying bacterial cytolysins such as pneumococcal pneumolysin by chromatography in the presence of detergent and high salt.

In an embodiment, the term ‘pneumolysin’ includes fragments and/or variants of pneumolysin, having differences in nucleic acid or amino acid sequences as compared to a wild type sequence. Where fragments of pneumolysin are used, these fragments will be at least about 15, at least about 20, at least about 40, or at least about 60 contiguous amino acid residues in length. In an embodiment of the invention, immunogenic fragments of pneumolysin comprise at least about 15, at least about 20, at least about 40, or at least about 60 contiguous amino acid residues of the full length sequence, wherein said polypeptide is capable of eliciting an immune response specific for said amino acid sequence. Pneumolysin is known to consist of four major structural domains (Rossjohn et al. Cell. 1997 May 30; 89(5):685-92). These domains may be modified by removing and/or modifying one or more of these domains. In an embodiment, the or each fragment contains exactly or at least 1, 2 or 3 domains. In another embodiment, the or each fragment contains exactly or at least 2 or 3 domains. In another embodiment, the or each fragment contains at least 3 domains. The or each fragment may be more than 50, 60, 70, 80, 90, 95, 98, 99 or 100% identical to a wild type pneumolysin sequence.

In accordance with the present invention, a variant of pneumolysin includes sequences in which one or more amino acids are substituted and/or deleted and/or inserted compared to the wild type sequence. Amino acid substitution may be conservative or non-conservative. In one aspect, amino acid substitution is conservative. Substitutions, deletions, insertions or any combination thereof may be combined in a single variant so long as the variant is an immunogenic polypeptide. Variants of pneumolysin typically include any pneumolysin or any fragment of pneumolysin which shares at least 80, 90, 94, 95, 98, or 99% amino acid sequence identity with a wild-type pneumolysin sequence, e.g. SEQ IDs 2-42 from WO2010/071986 (for example SEQ IDs 34, 35, 36, 37, 41). In an embodiment, variants of pneumolysin typically include any pneumolysin or any fragment of pneumolysin which shares at least 80, 90, 94, 95, 98, or 99% amino acid sequence identity with SEQ ID 36 from WO2010/07198. In an embodiment, the present invention includes fragments and/or variants in which several, 5 to 10, 1 to 5, 1 to 3, 1 to 2, 1, up to 10, up to 20, up to 30 or up to 50 amino acids are substituted, deleted, or added in any combination. In another embodiment, the present invention includes fragments and/or variants which comprise a B-cell or T-cell epitope. Such epitopes may be predicted using a combination of 2D-structure prediction, e.g. using the PSIPRED program (from David Jones, Brunel Bioinformatics Group, Dept. Biological Sciences, Brunel University, Uxbridge UB8 3PH, UK) and antigenic index calculated on the basis of the method described by Jameson and Wolf (CABIOS 4:181-186 [1988]). Variants of pneumolysin are described for example in WO04/43376, WO05/108580, WO05/076696, WO10/071,986, WO10/109,325 (SEQ IDs 44, 45 and 46) and WO10/140,119 (SEQ IDs 50 and 51). In an embodiment, the immunogenic composition of the invention comprises a variant of pneumolysin, for example, those described in WO05/108580, WO05/076696, WO10/071,986.

The term “fragment” as used in this specification is a moiety that is capable of eliciting a humoral and/or cellular immune response in a host animal. Fragments of a protein can be produced using techniques known in the art, e.g. recombinantly, by proteolytic digestion, or by chemical synthesis. Internal or terminal fragments of a polypeptide can be generated by removing one or more nucleotides from one end (for a terminal fragment) or both ends (for an internal fragment) of a nucleic acid which encodes the polypeptide. Typically, fragments comprise at least 10, 20, 30, 40 or 50 contiguous amino acids of the full length sequence. Fragments may be readily modified by adding or removing 1, 2, 3, 4, 5, 6, 7, 8, 9, 10, 20, 30, 40 or 50 amino acids from either or both of the N and C termini.

The term “conservative amino acid substitution” as used in this specification involves substitution of a native amino acid residue with a non-native residue such that there is little or no effect on the size, polarity, charge, hydrophobicity, or hydrophilicity of the amino acid residue at that position, and without resulting in decreased immunogenicity. For example, these may be substitutions within the following groups: valine, glycine; glycine, alanine; valine, isoleucine, leucine; aspartic acid, glutamic acid; asparagine, glutamine; serine, threonine; lysine, arginine; and phenylalanine, tyrosine. Conservative amino acid modifications to the sequence of a polypeptide (and the corresponding modifications to the encoding nucleotides) may produce polypeptides having functional and chemical characteristics similar to those of a parental polypeptide.

The term “deletion” as used in this specification is the removal of one or more amino acid residues from the protein sequence. Typically, no more than about from 1 to 6 residues (e.g. 1 to 4 residues) are deleted at any one site within the protein molecule.

The term “insertion” as used in this specification is the addition of one or more non-native amino acid residues in the protein sequence. Typically, no more than about from 1 to 6 residues (e.g. 1 to 4 residues) are inserted at any one site within the protein molecule.

In an embodiment of the invention, pneumolysin and its fragments and/or variants thereof, have an amino acid sequence sharing at least 80, 85, 90, 95, 98, 99 or 100% identity with the wild type sequence for pneumolysin, e.g. SEQ IDs 34, 35, 36, 37, 41 from WO2010/071986. In another embodiment of the invention, pneumolysin and its fragments and/or variants thereof, comprise at least about 15, at least about 20, at least about 40, or at least about 60 contiguous amino acid residues of the wild type sequence for pneumolysin.

Pneumolysin is administered after being detoxified (i.e. rendered non-toxic to a human when provided at a dosage suitable for protection). As used herein, it is understood that the term “dPly” refers to detoxified pneumolysin suitable for medical use (i.e. non toxic). Pneumolysin may be detoxified chemically and/or genetically.

Detoxification of pneumolysin can be conducted by chemical means, e.g. using a crosslinking agent, such as formaldehyde, glutaraldehyde and a cross-linking reagent containing an N-hydroxysuccinomido ester and/or a maleimide group (e.g. GMBS) or a combination of these. In one embodiment the pneumolysin is detoxified by exposure to formaldehyde. Such methods are well known in the art for various toxins; see for example EP1601689B1, WO04/081515, WO2006/032499. The pneumolysin used in chemical detoxification may be a native or recombinant protein or a protein that has been genetically engineered to reduce its toxicity (see below). Fusion proteins of pneumolysin or fragments and/or variants of pneumolysin may also be detoxified by chemical means. Therefore, in an embodiment, immunogenic compositions of the invention may comprise pneumolysin which has been chemically detoxified, e.g. by a formaldehyde treatment.

Pneumolysin can also be genetically detoxified. Thus, the invention encompasses pneumococcal proteins which may be, for example, mutated proteins. The term “mutated” is used herein to mean a molecule which has undergone deletion, addition or substitution of one or more amino acids (e.g. 1, 2, 3, 4, 5, 6, 7, 8, 9, 10, 11 or 12 amino acids), for example by using well known techniques for site directed mutagenesis or any other conventional method. In one embodiment, the molecule has undergone deletion or substitution of 1-15, suitably 10-15 amino acids. The mutated sequences may remove undesirable activities such as membrane permeation, cell lysis, and cytolytic activity against human erythrocytes and other cells, in order to reduce the toxicity, whilst retaining the ability to induce anti-pneumolysin protective and/or neutralizing antibodies following administration to a human. Fusion proteins of pneumolysin or fragments and/or variants of pneumolysin may also be detoxified by genetic means. Any of these modifications may be introduced using standard molecular biology and biochemical techniques. For example, as described above, a mutant pneumolysin protein may be altered so that it is biologically inactive whilst still maintaining its immunogenic epitopes, see, for example, WO90/06951, Berry et al. (Infect Immun, 67:981-985 (1999)) and WO99/03884. For example, a pneumolysin protein may be detoxified by three amino acid substitutions comprising T₆₅ to C, G₂₉₃ to C and C₂₄₈ to A. Another example of a genetically detoxified pneumolysin that can be used in the present invention is SEQ ID 9 from WO2011/075823. Thus, in a further embodiment, immunogenic compositions of the invention may comprise pneumolysin which has been genetically detoxified.

A combination of techniques may be used to detoxify pneumolysin. For example, immunogenic compositions of the invention may comprise pneumolysin which has been chemically and genetically detoxified.

In an embodiment the immunogenic composition comprises 26 μg-45 μg (for example 26 μg-40 μg, 27 μg-39 μg, 27 μg-37 μg, 28 μg-35 μg, 29 μg-33 μg or around 30 μg) of pneumolysin, per human dose.

### PhtD (Poly Histidine Triad D)

In a further embodiment the at least one Streptococcus pneumoniae protein comprises PhtD (Poly Histidine Triad protein D). Note that the terms ‘Pneumococcal Histidine Triad’ and ‘Poly Histidine Triad’ have both been used to describe the PhtX family which includes PhtD. For this reason the terms ‘Pneumococcal Histidine Triad’ and ‘Poly Histidine Triad’ can be considered to be equivalent to one another and therefore interchangeable for the purposes of the present invention.

The term “PhtD” as used herein includes the full length protein with the signal sequence attached or the mature full length protein with the signal peptide (for example 20 amino acids at N-terminus) removed, and fragments, variants and/or fusion proteins thereof, e.g. SEQ ID NO: 4 of WO00/37105. PhtD is also referred to “Sp036D”. In one aspect, PhtD is the full length protein with the signal sequence attached e.g. SEQ ID NO: 4 of WO00/37105. In another aspect, PhtD is a sequence comprising the mature full length protein with the signal peptide (for example 20 amino acids at N-terminus) removed, e.g. amino acids 21-838 of SEQ ID NO: 4 of WO00/37105 (or SEQ ID NO:1 of the present application). Suitably, the PhtD sequence comprises an N-terminal methionine. The present invention also includes PhtD polypeptides which are immunogenic fragments of PhtD, variants of PhtD and/or fusion proteins of PhtD. For example, as described in WO00/37105, WO00/39299, U.S. Pat. No. 6,699,703 and WO09/12588.

Where fragments of PhtD proteins are used (separately or as part of a fusion protein), these fragments will be at least about 15, at least about 20, at least about 40, or at least about 60 contiguous amino acid residues in length, e.g from a PhtD amino acid sequence in WO00/37105 or WO00/39299, such as SEQ ID NO: 4 of WO00/37105. In an embodiment of the invention, immunogenic fragments of PhtD protein comprise at least about 15, at least about 20, at least about 40, or at least about 60 contiguous amino acid residues of the sequence shown in SEQ ID NO: 4 of WO00/37105, wherein said polypeptide is capable of eliciting an immune response specific for said amino acid sequence. In an embodiment, the immunogenic composition of the invention comprises a fragment of PhtD, for example described in WO09/12601, WO01/98334 and WO09/12588. Where fragments of PhtD proteins are used (separately or as part of a fusion protein), each fragment optionally contains one or more histidine triad motif(s) of such polypeptides. A histidine triad motif is the portion of polypeptide that has the sequence HxxHxH (SEQ ID NO:2) where H is histidine and x is an amino acid other than histidine. In an embodiment of the present invention, the or each fragment contains exactly or at least 2, 3, 4 or 5 histidine triad motifs (optionally, with native PhtD sequence between the 2 or more triads, or intra-triad sequence) where the fragment is more than 50, 60, 70, 80, 85, 90, 95, 98, 99 or 100% identical to a native pneumococcal intra-triad PhtD sequence (e.g. the intra-triad sequence shown in SEQ ID NO: 4 of WO00/37105). Fragments of PhtD proteins optionally contain one or more coiled coil regions of such polypeptides. A coiled coil region is a region predicted by “Coils” algorithm Lupus, A et al (1991) Science 252; 1162-1164. In an embodiment of the present invention, the or each fragment contains exactly or at least 2, 3 or 4 coiled coil regions. In an embodiment of the present invention, the or each fragment contains exactly or at least 2, 3 or 4 coiled coil regions where the fragment is more than 50, 60, 70, 80, 90, 95, 96, 98, 99 or 100% identical to a native pneumococcal PhtD sequence (e.g. the sequence shown in SEQ ID NO: 4 of WO00/37105). In another embodiment of the present invention, the or each fragment includes one or more histidine triad motif as well as at least 1, 2, 3 or 4 coiled coil regions. In the case where the PhtD polypeptide is a variant, the variation is generally in a portion thereof other than the histidine triad residues and the coiled-coil region, although variations in one or more of these regions may be made. In accordance with the present invention, a polypeptide variant includes sequences in which one or more amino acids are substituted and/or deleted and/or inserted compared to the wild type sequence. Amino acid substitution may be conservative or non-conservative. In one aspect, amino acid substitution is conservative. Substitutions, deletions, insertions or any combination thereof may be combined in a single variant so long as the variant is an immunogenic polypeptide. Variants of PhtD typically include any fragment or variation of PhtD which shares at least 80, 90, 95, 96, 98, or 99% amino acid sequence identity with a wild-type PhtD sequence, e.g. SEQ ID NO: 4 of WO00/37105. In an embodiment, the present invention includes fragments and/or variants in which several, 5 to 10, 1 to 5, 1 to 3, 1 to 2, 1, up to 10, up to 20, up to 30, or up to 50 amino acids are substituted, deleted, or added in any combination. In another embodiment, the present invention includes fragments and/or variants which comprise a B-cell or T-cell epitope. Such epitopes may be predicted using a combination of 2D-structure prediction, e.g. using the PSIPRED program (from David Jones, Brunel Bioinformatics Group, Dept. Biological Sciences, Brunel University, Uxbridge UB8 3PH, UK) and antigenic index calculated on the basis of the method described by Jameson and Wolf (CABIOS 4:181-186 [1988]). Variants can be produced by conventional molecular biology techniques. Variants as used herein may also include naturally occurring PhtD alleles from alternate Streptococcus strains that exhibit polymorphisms at one or more sites within the homologous PhtD gene.

In an embodiment of the invention, PhtD and its fragments, variants and/or fusion proteins thereof comprise an amino acid sequence sharing at least 80, 85, 90, 95, 96, 97, 98, 99 or 100% identity with amino acid sequence 21 to 838 of SEQ ID NO:4 of WO00/37105 (SEQ ID NO:1). In another embodiment of the invention, PhtD and its fragments, variants and/or fusion proteins thereof have an amino acid sequence sharing at least 80, 85, 90, 95, 96, 97, 98, 99 or 100% identity with amino acid sequence 21 to 838 of SEQ ID NO:4 of WO00/37105 (SEQ ID NO:1). Suitably, PhtD and its fragments, variants and/or fusion proteins thereof comprise an amino acid sequence having an N-terminal methionine. In another embodiment of the invention, PhtD and its fragments, variants and/or fusion proteins thereof comprise at least about 15, at least about 20, at least about 40, or at least about 60 or at least about 100, or at least about 200, or at least about 400 or at least about 800 contiguous amino acid residues of the sequence shown in SEQ ID NO: 4 of WO00/37105.

In an embodiment of the invention, PhtD and its fragments, variants and/or fusion proteins thereof comprise an amino acid sequence sharing at least 80, 85, 90, 95, 96, 97, 98, 99 or 100% identity with amino acid sequence SEQ ID NO:73 of WO00/39299. In another embodiment of the invention, PhtD and its fragments, variants and/or fusion proteins thereof have an amino acid sequence sharing at least 80, 85, 90, 95, 96, 97, 98, 99 or 100% identity with amino acid sequence SEQ ID NO:73 of WO00/39299. In another embodiment of the invention, PhtD and its fragments, variants and/or fusion proteins thereof comprise at least about 15, at least about 20, at least about 40, or at least about 60, or at least about 100, or at least about 200, or at least about 400 or at least about 800 contiguous amino acid residues of the sequence shown in SEQ ID NO: 73 of WO00/39299. In another embodiment of the invention, the PhtD sequence is SEQ ID NO. 1 or 5 from WO2011/075823.

The present invention also includes PhtD proteins which differ from naturally occurring S. pneumoniae polypeptides in ways that do not involve the amino acid sequence. Non-sequence modifications include changes in acetylation, methylation, phosphorylation, carboxylation, or glycosylation. Also within the invention are those with modifications which increase peptide stability; such analogs may contain, for example, one or more non-peptide bonds (which replace the peptide bonds) in the peptide sequence. Also within the invention are analogs that include residues other than naturally occurring L-amino acids, e.g. D-amino acids or non-naturally occurring or synthetic amino acids, e.g. β or γ amino acids, and cyclic analogs.

In one embodiment the immunogenic composition comprises 26 μg-45 μg (for example 26 μg-40 μg, 27 μg-39 μg, 27 μg-37 μg, 28 μg-35 μg, 29 μg-33 μg or around 30 μg) of PhtD, per human dose.

### Subject or Human Host

The uses, methods and immunogenic compositions of the invention provide a an enhanced antibody mediated immune response, for these reasons the uses, methods and immunogenic compositions of the invention are likely to be beneficial, in particular, to subjects or human hosts which are immunocompromised, as the enhancement effect may increase the likelihood that immunocompromised subjects or human hosts are able to respond to the vaccine whilst they may not respond to other vaccines. Similarly this may be true of individuals who are poor responders to vaccination with polysaccharide (as the enhancement effect boost the response to the saccharide).

For this reason the subject or human host may be an immunocompromised human. The term ‘immunocompromised’ refers to a human who is incapable of developing or unlikely to develop a robust immune response, usually as a result of disease, malnutrition or immunosuppressive therapy. Those who can be considered to be immunocompromised include, but are not limited to, subjects with AIDS (or HIV positive), subjects with severe combined immune deficiency (SCID), diabetics, subjects who have had transplants and who are taking immunosuppressive drugs, and those that are receiving chemotherapy. Immunocompromised individuals also includes subjects with most forms of cancer (other than skin cancer), sickle cell anemia, cystic fibrosies, those who do not have a spleen, subjects with endstage kidney disease (dialysis), and those who have been taking corticosteroids on a frequent basis by pill or injection within the last year. Subjects with severe liver, lung or heart disease also may be immunocompromised. In one embodiment the immunocompromised human is a human who has been diagnosed with HIV infection, chronic renal failure, nephritic syndrome, a disease associated with treatment with immunosuppressive drugs or radiation therapy, or congenital immunodeficiency. In a further embodiment the disease associated with treatment with immunosuppressive drugs or ration therapy is selected from the group consisting of malignant neoplasms, leukemias, lymphomas, and Hodgkin disease.

In one embodiment the subject or human host is a human that has been characterized as being an immunocompromised human. The term ‘has been characterized’ means that the subject or human host has been determined to be immunocompromised, for example by a diagnosis of an immunocompromised condition by a doctor or other medical practitioner.

In a further embodiment the methods of the invention comprise a step of characterizing the human host or subject as an immunocompromised human.

Alternatively the subject or human host may be a poor responder to immunisation with polysaccharide. In one embodiment the subject or human host is a human that has been characterized as being a poor responder to immunisation with polysaccharide.

Furthermore immune responses tend to be weaker in the elderly than in young humans. For this reason the methods, uses and immunogenic compositions of the present invention may also be particularly beneficial for elderly populations. For this reason, in one embodiment the subject or human host is an elderly human (optionally wherein the elderly human is more than 50, 55, 60, 65, 70, 75 or 80 years old).

In a further embodiment the host or subject is a human adult, an adult human is a human greater than 18 years of age. In a further embodiment the host or subject is an elderly human suitably an elderly person aged 60, 62, 65, 67, or 70 years and over. In a further embodiment the host or subject is a younger adult (i.e. between 18 and 64 years of age), optionally a younger high-risk adult such as a younger adult working in a heath institution or a young adult with a risk factor such as cardiovascular and pulmonary disease, or diabetes. In a further embodiment the host or subject is an infant human such as a child more than 6 months old, especially children 6 to 23 months of age. Optionally the host or subject is a toddler such as a child between 24 months and 3 years of age

### Dosage

The term “human dose” as used herein means a dose which is in a volume suitable for human use. Generally the final dose volume (vaccine composition volume) administered to a human patient may be between 0.25 to 1.5 ml, 0.2 to 1.0 ml, or 0.4 to 0.6 ml. In an embodiment, a human dose is 0.5 ml. In a further embodiment, a human dose is higher than 0.5 ml, for example 0.6, 0.7, 0.8, 0.9 or 1 ml. In a further embodiment, a human dose is between 1 ml and 1.5 ml. In another embodiment, in particular when the immunogenic composition is for the paediatric population, a human dose may be less than 0.5 ml such as between 0.25 and 0.5 ml.

Further Unconjugated or Conjugated S. pneumoniae Proteins

The immunogenic compositions of the invention may comprise at least one further unconjugated or conjugated Streptococcus pneumoniae protein. For the purposes of the present application the terms S. pneumoniae and Streptococcus pneumoniae are considered to be interchangeable and equivalent to one another. In one embodiment the at least one further unconjugated or conjugated Streptococcus pneumoniae protein is selected from the group consisting of Poly Histidine Triad family (PhtX), Choline Binding Protein Family (CbpX), CbpX truncates, LytX (autolysin encoding gene) family, LytX truncates, CbpX truncate-LytX truncate chimeric proteins, PcpA (pneumococcal choline binding protein A), PspA (pneumococcal surface protein A), PsaA (pneumococcal surface adhesion protein A), Sp128 (streptococcus pneumoniae 128), Sp101 (streptococcus pneumoniae 101), Sp130 (streptococcus pneumoniae 130), SP125 (streptococcus pneumoniae 125) and SP133 (streptococcus pneumoniae 133). In a further embodiment the at least one further unconjugated or conjugated Streptococcus pneumoniae protein is selected from the group consisting of Choline Binding Protein Family (CbpX), CbpX truncates, LytX family, LytX truncates, CbpX truncate-LytX truncate chimeric proteins, PcpA, PspA, PsaA, Sp128, Sp101, Sp130, SP125 and SP133. The Pht (Poly Histidine Triad also known as Poly Histidine Triad) family comprises proteins PhtA (Poly Histidine Triad family A), PhtB (Poly Histidine Triad family B), PhtD (Poly Histidine Triad family B), and PhtE (Poly Histidine Triad family E). The family is characterized by a lipidation sequence, two domains separated by a proline-rich region and several histidine triads, possibly involved in metal or nucleoside binding or enzymatic activity, (3-5) coiled-coil regions, a conserved N-terminus and a heterogeneous C terminus. It is present in all strains of pneumococci tested. Homologous proteins have also been found in other Streptococci and Neisseria. In one embodiment of the invention, the Pht protein of the invention is PhtD. It is understood, however, that the terms Pht A, B, D, and E refer to proteins having sequences disclosed in the citations below as well as naturally-occurring (and man-made) variants thereof that have a sequence homology that is at least 90% identical to the referenced proteins. Optionally it is at least 95% identical or at least 97% identical.

With regards to the PhtX proteins, PhtA is disclosed in WO 98/18930, and is also referred to Sp36. As noted above, it is a protein from the Poly Histidine triad family and has the type II signal motif of LXXC (SEQ ID NO:3). PhtD is disclosed in WO 00/37105, and is also referred to Sp036D. As noted above, it also is a protein from the Poly Histidine triad family and has the type II LXXC signal motif. PhtB is disclosed in WO 00/37105, and is also referred to Sp036B. Another member of the PhtB family is the C3-Degrading Polypeptide, as disclosed in WO 00/17370. This protein also is from the Poly Histidine triad family and has the type II LXXC signal motif. For example, an immunologically functional equivalent is the protein Sp42 disclosed in WO 98/18930. A PhtB truncate (approximately 79 kD) is disclosed in WO99/15675 which is also considered a member of the PhtX family. PhtE is disclosed in WO00/30299 and is referred to as BVH-3. Where any Pht protein is referred to herein, it is meant that immunogenic fragments or fusions thereof of the Pht protein can be used. For example, a reference to PhtX includes immunogenic fragments or fusions thereof from any Pht protein. A reference to PhtD or PhtB is also a reference to PhtDE or PhtBE fusions as found, for example, in WO0198334.

Concerning the Choline Binding Protein family (CbpX), members of that family were originally identified as pneumococcal proteins that could be purified by choline-affinity chromatography. All of the choline-binding proteins are non-covalently bound to phosphorylcholine moieties of cell wall teichoic acid and membrane-associated lipoteichoic acid. Structurally, they have several regions in common over the entire family, although the exact nature of the proteins (amino acid sequence, length, etc.) can vary. In general, choline binding proteins comprise an N terminal region (N), conserved repeat regions (R1 and/or R2), a proline rich region (P) and a conserved choline binding region (C), made up of multiple repeats, that comprises approximately one half of the protein. As used in this application, the term “Choline Binding Protein family (CbpX)” is selected from the group consisting of Choline Binding Proteins as identified in WO97/41151, PbcA, SpsA, PspC, CbpA, CbpD, and CbpG. CbpA is disclosed in WO97/41151. CbpD and CbpG are disclosed in WO00/29434. PspC is disclosed in WO97/09994. PbcA is disclosed in WO98/21337.5 psA is a Choline binding protein disclosed in WO 98/39450. Optionally the Choline Binding Proteins are selected from the group consisting of CbpA, PbcA, SpsA and PspC.

An embodiment of the invention comprises CbpX truncates wherein “CbpX” is defined above and “truncates” refers to CbpX proteins lacking 50% or more of the Choline binding region (C). Optionally such proteins lack the entire choline binding region. Optionally, the such protein truncates lack (i) the choline binding region and (ii) a portion of the N-terminal half of the protein as well, yet retain at least one repeat region (R1 or R2). Optionally, the truncate has 2 repeat regions (R1 and R2). Examples of such embodiments are NR1×R2 and R1×R2 as illustrated in WO99/51266 or WO99/51188, however, other choline binding proteins lacking a similar choline binding region are also contemplated within the scope of this invention.

The LytX family is membrane associated proteins associated with cell lysis. The N-terminal domain comprises choline binding domain(s), however the LytX family does not have all the features found in the CbpA family noted above and thus for the present invention, the LytX family is considered distinct from the CbpX family. In contrast with the CbpX family, the C-terminal domain contains the catalytic domain of the LytX protein family. The family comprises LytA, B and C. With regards to the LytX family, LytA is disclosed in Ronda et al., Eur J Biochem, 164:621-624 (1987). LytB is disclosed in WO 98/18930, and is also referred to as Sp46. LytC is also disclosed in WO 98/18930, and is also referred to as Sp91. An embodiment of the invention comprises LytC.

Another embodiment comprises LytX truncates wherein “LytX” is defined above and “truncates” refers to LytX proteins lacking 50% or more of the Choline binding region. Optionally such proteins lack the entire choline binding region. Yet another embodiment of this invention comprises CbpX truncate-LytX truncate chimeric proteins (or fusions). Optionally this comprises NR1×R2 (or R1×R2) of CbpX and the C-terminal portion (Cterm, i.e., lacking the choline binding domains) of LytX (e.g., LytCCterm or Sp91 Cterm). Optionally CbpX is selected from the group consisting of CbpA, PbcA, SpsA and PspC. Optionally, it is CbpA. Optionally, LytX is LytC (also referred to as Sp91). Another embodiment of the present invention is a PspA or PsaA truncate lacking the choline binding domain (C) and expressed as a fusion protein with LytX. Optionally, LytX is LytC.

With regards to PsaA and PspA, both are know in the art. For example, PsaA and transmembrane deletion variants thereof have been described by Berry & Paton, Infect Immun 1996 December; 64(12):5255-62. PspA and transmembrane deletion variants thereof have been disclosed in, for example, U.S. Pat. No. 5,804,193, WO 92/14488, and WO 99/53940.

With regards to PcpA this protein is known in the art, for example PcpA has been described in WO2011/075823. The term ‘PcpA’ refers to a protein comprising at least 80%, 85%, 90%, 95%, 98%, 99% or 100% identical to SEQ ID NO:2 or 7 from WO2011/075823 or fragments of at least 100, 150, 200, 25 or more consecutive amino acids of SEQ ID NO:2 or 7 from WO2011/075823.

Sp128 and Sp130 are disclosed in WO00/76540. Sp125 is an example of a pneumococcal surface protein with the Cell Wall Anchored motif of LPXTG (SEQ ID NO:4) (where X is any amino acid). Any protein within this class of pneumococcal surface protein with this motif has been found to be useful within the context of this invention, and is therefore considered a further protein of the invention. Sp125 itself is disclosed in WO 98/18930, and is also known as ZmpB—a zinc metalloproteinase. Sp101 is disclosed in WO 98/06734 (where it has the reference # y85993). It is characterized by a Type I signal sequence. Sp133 is disclosed in WO 98/06734 (where it has the reference # y85992). It is also characterized by a Type I signal sequence.

Any of these further S. pneumoniae proteins may be present is an unconjugated or conjugated form. One or more S. pneumoniae proteins is optionally conjugated to an S. pneumoniae saccharide (described in the section entitled S. pneumoniae saccharide conjugates below). Optionally one or more S. pneumoniae proteins is conjugated to a saccharide from a different bacterium.

The term ‘conjugated to’ in this context means that the protein is covalently bonded to a saccharide, in this situation the protein is acting as a carrier protein.

In an embodiment the at least one further unconjugated or conjugated S. pneumoniae protein comprises a Poly Histidine family (PhtX) protein selected from the group consisting of PhtB, PhtE, PhtA, or a PhtBD or PhtDE fusion protein.

In one embodiment the composition comprises 1 μg-100 μg, 1 μg-75 μg, 1 μg-50 μg, 1 μg-25 μg, 1 μg-20 μg, 5 μg-15 μg, 25 μg-40 μg, 28 μg-35 μg, around 10 μg or around 30 μg of the at least one further unconjugated or conjugated S. pneumoniae protein, per human dose, per protein.

Streptococcus pneumoniae Capsular Saccharide Conjugates

In one embodiment the Streptococcus pneumoniae capsular saccharide is a capsular saccharide from a serotype selected from the group consisting of 1, 2, 3, 4, 5, 6A, 6B, 7F, 8, 9N, 9V, 10A, 11A, 12F, 15, 16B, 17F, 18C, 19A, 19F, 20, 22F, 23F and 33F. In a further embodiment the Streptococcus pneumoniae capsular saccharide is a capsular saccharide from a serotype selected from the group consisting of 4, 6B, 7F, 9V, 18C, 19F, and 23F. In addition the immunogenic composition of the invention may further comprise 1 or more (e.g. 7, 8, 9, 10, 11, 12, 13, 14, 15, 16, 17, 18, 19, 20, 21, 22, or 23) further Streptococcus pneumoniae capsular saccharide conjugates. The 1 or more further Streptococcus pneumoniae capsular saccharide conjugates may be selected from the group consisting of 1, 2, 3, 4, 5, 6A, 6B, 7F, 8, 9N, 9V, 10A, 11A, 12F, 15, 16B, 17F, 18C, 19A, 19F, 20, 22F, 23F and 33F. The term capsular saccharide includes capsular polysaccharides and oligosaccharides derivable from the capsular polysaccharide. An oligosaccharide contains at least 4 sugar residues. The terms conjugate and conjugated relate to a capsular saccharide covalently bonded to a carrier protein.

Optionally the total number of saccharide serotypes is less than or equal to 23, optionally the immunogenic composition comprising 10-23 serotypes, 10-16, serotypes 10-15 serotypes, 10-14 serotypes, 10-13 serotypes or 10-12 serotypes. Optionally the immunogenic composition comprises less than 23, 22, 21, 20, 19, 18, 17, 16, 15, 14 or 13 Streptococcus pneumoniae capsular saccharide conjugates. In a further embodiment the immunogenic composition further comprises unconjugated Streptococcus pneumoniae saccharides such that the number of conjugated and unconjugated saccharide serotypes is less than or equal to 23, 22, 21, 20, 19, 18, 17, 16, 15, 14, 13, 12, or 11.

In one embodiment the multivalent immunogenic composition of the invention comprises saccharides from the following serotypes 1, 2, 3, 4, 5, 6A, 6B, 7F, 8, 9N, 9V, 10A, 11A, 12F, 14, 15B, 17F, 18C, 19A, 19F, 20, 22F, 23F and 33F, although it is appreciated that one or two other serotypes could be substituted depending on the age of the recipient receiving the immunogenic composition and the geographical location where the immunogenic composition will be administered. For example, a 7 valent immunogenic composition may comprise polysaccharides from serotypes 4, 6B, 9V, 14, 18C, 19F and 23F. A 10 valent immunogenic composition may comprise the same 7 serotypes and further comprise saccharides derived from serotypes 1, 5 and 7F. A 12 valent immunogenic composition may comprise the same 10 serotypes and further comprises saccharides derived from serotypes 6A and 19A. A 13 valent immunogenic composition may comprise the same 12 serotypes and further comprise a serotype 3 saccharide. A 15 valent immunogenic composition may comprise the same 13 serotypes and further comprise saccharides derived from serotypes 22F and 33F.

The composition in one embodiment includes capsular saccharides derived from serotypes 1, 4, 5, 6B, 7F, 9V, 14, 18C, 19F and 23F (optionally conjugated). In a further embodiment of the invention at least 11 saccharide antigens (optionally conjugated) are included, for example capsular saccharides derived from serotypes 1, 3, 4, 5, 6B, 7F, 9V, 14, 18C, 19F and 23F. In a further embodiment of the invention, at least 12 or 13 saccharide antigens are included, for example a vaccine may comprise capsular saccharides derived from serotypes 1, 4, 5, 6A, 6B, 7F, 9V, 14, 18C, 19A, 19F and 23F or capsular saccharides derived from serotypes 1, 4, 5, 6B, 7F, 9V, 14, 18C, 19A, 19F, 22F and 23F, although further saccharide antigens, for example 23 valent (such as serotypes 1, 2, 3, 4, 5, 6B, 7F, 8, 9N, 9V, 10A, 11A, 12F, 14, 15B, 17F, 18C, 19A, 19F, 20, 22F, 23F and 33F), are also contemplated by the invention.

In general the antibody-mediated opsonic activity against the serotypes from which the at least 1 further saccharides are derived is also enhanced in the method, immunogenic composition or use of the present invention, in a further embodiment the opsonophagocytic response to 2, 3, 4, 5, 6, 7, 8, 9, 10, 11, 12, 13, 14, 15, 16, 17, 18, 19, 20, 21, 22 or 23 of the further Streptococcus pneumoniae capsular saccharides is enhanced.

In one embodiment the 1 or more further Streptococcus pneumoniae saccharide conjugates comprises a conjugated serotype 1 saccharide (optionally wherein the immunogenic composition enhances antibody-mediated opsonic activity against serotype 1 Streptococcus pneumoniae). In a further embodiment the 1 or more further Streptococcus pneumoniae saccharide conjugates comprises a conjugated serotype 4 saccharide (optionally wherein immunogenic composition enhances antibody-mediated opsonic activity against serotype 4 Streptococcus pneumoniae). In a further embodiment the 1 or more further Streptococcus pneumoniae saccharide conjugates comprises a conjugated serotype 5 saccharide (optionally wherein the immunogenic composition enhances antibody-mediated opsonic activity against serotype 5 Streptococcus pneumoniae). In a further embodiment the 1 or more further Streptococcus pneumoniae saccharide conjugates comprises a conjugated serotype 6B saccharide (optionally wherein the immunogenic composition enhances antibody-mediated opsonic activity against serotype 6B Streptococcus pneumoniae). In a further embodiment the 1 or more further Streptococcus pneumoniae saccharide conjugates comprises a conjugated serotype 7F saccharide (optionally wherein the immunogenic composition enhances antibody-mediated opsonic activity against serotype 7F Streptococcus pneumoniae). In a further embodiment the 1 or more further Streptococcus pneumoniae saccharide conjugates comprises a conjugated serotype 9V saccharide (optionally wherein the immunogenic composition enhances antibody-mediated opsonic activity against serotype 9V Streptococcus pneumoniae). In a further embodiment the 1 or more further Streptococcus pneumoniae saccharide conjugates comprises a conjugated serotype 14 saccharide (optionally wherein the immunogenic composition enhances antibody-mediated opsonic activity against serotype 14 Streptococcus pneumoniae). In a further embodiment the 1 or more further Streptococcus pneumoniae saccharide conjugates comprises a conjugated serotype 18C saccharide (optionally wherein the immunogenic composition enhances antibody-mediated opsonic activity against serotype 18C Streptococcus pneumoniae). In a further embodiment the 1 or more further Streptococcus pneumoniae saccharide conjugates comprises a conjugated serotype 19F saccharide (optionally wherein the immunogenic composition enhances antibody-mediated opsonic activity against serotype 19F Streptococcus pneumoniae). In a further embodiment the 1 or more further Streptococcus pneumoniae saccharide conjugates comprises a conjugated serotype 23F saccharide (optionally wherein immunogenic composition enhances antibody-mediated opsonic activity against serotype 23F Streptococcus pneumoniae). In a further embodiment the 1 or more further Streptococcus pneumoniae saccharide conjugates comprises a conjugated serotype 3 saccharide (optionally wherein the immunogenic composition enhances antibody-mediated opsonic activity against serotype 3 Streptococcus pneumoniae). In one embodiment the 1 or more further Streptococcus pneumoniae saccharide conjugates comprises a conjugated serotype 6A saccharide (optionally wherein the immunogenic composition enhances antibody-mediated opsonic activity against serotype 6A Streptococcus pneumoniae). In a further embodiment the 1 or more further Streptococcus pneumoniae saccharide conjugates comprises a conjugated serotype 19A saccharide (optionally wherein the immunogenic composition enhances antibody-mediated opsonic activity against serotype 19A Streptococcus pneumoniae). In a further embodiment the 1 or more further Streptococcus pneumoniae saccharide conjugates comprises a conjugated serotype 33F saccharide (optionally wherein the immunogenic composition enhances antibody-mediated opsonic activity against serotype 33F Streptococcus pneumoniae).

The term “carrier protein” is intended to cover both small peptides and large polypeptides (>10 kDa). The carrier protein may be any peptide or protein. It may comprise one or more T-helper epitopes. The carrier protein may be tetanus toxoid (TT), tetanus toxoid fragment C, non-toxic mutants of tetanus toxin [note all such variants of TT are considered to be the same type of carrier protein for the purposes of this invention], polypeptides comprising tetanus toxin T-cell epitopes such as N19 (WO2006/067632), diphtheria toxoid (DT), CRM197 (cross reacting material 197), other non-toxic mutants of diphtheria toxin [such as CRM176, CRM 197, CRM228, CRM 45 (Uchida et al J. Biol. Chem. 218; 3838-3844, 1973); CRM 9, CRM 45, CRM102, CRM 103 and CRM107 (where CRM stands for cross reacting material) and other mutations described by Nicholls and Youle in Genetically Engineered Toxins, Ed: Frankel, Maecel Dekker Inc, 1992; deletion or mutation of Glu-148 to Asp, Gln or Ser and/or Ala 158 to Gly and other mutations disclosed in U.S. Pat. No. 4,709,017 or U.S. Pat. No. 4,950,740; mutation of at least one or more residues Lys 516, Lys 526, Phe 530 and/or Lys 534 and other mutations disclosed in U.S. Pat. No. 5,917,017 or U.S. Pat. No. 6,455,673; or fragment disclosed in U.S. Pat. No. 5,843,711] (note all such variants of DT are considered to be the same type of carrier protein for the purposes of this invention), pneumococcal pneumolysin (Kuo et al (1995) Infect Immun 63; 2706-13), OMPC (outer membrane protein C) (meningococcal outer membrane protein—usually extracted from N. meningitidis serogroup B—EP0372501), synthetic peptides (EP0378881, EP0427347), heat shock proteins (WO 93/17712, WO 94/03208), pertussis proteins (WO 98/58668, EP0471177), cytokines, lymphokines, growth factors or hormones (WO 91/01146), artificial proteins comprising multiple human CD4+ T cell epitopes from various pathogen derived antigens (Falugi et al (2001) Eur J Immunol 31; 3816-3824) such as N19 protein (Baraldoi et al (2004) Infect Immun 72; 4884-7) pneumococcal surface protein PspA (pneumococcal surface protein A) (WO 02/091998), iron uptake proteins (WO 01/72337), toxin A or B of C. difficile (WO 00/61761), H. influenzae Protein D (EP594610 and WO 00/56360), pneumococcal PhtA (Poly Histidine triad protein A) (WO 98/18930, also referred to Sp36), pneumococcal PhtD (Poly Histidine triad protein D (disclosed in WO 00/37105, and is also referred to Sp036D), pneumococcal PhtB (Poly Histidine triad protein B) (disclosed in WO 00/37105, and is also referred to Sp036B), or PhtE (Poly Histidine triad protein E) (disclosed in WO00/30299 and is referred to as BVH-3).

In one embodiment the Streptococcus pneumoniae capsular saccharide conjugate is conjugated to a carrier protein independently selected from the group consisting of tetanus toxoid (TT), fragment C of TT, diphtheria toxoid, CRM197 (cross reacting material 197), detoxified pneumolysin, protein D (from H. influenzae), PhtD, PhtDE and N19. In a further embodiment the 1 or more Streptococcus pneumoniae capsular saccharide conjugates are all independently conjugated to CRM197.

In one embodiment the 1 or more further Streptococcus pneumoniae saccharide conjugates are independently conjugated to a carrier protein selected from the group consisting of tetanus toxoid (TT), fragment C of TT, diphtheria toxoid, CRM197, Pneumolysin, protein D, PhtD, PhtDE and N19. In a further embodiment the 1 or more further Streptococcus pneumoniae saccharide conjugates are all independently conjugated to CRM197.

In one embodiment the immunogenic composition comprises a saccharide from serotype 1 conjugated to protein D or CRM197. In a further embodiment the immunogenic composition comprises a saccharide from serotype 4 conjugated to protein D or CRM197. In a further embodiment the immunogenic composition comprises a saccharide from serotype 5 conjugated to protein D or CRM197. In a further embodiment the immunogenic composition comprises a saccharide from serotype 6B conjugated to protein D or CRM197. In a further embodiment the composition comprises a saccharide from serotype 6B conjugated to protein D or CRM197. In a further embodiment the immunogenic composition comprises a saccharide from serotype 7F conjugated to protein D or CRM197. In a further embodiment the immunogenic composition comprises a saccharide from serotype 9V conjugated to protein D or CRM197. In a further embodiment the immunogenic composition comprises a saccharide from serotype 14 conjugated to protein D or CRM197. In a further embodiment the immunogenic composition comprises a saccharide from serotype 18C conjugated to Tetanus Toxoid or CRM197. In a further embodiment the immunogenic composition comprises a saccharide from serotype 19F conjugated to Diphtheria Toxoid or CRM197. In a further embodiment the immunogenic composition comprises a saccharide from serotype 23F conjugated to protein D or CRM197. In a further embodiment the immunogenic composition comprises a saccharide from serotype 3 conjugated to protein D or CRM197. In a further embodiment the immunogenic composition comprises a saccharide from serotype 6A conjugated to protein D or CRM197. In a further embodiment the immunogenic composition comprises a saccharide from serotype 19A conjugated to protein D or CRM197.

In one embodiment a minority of the 1 or more further Streptocoocus pneumoniae saccharide conjugates are conjugated to protein D. In general the term ‘minority’ refers to less than half. For example, in a 10 valent S. pneumoniae vaccine, 2, 3, 4 or 5 of the capsular saccharides from different serotypes are conjugated to protein D. For example, in a 11 valent S. pneumoniae vaccine, 2, 3, 4 or 5 of the capsular saccharides from different serotypes are conjugated to protein D. For example, in a 12 valent S. pneumoniae vaccine, 2, 3, 4, 5 or 6 of the capsular saccharides from different serotypes are conjugated to protein D. For example, in a 13 valent S. pneumoniae vaccine, 2, 3, 4, 5 or 6 of the capsular saccharides from different serotypes are conjugated to protein D. For example, in a 14 valent S. pneumoniae vaccine, 2, 3, 4, 5, 6 or 7 of the capsular saccharides from different serotypes are conjugated to protein D. For example, in a 15 valent S. pneumoniae vaccine, 2, 3, 4, 5, 6 or 7 of the capsular saccharides from different serotypes are conjugated to protein D. For example, in a 16 valent S. pneumoniae vaccine, 2, 3, 4, 5, 6, 7 or 8 of the capsular saccharides from different serotypes are conjugated to protein D. For example, in a 17 valent S. pneumoniae vaccine, 2, 3, 4, 5, 6, 7 or 8 of the capsular saccharides from different serotypes are conjugated to protein D. For example, in a 18 valent S. pneumoniae vaccine, 2, 3, 4, 5, 6, 7, 8 or 9 of the capsular saccharides from different serotypes are conjugated to protein D. For example, in a 19 valent S. pneumoniae vaccine, 2, 3, 4, 5, 6, 7, 8 or 9 of the capsular saccharides from different serotypes are conjugated to protein

D. In a further embodiment the immunogenic composition comprises between 1-20, 1-18, 1-16, −1-14, 1-12, 1-10, 1-9, 1-8, 1-7, 1-6, 1-5, 1-4, or 1-2 Streptococcus pneumoniae capsular saccharide conjugated to protein D. In one embodiment the immunogenic composition comprises at least one Streptococcus pneumoniae capsular saccharides conjugated to diphtheria toxoid. In a further embodiment the immunogenic composition comprises 19F conjugated to diphtheria toxoid. In one embodiment the immunogenic composition comprises at least one Streptococcus pneumoniae capsular saccharides conjugated to tetanus toxoid. In a further embodiment the immunogenic composition comprises 18C conjugated to tetanus toxoid.

In one embodiment the immunogenic composition comprises Streptococcus pneumoniae conjugates comprising a serotype 1 saccharide conjugated to protein D, a serotype 4 saccharide conjugated to protein D, a serotype 5 saccharide conjugated to protein D, a serotype 6B saccharide conjugated to protein D, a serotype 7F saccharide conjugated to protein D, a serotype 9V saccharide conjugated to protein D, a serotype 14 saccharide conjugated to protein D, a serotype 23F saccharide conjugated to protein D, a serotype 18C saccharide conjugated to tetanus toxoid and a 19F saccharide conjugated to diphtheria toxoid.

Optionally the ratio of carrier protein to S. pneumoniae saccharide is between 1:5 and 5:1; 1:2 and 2.5:1; 1:1 and 2:1 (w/w). In an embodiment, the majority of the conjugates, for example 6, 7, 8, 9 or more of the conjugates have a ratio of carrier protein to saccharide that is greater than 1:1, for example 1.1:1, 1.2:1, 1.3:1, 1.4:1, 1.5:1 or 1.6:1.

The term “saccharide” throughout this specification may indicate polysaccharide or oligosaccharide and includes both. Polysaccharides are isolated from bacteria and may be sized to some degree by known methods (see for example EP497524 and EP497525) and optionally by microfluidisation. Polysaccharides can be sized in order to reduce viscosity in polysaccharide samples and/or to improve filterability for conjugated products. Oligosaccharides have a low number of repeat units (typically 5-30 repeat units) and are typically hydrolysed polysaccharides.

Capsular polysaccharides of Streptococcus pneumoniae comprise repeating oligosaccharide units which may contain up to 8 sugar residues. For a review of the oligosaccharide units for the key Streptococcus pneumoniae serotypes see JONES, Christopher. Vaccines based on the cell surface carbohydrates of pathogenic bacteria. An. Acad. Bras. Ciênc., June 2005, vol. 77, no. 2, p. 293-324. ISSN 0001-3765. In one embodiment, a capsular saccharide antigen may be a full length polysaccharide, however in others it may be one oligosaccharide unit, or a shorter than native length saccharide chain of repeating oligosaccharide units. In one embodiment, all of the saccharides present in the vaccine are polysaccharides. Full length polysaccharides may be “sized” i.e. their size may be reduced by various methods such as acid hydrolysis treatment, hydrogen peroxide treatment, sizing by Emulsiflex® followed by a hydrogen peroxide treatment to generate oligosaccharide fragments or microfluidization.

### Conjugation

The saccharide conjugates present in the immunogenic compositions of the invention may be conjugated to a carrier protein using any conjugation technique.

In an embodiment, the Streptococcus pneumoniae saccharide is conjugated to the carrier protein via a linker, for instance a bifunctional linker. The linker is optionally heterobifunctional or homobifunctional, having for example a reactive amino group and a reactive carboxylic acid group, 2 reactive amino groups or two reactive carboxylic acid groups. The linker has for example between 4 and 20, 4 and 12, 5 and 10 carbon atoms. A possible linker is ADH. Other linkers include B-propionamido (WO 00/10599), nitrophenyl-ethylamine (Gever et al (1979) Med. Microbiol. Immunol. 165; 171-288), haloalkyl halides (U.S. Pat. No. 4,057,685), glycosidic linkages (U.S. Pat. No. 4,673,574, U.S. Pat. No. 4,808,700), hexane diamine and 6-aminocaproic acid (U.S. Pat. No. 4,459,286). In an embodiment, ADH is used as a linker for conjugating saccharide from serotype 18C.

The saccharide conjugates present in the immunogenic compositions of the invention may be prepared by any known coupling technique. The conjugation method may rely on activation of the saccharide with 1-cyano-4-dimethylamino pyridinium tetrafluoroborate (CDAP) to form a cyanate ester. The activated saccharide may thus be coupled directly or via a spacer (linker) group to an amino group on the carrier protein. For example, the spacer could be cystamine or cysteamine to give a thiolated polysaccharide which could be coupled to the carrier via a thioether linkage obtained after reaction with a maleimide-activated carrier protein (for example using GMBS) or a haloacetylated carrier protein (for example using iodoacetimide [e.g. ethyl iodoacetimide HCl] or N-succinimidyl bromoacetate or SIAB, or SIA, or SBAP). Optionally, the cyanate ester (optionally made by CDAP chemistry) is coupled with hexane diamine or ADH and the amino-derivatised saccharide is conjugated to the carrier protein using carbodiimide (e.g. EDAC or EDC) chemistry via a carboxyl group on the protein carrier. Such conjugates are described in PCT published application WO 93/15760 Uniformed Services University and WO 95/08348 and WO 96/29094.

Other suitable techniques use carbodiimides, carbiinides, hydrazides, active esters, norborane, p-nitrobenzoic acid, N-hydroxysuccinimide, S—NHS, EDC, TSTU. Many are described in WO 98/42721. Conjugation may involve a carbonyl linker which may be formed by reaction of a free hydroxyl group of the saccharide with CDI (Bethell et al J. Biol. Chem. 1979, 254; 2572-4, Hearn et al J. Chromatogr. 1981. 218; 509-18) followed by reaction of with a protein to form a carbamate linkage. This may involve reduction of the anomeric terminus to a primary hydroxyl group, optional protection/deprotection of the primary hydroxyl group' reaction of the primary hydroxyl group with CDI to form a CDI carbamate intermediate and coupling the CDI carbamate intermediate with an amino group on a protein.

The conjugates can also be prepared by direct reductive amination methods as described in U.S. Pat. No. 4,365,170 (Jennings) and U.S. Pat. No. 4,673,574 (Anderson). Other methods are described in EP-0-161-188, EP-208375 and EP-0-477508.

A further method involves the coupling of a cyanogen bromide (or CDAP) activated saccharide derivatised with adipic acid dihydrazide (ADH) to the protein carrier by Carbodiimide condensation (Chu C. et al Infect. Immunity, 1983 245 256), for example using EDAC (1-ethyl-2-(3-dimethylaminopropyl)carbodiimide).

In an embodiment, a hydroxyl group (optionally an activated hydroxyl group for example a hydroxyl group activated to make a cyanate ester [e.g. using CDAP]) on a saccharide is linked to an amino or carboxylic group on a protein either directly or indirectly (through a linker). Where a linker is present, a hydroxyl group on a saccharide is optionally linked to an amino group on a linker, for example by using CDAP conjugation. A further amino group in the linker for example ADH) may be conjugated to a carboxylic acid group on a protein, for example by using carbodiimide chemistry, for example by using EDAC. In an embodiment, the pneumococcal capsular saccharide(s) is conjugated to the linker first before the linker is conjugated to the carrier protein. Alternatively the linker may be conjugated to the carrier before conjugation to the saccharide.

A combination of techniques may also be used, with some saccharide-protein conjugates being prepared by CDAP, and some by reductive amination.

In general the following types of chemical groups on a protein carrier can be used for coupling/conjugation:

A) Carboxyl (for instance via aspartic acid or glutamic acid). In one embodiment this group is linked to amino groups on saccharides directly or to an amino group on a linker with carbodiimide chemistry e.g. with EDAC.
B) Amino group (for instance via lysine). In one embodiment this group is linked to carboxyl groups on saccharides directly or to a carboxyl group on a linker with carbodiimide chemistry e.g. with EDAC. In another embodiment this group is linked to hydroxyl groups activated with CDAP or CNBr on saccharides directly or to such groups on a linker; to saccharides or linkers having an aldehyde group; to saccharides or linkers having a succinimide ester group.
C) Sulphydryl (for instance via cysteine). In one embodiment this group is linked to a bromo or chloro acetylated saccharide or linker with maleimide chemistry. In one embodiment this group is activated/modified with bis diazobenzidine.
D) Hydroxyl group (for instance via tyrosine). In one embodiment this group is activated/modified with bis diazobenzidine.
E) Imidazolyl group (for instance via histidine). In one embodiment this group is activated/modified with bis diazobenzidine.
F) Guanidyl group (for instance via arginine).
G) Indolyl group (for instance via tryptophan).

On a saccharide, in general the following groups can be used for a coupling: OH, COOH or NH2. Aldehyde groups can be generated after different treatments known in the art such as: periodate, acid hydrolysis, hydrogen peroxide, etc.

Direct Coupling Approaches:

Saccharide-OH+CNBr or CDAP---->cyanate ester+NH2-Prot---->conjugate
Saccharide-aldehyde+NH2-Prot---->Schiff base+NaCNBH3---->conjugate
Saccharide-COOH+NH2-Prot+EDAC---->conjugate
Saccharide-NH2+COOH-Prot+EDAC---->conjugate

Indirect coupling via spacer (linker) approaches:

Saccharide-OH+CNBr or CDAP---->cyanate ester+NH2----NH2---->saccharide----NH2+
COOH-Prot+EDAC---->conjugate
Saccharide-OH+CNBr or CDAP---->cyanate ester+NH2-----SH----->saccharide----SH+SH-Prot (native Protein with an exposed cysteine or obtained after modification of amino groups of the protein by SPDP for instance)----->saccharide-S—S-Prot
Saccharide-OH+CNBr or CDAP--->cyanate ester+NH2----SH------->saccharide----SH+maleimide-Prot (modification of amino groups)---->conjugate
Saccharide-OH+CNBr or CDAP--->cyanate ester+NH2-----SH--->Saccharide-SH+haloacetylated-Prot---->Conjugate
Saccharide-COOH+EDAC+NH2-----NH2--->saccharide------NH2+EDAC+COOH-Prot---->conjugate
Saccharide-COOH+EDAC+NH2----SH----->saccharide----SH+SH-Prot (native Protein with an exposed cysteine or obtained after modification of amino groups of the protein by SPDP for instance)----->saccharide-S—S-Prot
Saccharide-COOH+EDAC+NH2----SH----->saccharide----SH+maleimide-Prot (modification of amino groups)---->conjugate
Saccharide-COOH+EDAC+NH2----SH--->Saccharide-SH+haloacetylated-Prot---->Conjugate
Saccharide-Aldehyde+NH2-----NH2---->saccharide---NH2+EDAC+COOH-Prot---->conjugate

Note: instead of EDAC above, any suitable carbodiimide may be used.

In summary, the types of protein carrier chemical group that may be generally used for coupling with a saccharide are amino groups (for instance on lysine residues), COOH groups (for instance on aspartic and glutamic acid residues) and SH groups (if accessible) (for instance on cysteine residues.

The modes of conjugation can be used to conjugate the Streptococcus pneumoniae capsular saccharide and/or the 1 or more further Streptococcus pneumoniae saccharides to a carrier protein. In one embodiment wherein the Streptococcus pneumoniae capsular saccharide is conjugated to a carrier protein through CDAP chemistry. In a further embodiment the 1 or more further Streptococcus pneumoniae saccharide conjugates are conjugated to a carrier protein through reductive amination. In a further embodiment the 1 or more further Streptococcus pneumoniae saccharide conjugates are conjugated to a carrier protein through CDAP chemistry.

In one embodiment at least 1, 2, 3, 4, 5, 6, 7, 8, 9, 10, 11, 12, 13, 14, 15, 16, 17, 18, 19, 20, 21, 22, or 23 S. pneumoniae saccharides are conjugated to a carrier protein through reductive amination. In one embodiment less than 23, 22, 21, 20, 19, 18, 17, 16, 15, 14, 13, 12, 11, 10, 9, 8, 7, 6, 5, 4, 3, 2 or 1 S. pneumoniae saccharides are conjugated to a carrier protein through reductive amination. In one embodiment between 1 and 23, 2 and 22, 3 and 21, 4 and 20, 5 and 19, 6 and 18, 7 and 17, 8 and 16, 9 and 15, 10 and 14, 11 and 13, 1 and 23, and 22, 1 and 21, 1 and 20, 1 and 19, 1 and 18, 1 and 17, 1 and 16, 1 and 15, 1 and 14, 1 and 13, 1 and 12, 1 and 11, 1 and 10, 1 and 9, 1 and 8, 1 and 7, 1 and 6, 1 and 5, 1 and 4, 1 and 3, or 1 and 2 S. pneumoniae saccharide are conjugated to a carrier protein through reductive amination. In a further embodiment all of the S. pneumoniae capsular saccharides are conjugated to a carrier protein through reductive amination.

In one embodiment at least 1, 2, 3, 4, 5, 6, 7, 8, 9, 10, 11, 12, 13, 14, 15, 16, 17, 18, 19, 20, 21, 22, or 23 S. pneumoniae saccharides are conjugated to a carrier protein through CDAP chemistry. In one embodiment less than 23, 22, 21, 20, 19, 18, 17, 16, 15, 14, 13, 12, 11, 10, 9, 8, 7, 6, 5, 4, 3, 2 or 1 S. pneumoniae saccharides are conjugated to a carrier protein through CDAP chemistry. In one embodiment between 1 and 23, 2 and 22, 3 and 21, 4 and 20, 5 and 19, 6 and 18, 7 and 17, 8 and 16, 9 and 15, 10 and 14, 11 and 13, 1 and 23, and 22, 1 and 21, and 23, 10 and 22, 10 and 21, 10 and 20, 10 and 19, 10 and 18, 10 and 17, 10 and 16, 10 and 15, 10 and 14, 10 and 13, 10 and 12, 10 and 11, 1 and 20, 1 and 19, 1 and 18, 1 and 17, 1 and 16, 1 and 15, 1 and 14, 1 and 13, 1 and 12, 1 and 11, 1 and 10, 1 and 9, 1 and 8, 1 and 7, 1 and 6, 1 and 5, 1 and 4, 1 and 3, or 1 and 2 S. pneumoniae saccharide are conjugated to a carrier protein through CDAP chemistry. In a further embodiment all of the S. pneumoniae capsular saccharides are conjugated to a carrier protein through CDAP chemistry.

In one embodiment the immunogenic composition of the invention comprises at least 1, 2, 3, 4, 5, 6, 7, 8, 9, 10, 11, 12, 13, 14, 15, 16, 17, 18, 19, 20, 21 or 22 saccharides conjugated to a carrier protein through reductive amination and comprises at least 1, 2, 3, 4, 5, 6, 7, 8, 9, 10, 11, 12, 13, 14, 15, 16, 17, 18, 19, 20, 21, or 22 saccharides conjugated to a carrier protein through a chemistry other than reductive amination for example CDAP chemistry.

In an embodiment capsular saccharides from at least one of the serotypes selected from the group consisting of serotypes 1, 3, 19A and 19F are conjugated through a chemistry other than reductive amination and at least one of the serotypes selected from the group consisting of serotypes 4, 5, 6A, 6B, 6C, 7F, 9V, 14, 18C and 23F are conjugated through reductive amination. In an embodiment, the immunogenic composition of the invention comprises S. pneumoniae capsular saccharide(s) from serotype 1 or 3 or 19A or 19F; 1 and 3; 1 and 19A; 1 and 19F; 3 and 19A; 3 and 19F; 19A and 19F; 1, 3 and 19A; 1, 3 and 19F, 1, 19A and 19F; 3, 19A and 19F or 1, 3, 19A and 19F conjugated to a protein carrier through a chemistry other than reductive animation. In an embodiment, 19F is conjugated to a carrier protein through a chemistry other than reductive amination. In an embodiment, the immunogenic composition of the invention comprises S. pneumoniae capsular saccharide from serotype 1 or 3 or 19A or 19F; 1 and 3; 1 and 19A; 1 and 19F; 3 and 19A; 3 and 19F; 19A and 19F; 1, 3 and 19A; 1, 3 and 19F, 1, 19A and 19F; 3, 19A and 19F or 1, 3, 19A and 19F conjugated to a protein carrier through cyanylation chemistry such as CDAP chemistry. In an embodiment, 19F is conjugated to a carrier protein by CDAP chemistry. In an embodiment of the invention, the following S. pneumoniae capsular saccharide or group thereof is conjugated to a carrier protein by reductive amination; serotype 4, 5, 6A, 6B, 7F, 9V, 14, 18C or 23F, 4 and 5, 4 and 6A, 4 and 6B, 4 and 7F, 4 and 9V, 4 and 14, 4 and 18C, 4 and 23F, 5 and 6A, 5 and 6B, 5 and 7F, 5 and 9V, 5 and 14, 5 and 18C, 5 and 23F, 6A and 6B, 6A and 7F, 6A and 9V, 6A and 14, 6A and 18C, 6A and 23F, 6B and 7F, 6B and 9V, 6B and 14, 6B and 18C, 6B and 23F, 7F and 9V, 7F and 14, 7F and 18C, 7F and 23F, 9V and 14, 9V and 18C, 9V and 23F, 14 and 18C, 14 and 23F or 18C and 23F. In an embodiment, 23F is conjugated to a carrier protein by reductive amination chemistry.

### Doses of Saccharide Conjugates

In general, the immunogenic composition of the invention may comprise a dose of each saccharide conjugate between 0.1 and 20 μg, 1 and 5 μg, 1 and 10 μg or 1 and 3 μg of saccharide per conjugate.

In an embodiment, the immunogenic composition of the invention contains each S. pneumoniae capsular saccharide at a dose of between 0.1-20 μg; 0.5-10 μg; 0, 5-5 μg or 1-3 μg of saccharide. In an embodiment, capsular saccharides may be present at different dosages, for example some capsular saccharides may be present at a dose of exactly 1 μg or some capsular saccharides may be present at a dose of exactly 3 μg. In an embodiment the immunogenic composition comprises Streptococcus pneumoniae comprises capsular saccharide conjugates of saccharides from serotypes 1, 5, 6B, 7F, 9V, 14 and 23F (and optionally 6A and/or 3) at dosages of 1 μg of saccharide per conjugate. In an embodiment, saccharides from serotypes 3, 18C and 19F (or 4, 18C and 19F) are present at a higher dose than other saccharides. In one aspect of this embodiment, serotypes 3, 18C and 19F (or 4, 18C and 19F) are present at a dose of around or exactly 3 μg whilst other saccharides in the immunogenic composition are present at a dose of around or exactly 1 μg. In one embodiment serotypes 1, 5, 6B, 7F, 9V, 14 and 23F are present at a dose of around or exactly 1 μg.

### Adjuvants

In one embodiment the composition does not comprise an adjuvant comprising QS21, Monophosphoryl lipid A (MPL), phospholipid and sterol, presented in the form of a liposome. Compositions comprising adjuvant comprising QS21, Monophosphoryl lipid A (MPL), phospholiopid and sterol presented in the form of a liposome are described in PCT application number WO2012/156391. In one embodiment the immunogenic composition does not comprise an adjuvant comprising QS 21. In a further embodiment the immunogenic composition does not comprise an adjuvant comprising Monophosphoryl lipid A (MPL). In one embodiment the immunogenic composition does not comprise either QS21 or MPL. QS21 is an adjuvant derived from the tree Quillaja saponaria.

In one embodiment the immunogenic composition further comprises an adjuvant.

Suitable adjuvants include, but are not limited to, aluminium salts (aluminium phosphate or aluminium hydroxide), monophosphoryl lipid A (for example 3D-MPL), saponins (for example QS21), oil in water emulsions, blebs or outer membrane vesicle preparations from Gram negative bacterial strains (such as those taught by WO02/09746), lipid A or derivatives thereof, alkyl glucosamide phosphates or combinations of two or more of these adjuvants. In one embodiment the adjuvant is aluminium phosphate. In a further embodiment the adjuvant comprises 100-750, 150-600, 200-500, 250-450, 300-400, or around 350 μg aluminium as aluminium phosphate per human dose. In a further embodiment the adjuvant is aluminium hydroxide. In one embodiment the adjuvant does not comprise an oil in water adjuvant.

### Vaccines

The present invention provides a vaccine comprising the immunogenic compositions of the invention. Embodiments herein relating to “immunogenic compositions” of the invention are also applicable to embodiments relating to “vaccines” of the invention, and vice versa. In an embodiment, the vaccine comprises the immunogenic composition of the invention and a pharmaceutically acceptable excipient.

The vaccines of the invention may be administered by any suitable delivery route, such as intradermal, mucosal e.g. intranasal, oral, intramuscular or subcutaneous. Other delivery routes are well known in the art. Vaccine preparation is generally described in Vaccine Design (“The subunit and adjuvant approach” (eds Powell M. F. & Newman M. J.) (1995) Plenum Press New York).

In one aspect, the immunogenic composition of the invention is administered by the intramuscular delivery route. Intramuscular administration may be to the thigh or the upper arm. Injection is typically via a needle (e.g. a hypodermic needle), but needle-free injection may alternatively be used. A typical intramuscular dose is 0.5 ml.

Intradermal administration of the vaccine forms an embodiment of the present invention. Human skin comprises an outer “horny” cuticle, called the stratum corneum, which overlays the epidermis. Underneath this epidermis is a layer called the dermis, which in turn overlays the subcutaneous tissue. The conventional technique of intradermal injection, the “mantoux procedure”, comprises steps of cleaning the skin, and then stretching with one hand, and with the bevel of a narrow gauge needle (26 to 31 gauge) facing upwards the needle is inserted at an angle of between 10 to 15°. Once the bevel of the needle is inserted, the barrel of the needle is lowered and further advanced whilst providing a slight pressure to elevate it under the skin. The liquid is then injected very slowly thereby forming a bleb or bump on the skin surface, followed by slow withdrawal of the needle.

More recently, devices that are specifically designed to administer liquid agents into or across the skin have been described, for example the devices described in WO99/34850 and EP1092444, also the jet injection devices described for example in WO01/13977, U.S. Pat. No. 5,480,381, U.S. Pat. No. 5,599,302, U.S. Pat. No. 5,334,144, U.S. Pat. No. 5,993,412, U.S. Pat. No. 5,649,912, U.S. Pat. No. 5,569,189, U.S. Pat. No. 5,704,911, U.S. Pat. No. 5,383,851, U.S. Pat. No. 5,893,397, U.S. Pat. No. 5,466,220, U.S. Pat. No. 5,339,163, U.S. Pat. No. 5,312,335, U.S. Pat. No. 5,503,627, U.S. Pat. No. 5,064,413, U.S. Pat. No. 5,520,639, U.S. Pat. No. 4,596,556, U.S. Pat. No. 4,790,824, U.S. Pat. No. 4,941,880, U.S. Pat. No. 4,940,460, WO97/37705 and WO97/13537. Alternative methods of intradermal administration of the vaccine preparations may include conventional syringes and needles, or devices designed for ballistic delivery of solid vaccines (WO99/27961), or transdermal patches (WO97/48440, WO98/28037), or applied to the surface of the skin (transdermal or transcutaneous delivery WO98/20734, WO98/28037).

When the vaccines of the present invention are to be administered to the skin, or more specifically into the dermis, the vaccine is in a low liquid volume, particularly a volume of between about 0.05 ml and 0.2 ml.

Another suitable administration route is the subcutaneous route. Any suitable device may be used for subcutaneous delivery, for example classical needle. In one aspect of the invention, a needle-free jet injector service is used, such as that published in WO01/05453, WO01/05452, WO01/05451, WO01/32243, WO01/41840, WO01/41839, WO01/47585, WO01/56637, WO01/58512, WO01/64269, WO01/78810, WO01/91835, WO01/97884, WO02/09796, WO02/34317. In another aspect of the invention, the device is pre-filled with the liquid vaccine formulation.

Alternatively the vaccine is administered intranasally. Typically, the vaccine is administered locally to the nasopharyngeal area, e.g. without being inhaled into the lungs. It is desirable to use an intranasal delivery device which delivers the vaccine formulation to the nasopharyngeal area, without or substantially without it entering the lungs. Preferred devices for intranasal administration of the vaccines according to the invention are spray devices. Suitable commercially available nasal spray devices include Accuspray™ (Becton Dickinson).

In an embodiment, spray devices for intranasal use are devices for which the performance of the device is not dependent upon the pressure applied by the user. These devices are known as pressure threshold devices. Liquid is released from the nozzle only when a threshold pressure is applied. These devices make it easier to achieve a spray with a regular droplet size. Pressure threshold devices suitable for use with the present invention are known in the art and are described for example in WO91/13281 and EP311 863 and EP516636, incorporated herein by reference. Such devices are commercially available from Pfeiffer GmbH and are also described in Bommer, R. Pharmaceutical Technology Europe, September 1999.

In another embodiment, intranasal devices produce droplets (measured using water as the liquid) in the range 1 to 200 μm, e.g. 10 to 120 μm. Below 10 μm there is a risk of inhalation, therefore it is desirable to have no more than about 5% of droplets below 10 μm. Droplets above 120 μm do not spread as well as smaller droplets, so it is desirable to have no more than about 5% of droplets exceeding 120 μm.

Bi-dose delivery is another embodiment of an intranasal delivery system for use with the vaccines according to the invention. Bi-dose devices contain two sub-doses of a single vaccine dose, one sub-dose for administration to each nostril. Generally, the two sub-doses are present in a single chamber and the construction of the device allows the efficient delivery of a single sub-dose at a time. Alternatively, a monodose device may be used for administering the vaccines according to the invention.

A further aspect of the invention is a method of making a vaccine of the invention comprising the steps of mixing the unconjugated S. pneumoniae protein with the adjuvant composition.

Although the vaccine of the invention may be administered as a single dose, components thereof may also be co-administered together at the same time or at different times (for instance pneumococcal saccharide conjugates could be administered separately, at the same time or 1 to 2 weeks after the administration of the any bacterial protein component of the vaccine for optimal coordination of the immune responses with respect to each other). Following an initial vaccination, subjects may receive one or several booster immunisation adequately spaced.

### General

Around” or “approximately” are defined as within 10% more or less of the given figure for the purposes of the invention.

The terms “comprising”, “comprise” and “comprises” herein are intended by the inventors to be optionally substitutable with the terms “consisting of”, “consist of” and “consists of”, respectively, in every instance.

Embodiments herein relating to “vaccine compositions” of the invention are also applicable to embodiments relating to “immunogenic compositions” of the invention, and vice versa.

The term ‘Pneumococcal Histidine Triad’ is considered to be interchangeable with the term ‘Poly Histidine Triad’.

All references or patent applications cited within this patent specification are incorporated by reference herein.

In order that this invention may be better understood, the following examples are set forth. These examples are for purposes of illustration only, and are not to be construed as limiting the scope of the invention in any manner.

### EXAMPLES

### Example 1

### Production of Vaccines for Clinical Trial Study

6 Vaccines were designed:
dPly-10-AIPO₄: A vaccine comprising 10 μg of dPly adjuvanted with Aluminium phosphate.
dPly-30-AIPO₄: A vaccine comprising 30 μg of dPly adjuvanted with Aluminium phosphate.
dPly/PhtD-10-AIPO₄: A vaccine comprising 10 μg of dPly and 10 μg of PhtD adjuvanted with Aluminium phosphate.
dPly/PhtD-30-AIPO₄: A vaccine comprising 30 μg of dPly and 30 μg of PhtD adjuvanted with Aluminium phosphate.
10PCV/dPly/PhtD-10-AIPO₄: A vaccine comprising the following antigens adjuvanted to Aluminium phosphate:
1 μg capsular saccharide from serotype 1 conjugated to protein D (1-PD)
3 μg capsular saccharide from serotype 4 conjugated to protein D (4-PD)
1 μg capsular saccharide from serotype 5 conjugated to protein D (5-PD)
1 μg capsular saccharide from serotype 6B conjugated to protein D (6B-PD)
1 μg capsular saccharide from serotype 7F conjugated to protein D (7F-PD)
1 μg capsular saccharide from serotype 9V conjugated to protein D (9V-PD)
1 μg capsular saccharide from serotype 14 conjugated to protein D (14-PD)
1 μg capsular saccharide from serotype 23F conjugated to protein D (23F-PD)
3 μg capsular saccharide from serotype 18C conjugated to tetanus toxoid (18C-TT)
1 μg capsular saccharide from serotype 19F conjugated to Diphtheria Toxin (19F-DT)
10 μg dPly

### 10 μg PhtD

10PCV/dPly/PhtD-30-AIPO₄: A vaccine comprising the following antigens adjuvanted to Aluminium phosphate:
1 μg capsular saccharide from serotype 1 conjugated to protein D (1-PD)
3 μg capsular saccharide from serotype 4 conjugated to protein D (4-PD)
1 μg capsular saccharide from serotype 5 conjugated to protein D (5-PD)
1 μg capsular saccharide from serotype 6B conjugated to protein D (6B-PD)
1 μg capsular saccharide from serotype 7F conjugated to protein D (7F-PD)
1 μg capsular saccharide from serotype 9V conjugated to protein D (9V-PD)
1 μg capsular saccharide from serotype 14 conjugated to protein D (14-PD)
1 μg capsular saccharide from serotype 23F conjugated to protein D (23F-PD)
3 μg capsular saccharide from serotype 18C conjugated to tetanus toxoid (18C-TT)
1 μg capsular saccharide from serotype 19F conjugated to Diphtheria Toxin (19F-DT)
30 μg dPly

### 30 μg PhtD

10PCV: A vaccine comprising the following antigens adjuvanted to Aluminium phosphate:
1 μg capsular saccharide from serotype 1 conjugated to protein D (1-PD)
3 μg capsular saccharide from serotype 4 conjugated to protein D (4-PD)
1 μg capsular saccharide from serotype 5 conjugated to protein D (5-PD)
1 μg capsular saccharide from serotype 6B conjugated to protein D (6B-PD)
1 μg capsular saccharide from serotype 7F conjugated to protein D (7F-PD)
1 μg capsular saccharide from serotype 9V conjugated to protein D (9V-PD)
1 μg capsular saccharide from serotype 14 conjugated to protein D (14-PD)
1 μg capsular saccharide from serotype 23F conjugated to protein D (23F-PD)
3 μg capsular saccharide from serotype 18C conjugated to tetanus toxoid (18C-TT)
1 μg capsular saccharide from serotype 19F conjugated to Diphtheria Toxin (19F-DT)
30 μg dPly

### 30 μg PhtD

### Preparation of Antigens

Preparation of dPly:

Pneumococcal pneumolysin was prepared and detoxified as described in WO2004/081515 and WO2006/32499 using formaldehyde detoxification.

Expression and Purification of PhtD:

Expression OF PhtD:

The PhtD protein is a member of the pneumococcal histidine-triad (Pht) protein family characterized by the presence of histidine-triads. PhtD is a 838 aa-molecule and carries 5 histidine triads (see MedImmune WO00/37105 SEQ ID NO: 4 for amino acid sequence and SEQ ID NO: 5 for DNA sequence). PhtD also contains a proline-rich region in the middle (amino acid position 348-380). PhtD has a 20 aa-N-terminal signal sequence. Preparation and purification of PhtD is described in WO2007/071710 (see for example Example 1b).

Expression of Protein D

Protein D was expressed as described in WO2007/071710

Preparation of Conjugates

It is well known in the art how to make purified pneumococcal polysaccharides. For the purposes of these examples the polysaccharides were made essentially as described in EP072513 or by closely-related methods. Before conjugation the polysaccharides may be sized by microfluidisation as described below.

The activation and coupling conditions are specific for each polysaccharide. These are given in Table 1. Sized polysaccharide (except for 6B and 23F) was dissolved in NaCl 2M, NaCl 0.2M or in water for injection (WFI). The optimal polysaccharide concentration was evaluated for all the serotypes. All serotypes except serotype 18C were conjugated directly to the carrier protein as detailed below.

From a 100 mg/ml stock solution in acetonitrile or acetonitrile/water (50%/50%) solution, CDAP (1-cyano-4-dimethylamino pyridinium tetrafluoroborate) (CDAP/PS ratio 0.5-1.5 mg/mg PS) was added to the polysaccharide solution. 1.5 minute later, 0.2M-0.3M NaOH was added to obtain the specific activation pH. The activation of the polysaccharide was performed at this pH for 3 minutes at 25° C. Purified protein (protein D, CRM197 or DT) (the quantity depends on the initial PS/carrier protein ratio) was added to the activated polysaccharide and the coupling reaction was performed at the specific pH for up to 2 hour (depending upon serotype) under pH regulation. In order to quench un-reacted cyanate ester groups, a 2M glycine solution was then added to the mixture. The pH was adjusted to the quenching pH (pH 9.0). The solution was stirred for 30 minutes at 25° C. and then incubated overnight at 2-8° C. with continuous slow stirring.

Preparation of 18C:

18C was linked to the carrier protein via a linker—Adipic acid dihydrazide (ADH) Polysaccharide serotype 18C was microfluidized before conjugation.

Derivatization of Tetanus Toxoid with EDAC (1-ethyl-3-(3-dimethylaminopropyl)carbodiimide)

For derivatization of the tetanus toxoid, purified TT was diluted at 25 mg/ml in 0.2M NaCl and the ADH spacer was added in order to reach a final concentration of 0.2M. When the dissolution of the spacer was complete, the pH was adjusted to 6.2. EDAC (1-ethyl-3-(3-dimethyl-aminopropyl)carbodiimide) was then added to reach a final concentration of 0.02M and the mixture was stirred for 1 hour under pH regulation. The reaction of condensation was stopped by increasing pH up to 9.0 for at least 30 minutes at 25° C.

Derivatized TT was then diafiltrated (10 kDa CO membrane) in order to remove residual ADH and EDAC reagent.

TT_{AH} (tetanus toxoid conjugated to an ADH linker) bulk was finally sterile filtered until coupling step and stored at −70° C.

Chemical coupling of TT_{AH} to PS 18C

Details of the conjugation parameters can be found in Table 1.

2 grams of microfluidized PS were diluted at the defined concentration in water and adjusted to 2M NaCl by NaCl powder addition.

CDAP solution (100 mg/ml freshly prepared in 50/50 v/v acetonitrile/WFI) was added to reach the appropriate CDAP/PS ratio.

The pH was raised up to the activation pH 9.0 by the addition of 0.3M NaOH and was stabilised at this pH until addition of TT_{AH}.

After 3 minutes, derivatized TT_{AH} (20 mg/ml in 0.2 M NaCl) was added to reach a ratio TT_{AH}/PS of 2; the pH was regulated to the coupling pH 9.0. The solution was left one hour under pH regulation.

For quenching, a 2M glycine solution, was added to the mixture PS/TT_{AH}/CDAP.

The pH was adjusted to the quenching pH (pH 9.0).

The solution was stirred for 30 min at 25° C., and then overnight at 2-8° C. with continuous slow stirring.

### Specific Activation/Coupling/Quenching Conditions of S. Pneumoniae Capsular Saccharide-Protein D/TT/DT/PhtD/Ply Conjugates

Where “μfluid” appears in a row header, it indicates that the saccharide was sized by microfluidisation before conjugation. Sizes of saccharides following microfluidisation are given in table 2.

TABLE 1
Specific activation/coupling/quenching conditions of S. pneumoniae
capsular saccharide-Protein D/TT/DT/CRM197 conjugates

| | Serotype | | | | | |
|---|---|---|---|---|---|---|
| | 1 | 4 | 5 | 6A | | 7F |
| | μfluid | μfluid | μfluid | μfluid | 6B | μfluid |
| PS | 2.27 | 2.37 | 7.1 | 10 | 5.0 | 5.0 |
| conc.(mg/ml) | | | | | | |
| PS | WFI | WFI | WFI | NaCl 2M | NaCl 2M | NaCl 2M |
| dissolution | | | | | | |
| Carrier | 10.0 | 10.0 | 5.0 | 10 | 5.0 | 10.0 |
| conc.(mg/ml) | PD | PD | PD | CRM197 | PD | PD |
| Initial | 1.65/1 | 1.60/1 | 1/1 | 1.5/1 | 1.1/1 | 1.2/1 |
| PROT/PS | | | | | | |
| Ratio (w/w) | | | | | | |
| CDAP conc. | 0.55 | 0.55 | 0.79 | 1.0 | 0.83 | 0.75 |
| (mg/mg PS) | | | | | | |
| pHₐ = pH_{c} = pH_{q} | 9.0/9.0/9.0 | 9.5/9.5/9.0 | 9.0/9.0/9.0 | 9.5/9.5/9.0 | 9.5/9.5/9.0 | 9.5/9.5/9.0 |
| | Serotype | | | | | |
| | 9V | 14 | 18C | 19A | 19F | |
| | μfluid | μfluid | μfluid | μfluid | μfluid | 23F |
| PS | 5.0 | 5.0 | 4.5 | 15.0 | 9.0 | 2.38 |
| conc.(mg/ml) | | | | | | |
| PS | NaCl 2M | NaCl 2M | NaCl 2M | NaCl 2M | NaCl 2M | NaCl 2M |
| dissolution | | | | | | |
| Carrier | 10.0 | 10.0 | 20.0 (TT) | 15.0 | 20.0 | 5.0 |
| protein | | | | (CRM197) | (DT) | |
| conc.(mg/ml) | | | | | | |
| Initial carrier | 1.2/1 | 1.2/1 | 2/1 | 1.5/1 | 1.5/1 | 1/1 |
| protein/PS | | | | | | |
| Ratio (w/w) | | | | | | |
| CDAP conc. | 0.50 | 0.75 | 0.75 | 1.5 | 1.5 | 0.79 |
| (mg/mg PS) | | | | | | |
| pHₐ = pH_{c} = pH_{q} | 9.5/9.5/9.0 | 9.5/9.5/9.0 | 9.0/9.0/9.0 | 9.0/9.0/9.0 | 9.0/9.0/9.0 | 9.5/9.5/9.0 |

Note:
pHa, c, q corresponds to the pH for activation, coupling and quenching, respectively

Purification of the Conjugates:

The conjugates were purified by gel filtration using a Sephacryl S400HR gel filtration column equilibrated with 0.15M NaCl (except S500HR was used as buffer for 18C and 20 mM acetate containing 1.15MNaCl pH6.2 was used for 19A) to remove small molecules (including DMAP) and unconjugated saccharide and protein. Based on the different molecular sizes of the reaction components, PS-PD, PS-TT, PS-CRM197 or PS-DT conjugates are eluted first, followed by free PS, then by free protein carrier and finally DMAP and other salts (NaCl, glycine).

Fractions containing conjugates are detected by UV_{280 nm}. Fractions are pooled according to their Kd, sterile filtered (0.22 μm) and stored at +2-8° C. The PS/Protein ratios in the conjugate preparations were determined.

### Example 2

### Clinical Trial to Study Efficacy of Vaccines Comprising High and Low Doses of dPly and PhtD Carried Out in Adults

A clinical trial was carried out using seven parallel groups;

1. dPly-10-AIPO₄ group (‘Ply-10’ in result tables and figures): subjects receiving the dPly-10-AIPO₄ vaccine as described in example 1 (formulated with 167 μg AIPO₄, 150 mM NaCl and 0.78 mM PO₄ buffer).
2. dPly-30-AIPO₄ group (‘Ply-30’ in result tables and figures): subjects receiving the dPly-30-AIPO₄ vaccine as described in example 1 (formulated with 500 AIPO₄, 150 nM NaCl and 0.78 mM PO₄ buffer).
3. dPly/PhtD-10-AIPO₄ group (‘PIPh-10’ in result tables and figures): subjects receiving the dPly/PhtD-10-AIPO₄ vaccine as described in example 1 (formulated with 500 μg AIPO₄, 150 mM NaCl and 0.72 mM PO₄ buffer).
4. dPly/PhtD-30-AIPO₄ group (‘PIPh-30’ in result tables and figures): subjects receiving the dPly/PhtD-30-AIPO₄ vaccine as described in example 1 (formulated with 500 μg AIPO₄, 150 mM NaCl and 0.86 mM PO₄ buffer).
5. 10PCV/dPly/PhtD-10-AIPO₄ group (‘10vPP10’ in result tables and figures): subjects receiving the 10PCV/dPly/PhtD-10-AIPO₄ vaccine as described in example 1 (formulated with 500 μg AIPO₄, 150 mM NaCl and 0.72 mM PO₄ buffer).
6. 10PCV/dPly/PhtD-30-AIPO₄ group (‘10vPP30’ in result tables and figures): subjects receiving the 10PCV/dPly/PhtD-30-AIPO₄ vaccine as described in example 1 (formulated with 500 μg AIPO₄, 150 mM NaCl and 0.86 mM PO₄ buffer).
7. 23PPV Comparator group (‘23PPV’ in result tables and figures): subjects receiving one dose of a commercially available licensed 23-valent c Polysaccharide Vaccine (23PPV) named Pneumovax 23™ at Day 0 (Month 0) followed by placebo at Day 60 (Month 2).

Subjects in all groups received 2 doses of the respective study vaccines, one at Day 0 (Month 0) and one at Day 60 (Month 2); the 23PPV Comparator group received the licensed 23PPV vaccine at Day 0 (Month 0) and a placebo at Day 60 (Month 2). Vaccines were administered as an intramuscular injection in the non dominant deltoid. Subjects were healthy males or females between, and including, 18 and 40 years old at the time of the first vaccination.

Safety/Reactogenicity Methods

Incidence of solicited and/or unsolicited local and general AEs (adverse events) during the 31-day post-vaccination follow-up period was calculated with 95% Cl (confidance interval), after each vaccine dose and overall, according to the type of symptom, the intensity and relationship to vaccination. Incidence of each local and each general solicited symptom reported during the 7-day post-vaccination follow-up period was calculated with 95% Cl, after each vaccine dose and overall, according to the type of symptom, the intensity and relationship to vaccination. The percentages of subjects/doses with an unsolicited symptom reported within the 31-day post-vaccination follow-up period were summarized according to the Medical Dictionary for Regulatory Activities (MedDRA), with 95% Cl (confidance interval) according to the intensity and relationship to vaccination. Prevalence of concomitant antipyretic/medication during the 7-day post-vaccination follow-up period and during the 31-day post-vaccination follow-up period was computed with 95% Cl, after each vaccine dose and overall. Serious adverse events (SAEs) and withdrawals due to adverse event(s) reported during the entire study period were described in detail. For each haematology and biochemistry parameter, the number and percentage of subjects with levels below, within and above normal ranges were tabulated per study group, at each assessed time point. The grading and the change in grading from baseline (baseline: day 0 assessment) was summarized per study group, at each assessed time point. For each urine parameter, the grading and the change in grading from baseline (baseline: day 0 assessment) was summarized per study group, at each assessed time point. pH quantified in urinalysis over time was displayed per study group.

Laboratory Assays and Time Points for Immunogenicity Evaluation

For all subjects, an approximately 20 mL sample of whole venous blood was collected for serology at Visit 1 [Pre], Visit 4 [(PI(D30) (post dose I at day 30)] and Visit 8 [(PII(D90) (post dose II at day 90)]. After blood centrifugation and serum separation, samples were stored at −20° C. until collection by the sponsor. The aliquots of serum (approximately 10 mL) were sent to GSK Biologicals for the serological tests described in the following paragraphs.
Opsonophagocytic activity for antibodies against each of the 10 pneumococcal serotypes (antibodies to 1, 4, 5, 6B, 7F, 9V, 14, 18C, 19F, 23F) was measured by a killing-assay using a HL 60 cell line. The results were presented as the dilution of serum (opsonic titre) able to sustain 50% killing of live pneumococci under the assay conditions. The cut-off of the assay was an opsonic titre of 8.
Anti-dPly and anti-PhtD antibodies were quantified using an ELISA (enzyme linked immunosorbent assay).

A sample ELISA protocol is described below:

Microtiter plates are coated with purified serotype specific pneumococcal PS (polysaccharide) and mixed with methylated human serum albumin (0.5 to 7.5 μg/ml, depending on the PS). Serum samples are diluted in adsorption buffer (phosphate-buffered saline [PBS], 10% fetal calf serum [FCS], 0.1% polyvinyl alcohol) containing 10 μg/ml of the CWPS (capsular wall polysaccharide) (SSI) and 2 μg/ml of PS of serotype 22F (ATCC) and are incubated overnight at 4° C. The 89-SF reference serum sample (FDA) (89-SF is the number of the reference serum calibrated by the World Health Organisation) is diluted in the same adsorption buffer but with 10 μg/ml of CWPS only, and the mixture is incubated overnight at 4° C. An internal reference serum sample, calibrated against standard serum sample 89-SF, is included in every plate. Unlike the working standard serum sample, in line with the WHO recommendations, the 89-SF reference serum sample is preadsorbed only with CWPS and not with 22F because the values for the anti-PS concentrations of the 89-SF reference serum sample are assigned without the use of 22F PS. Bound antibodies are detected by using alkaline phosphatase-conjugated anti-human IgG.

A sample OPA (opsonophagocytosis assay) is described below:

The OPA assay is performed as follows: serum samples are heated for 40 min at 56° C. to inactivate any remaining endogenous complement. Twenty-five microlitres aliquots of each 1:2 diluted serum sample is two-fold serially diluted in 25 μl OPA buffer (Hank's Balanced salt solution or HBSS −14.4% inactivated FBS (fetal bovine serum)) per well of a 96-well round bottom microtitre plate (Nalge Nunc International, Rochester, N.Y., USA). Two positive control samples with known opsonophagocytic titre for the specific pneumococcal serotype are run on each assay plate to determine the assay validity. In one column no serum sample is added to estimate the average CFU (colony forming units) which corresponds to 0% killing in the absence of opsonophagocytic antibodies. Subsequently, 25 μl of a mixture of activated HL-60 cells (1×107 cells/ml), freshly thawed pneumococcal working seed and freshly thawed baby rabbit complement in an e.g. 4/2/2.82 ratio (volume/volume/volume (v/v/v); the ratio may vary depending on the lot of complement) are added to the diluted sera to yield a final volume of 50 μl. As a result, final serial dilutions of serum samples are 1:8-1:1024. The assay plate is incubated for 2 h at 37° C. with orbital shaking (210 rpm) to promote the phagocytic process. The reaction is stopped by laying the microplate on ice for at least 1 min. A 20 μl aliquot of each well of the plate is then transferred into the corresponding well of a 96-well flat bottom microplate (Nalge Nunc International) and 100 μl of Todd-Hewitt Broth-0.9% agar is added to each well. After overnight incubation at 37° C. and 5% CO₂, pneumococcal colonies appearing in the agar are counted using an automated image analysis system (KS 400, Zeiss, Oberkochen, Germany). Eight wells without serum sample are used as bacterial controls to determine the number of pneumococci per well. The mean number of CFU of the control wells is determined and used for the calculation of the killing activity for each serum sample. The OPA titre for the serum samples is determined by the reciprocal dilution of serum able to facilitate 50% killing of the pneumococci. The opsonophagocytic titre is calculated by using a 4-parameter curve fit analysis. Each sample titre was adjusted through an adjustment factor based on the average ratio of actually measured titre/known titre of two positive control samples included in each assay. The cut-off OPA titre for all serotypes is a reciprocal serum dilution of 8. Serum samples with OPA titres <8 are assigned a titre of 4 for the purpose of data analysis.

A sample anti-PhtD and anti-Ply ELISA is described below

Plates are coated overnight at 4° C. with 100 μl per well of 2 μg/ml of PhtD (1021 μg/ml) in PBS, 3 μg/ml of Ply (3675 μg/ml) in TR034 (Na 50 mM ph 9.5).

The plates are then washed three times with TR 112 (Na2K 39.65 mM-NaCl 1.73 M-Polysorbate 20 0.275% 5P/V° pH 6.6-6.8).

After the first wash, the plates are saturated for 1 hour at RT with 200 μl/wells of diluant buffer (PBS without Ca and Mg-BSA 1% for PhtD) or BSA 5% for Ply-Polysorbate 20 0.1%-ProClin 300™ 0.2% pH 7.0) with shaking.

After a second wash (same buffer) 100 μl of sera are added to microwells at the dilution 1/100, 1/1000 and 1/10000 in the diluant buffer.

The plates are incubated for 1 hour at room temperature with shaking. After 5 washes, the plates are incubated with a goat anti-human IgG Fc antibody conjugated to peroxydase, diluted 1/20000 for PhtD and 1/12500 for Ply (100 μl per well) at room temperature for 1 hour with shaking. Plates are washed as above and 100 μl of the TMB substrate conjugate is added to each well for 10 min in darkness.

The reaction is stopped by addition of H2SO4 (0.18M) 100 μl and the absorbance is read at 450 nm and 650 nm.

Immunogenicity

Immunogenicity analysis was performed on the according to protocol (ATP) cohort for immunogenicity. No second analysis on the Total vaccinated cohort was performed as less than 5% of subjects were excluded from the ATP cohort for immunogenicity. Sera were analysed for the presence of antibodies against dPly, PhtD, and Streptococcus pneumoniae capsular saccharide serotypes 1, 4, 5, 6B, 7F, 9V, 14, 18C, 19F and 23F.

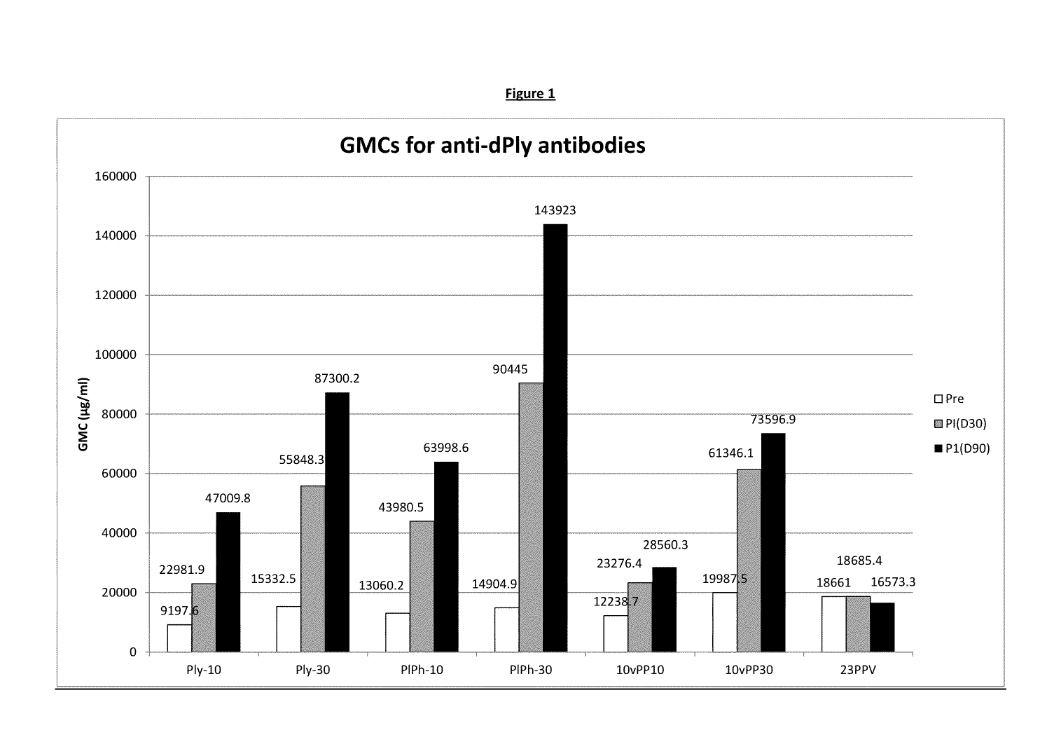

The seropositivity rates and GMCs for anti-Ply antibodies are described in table 1 below, these data are illustrated in FIG. 1:

TABLE 1
Seropositivity rates and GMCs for anti-Ply antibodies (ATP cohort for immunogenicity)

| | | | | ≧599 LU/mL | | | | GMC | | |
|---|---|---|---|---|---|---|---|---|---|---|
| | | | | | | 95% CI | | | 95% CI | |
| Antibody | Group | Timing | N | n | % | LL | UL | value | LL | UL |
| Anti- | Ply-10 | PRE | 10 | 10 | 100 | 69.2 | 100 | 9197.6 | 5993.2 | 14115.4 |
| dPly | | PI(D30) | 10 | 10 | 100 | 69.2 | 100 | 22981.9 | 15630.8 | 33790.3 |
| | | PII(D90) | 10 | 10 | 100 | 69.2 | 100 | 47009.8 | 30960.2 | 71379.4 |
| | Ply-30 | PRE | 24 | 24 | 100 | 85.8 | 100 | 15332.5 | 10750.7 | 21866.9 |
| | | PI(D30) | 24 | 24 | 100 | 85.8 | 100 | 55848.3 | 37181.4 | 83886.8 |
| | | PII(D90) | 21 | 21 | 100 | 83.9 | 100 | 87300.2 | 58201.8 | 130947 |
| | PlPh-10 | PRE | 24 | 24 | 100 | 85.8 | 100 | 13060.2 | 9621.1 | 17728.5 |
| | | PI(D30) | 24 | 24 | 100 | 85.8 | 100 | 43980.5 | 31936 | 60567.7 |
| | | PII(D90) | 23 | 23 | 100 | 85.2 | 100 | 63998.6 | 48405.7 | 84614.4 |
| | PlPh-30 | PRE | 23 | 23 | 100 | 85.2 | 100 | 14904.9 | 11128.3 | 19963 |
| | | PI(D30) | 24 | 24 | 100 | 85.8 | 100 | 90445 | 62893.9 | 130065 |
| | | PII(D90) | 24 | 24 | 100 | 85.8 | 100 | 143923 | 106150 | 195138 |
| | 10vPP10 | PRE | 24 | 24 | 100 | 85.8 | 100 | 12238.7 | 8648.1 | 17319.9 |
| | | PI(D30) | 24 | 24 | 100 | 85.8 | 100 | 23276.4 | 16926.3 | 32008.9 |
| | | PII(D90) | 24 | 24 | 100 | 85.8 | 100 | 28560.3 | 21331.4 | 38239.1 |
| | 10vPP30 | PRE | 24 | 24 | 100 | 85.8 | 100 | 19987.5 | 15149.1 | 26371.2 |
| | | PI(D30) | 24 | 24 | 100 | 85.8 | 100 | 61346.1 | 46049.1 | 81724.5 |
| | | PII(D90) | 21 | 21 | 100 | 83.9 | 100 | 73596.9 | 52250.3 | 103665 |
| | 23PPV | PRE | 24 | 24 | 100 | 85.8 | 100 | 18661 | 12726.5 | 27363 |
| | | PI(D30) | 24 | 24 | 100 | 85.8 | 100 | 18685.4 | 12612.1 | 27683.4 |
| | | PII(D90) | 23 | 23 | 100 | 85.2 | 100 | 16573.3 | 11007.1 | 24954.2 |

A seropositive subject was defined as a subject with antibody concentration ≧0.05 μg/mL.
GMC = geometric mean antibody concentration calculated on all subjects
N = number of subjects with available results
n/% = number/percentage of subjects with concentration within the specified range
95% CI = 95% confidence interval; LL = lower limit, UL = upper limit
PRE = pre-vaccination blood sample
PI(D30) = blood sample taken post dose I at day 30
PII (D90) = blood sample taken post dose II at day 90

An increase in the anti-dPly antibodies GMCs was observed following each vaccination in all groups, except for the 23PPV group that did not receive dPly. The anti-dPly antibody GMCs tended to be higher in the PIPh-10 and PIPh-30 groups compared to the Ply-10 and Ply-30 groups respectively this is unexpected as one might expect antigen interference between the PhtD and dPly. The highest anti-dPly antibody GMCs were observed for the formulation containing 30 μg of dPly and PhtD proteins (i.e PIPh-30 and 10vPP30 groups).

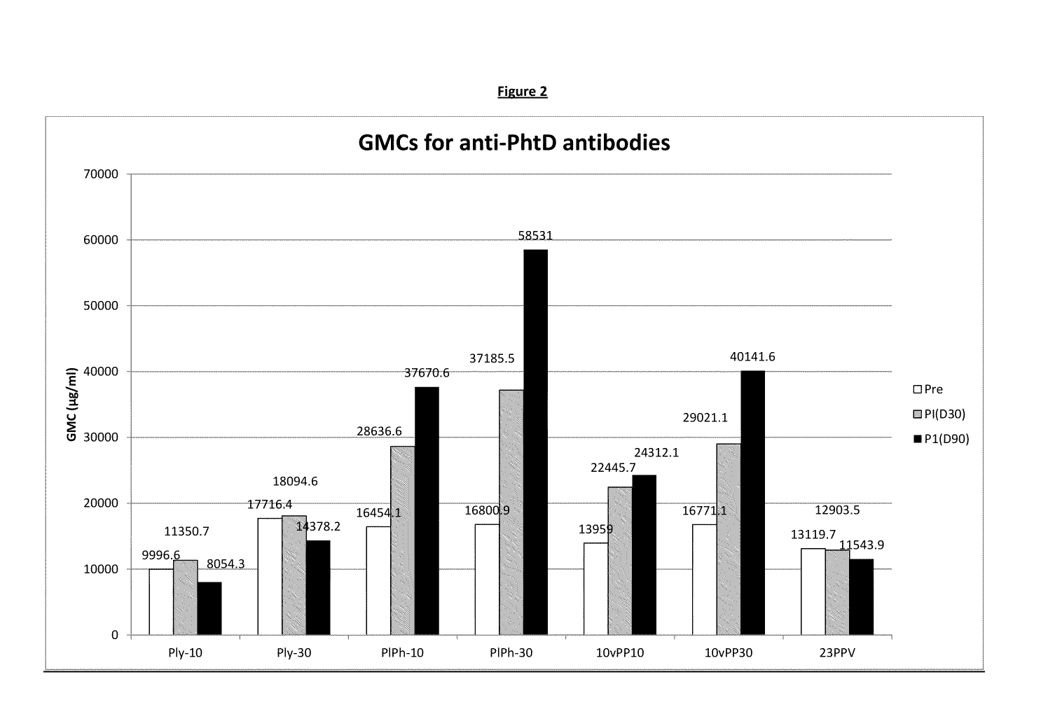

The seropositivity rates and GMCs for anti-PhtD antibodies (ATP cohort of immunogenicity) are described in table 2 below and illustrated in FIG. 2:

TABLE 2
Seropositivity rates and GMCs for anti-PhtD antibodies (ATP cohort for immunogenicity)

| | | | | ≧391 LU/mL | | | | GMC | | |
|---|---|---|---|---|---|---|---|---|---|---|
| | | | | | | 95% CI | | | 95% CI | |
| Antibody | Group | Timing | N | n | % | LL | UL | value | LL | UL |
| Anti- | Ply-10 | PRE | 10 | 10 | 100 | 69.2 | 100 | 9996.6 | 6608.7 | 15121.2 |
| PhtD | | PI(D30) | 10 | 10 | 100 | 69.2 | 100 | 11350.7 | 6928.5 | 18595.5 |
| | | PII(D90) | 10 | 10 | 100 | 69.2 | 100 | 8054.3 | 5005.4 | 12960.5 |
| | Ply-30 | PRE | 24 | 24 | 100 | 85.8 | 100 | 17716.4 | 13160.7 | 23849.1 |
| | | PI(D30) | 24 | 24 | 100 | 85.8 | 100 | 18094.6 | 13270.3 | 24672.7 |
| | | PII(D90) | 21 | 21 | 100 | 83.9 | 100 | 14378.2 | 10119.6 | 20428.9 |
| | PlPh-10 | PRE | 24 | 24 | 100 | 85.8 | 100 | 16454.1 | 11491.1 | 23560.6 |
| | | PI(D30) | 24 | 24 | 100 | 85.8 | 100 | 28636.6 | 20777.7 | 39468.1 |
| | | PII(D90) | 23 | 23 | 100 | 85.2 | 100 | 37670.6 | 29311.2 | 48414 |
| | PlPh-30 | PRE | 23 | 23 | 100 | 85.2 | 100 | 16800.9 | 13270.5 | 21270.5 |
| | | PI(D30) | 24 | 24 | 100 | 85.8 | 100 | 37185.5 | 28847.4 | 47933.7 |
| | | PII(D90) | 24 | 24 | 100 | 85.8 | 100 | 58531 | 49363.7 | 69400.8 |
| | 10vPP10 | PRE | 24 | 24 | 100 | 85.8 | 100 | 13959 | 10649.8 | 18296.6 |
| | | PI(D30) | 24 | 24 | 100 | 85.8 | 100 | 22445.7 | 17793.5 | 28314.3 |
| | | PII(D90) | 24 | 24 | 100 | 85.8 | 100 | 24312.1 | 18800.7 | 31439.3 |
| | 10vPP30 | PRE | 24 | 24 | 100 | 85.8 | 100 | 16771.1 | 11893.3 | 23649.5 |
| | | PI(D30) | 24 | 24 | 100 | 85.8 | 100 | 29021.1 | 20873.4 | 40349 |
| | | PII(D90) | 21 | 21 | 100 | 83.9 | 100 | 40141.6 | 29095.1 | 55382.1 |
| | 23PPV | PRE | 24 | 24 | 100 | 85.8 | 100 | 13119.7 | 10035.1 | 17152.5 |
| | | PI(D30) | 24 | 24 | 100 | 85.8 | 100 | 12903.5 | 9567.7 | 17402.4 |
| | | PII(D90) | 23 | 23 | 100 | 85.2 | 100 | 11543.9 | 8457.8 | 15756.1 |

An increase in the anti-PhtD antibody GMCs was observed with each dose except for the Ply-10, Ply-30 and 23PPV groups that did not receive PhtD. Anti-PhtD antibody GMCs post-dose 1 and post dose-2 tended to be lower in the 10vPP10 and 10vPP30 groups compared to the PIPh-10 and PIPh-30 groups, respectively. The highest anti-PhtD antibody GMCs were observed for the formulations containing 30 μg of dPly and PhtD proteins (i.e PIPh-30 and 10vPP30 groups).

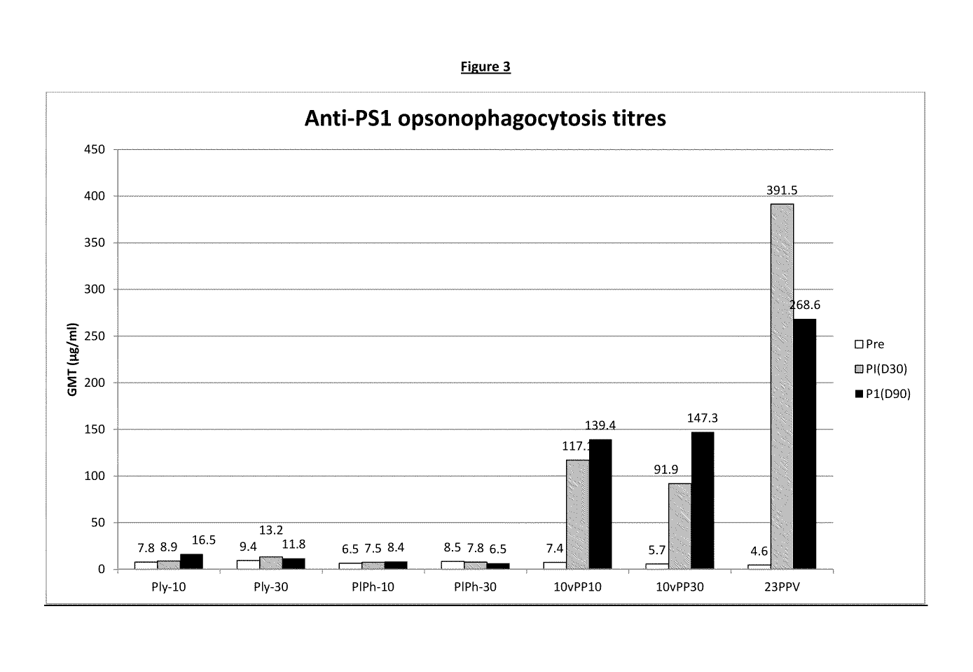

Seropositivity rates and GMTs for serotype 1 antibodies are described in Table 3 below, these data are illustrated in FIG. 3:

TABLE 3
Seropositivity rates and GMTs for anti-PS1
antibodies (ATP cohort for immunogenicity)

| | | | | | | 95% CI | | | 95% CI | |
|---|---|---|---|---|---|---|---|---|---|---|
| Antibody | Group | Timing | N | n | % | LL | UL | value | LL | UL |
| OPSONO- | Ply-10 | PRE | 10 | 3 | 30 | 6.7 | 65.2 | 7.8 | 3.6 | 16.8 |
| 1 | | PI(D30) | 10 | 5 | 50 | 18.7 | 81.3 | 8.9 | 4.8 | 16.5 |
| | | PII(D90) | 10 | 6 | 60 | 26.2 | 87.8 | 16.5 | 5.8 | 46.7 |
| | Ply-30 | PRE | 21 | 6 | 28.6 | 11.3 | 52.2 | 9.4 | 4.8 | 18.3 |
| | | PI(D30) | 22 | 13 | 59.1 | 36.4 | 79.3 | 13.2 | 7.7 | 22.7 |
| | | PII(D90) | 21 | 11 | 52.4 | 29.8 | 74.3 | 11.8 | 6.9 | 20 |
| | PlPh-10 | PRE | 23 | 3 | 13 | 2.8 | 33.6 | 6.5 | 3.5 | 12 |
| | | PI(D30) | 23 | 5 | 21.7 | 7.5 | 43.7 | 7.5 | 4 | 14.3 |
| | | PII(D90) | 22 | 6 | 27.3 | 10.7 | 50.2 | 8.4 | 4.4 | 16.4 |
| | PlPh-30 | PRE | 20 | 8 | 40 | 19.1 | 63.9 | 8.5 | 5.3 | 13.4 |
| | | PI(D30) | 24 | 9 | 37.5 | 18.8 | 59.4 | 7.8 | 5.4 | 11.3 |
| | | PII(D90) | 24 | 6 | 25 | 9.8 | 46.7 | 6.5 | 4.5 | 9.5 |
| | 10vPP10 | PRE | 21 | 5 | 23.8 | 8.2 | 47.2 | 7.4 | 4.2 | 13 |
| | | PI(D30) | 24 | 23 | 95.8 | 78.9 | 99.9 | 117.1 | 67.7 | 202.5 |
| | | PII(D90) | 24 | 23 | 95.8 | 78.9 | 99.9 | 139.4 | 83.8 | 231.9 |
| | 10vPP30 | PRE | 22 | 4 | 18.2 | 5.2 | 40.3 | 5.7 | 4 | 8 |
| | | PI(D30) | 23 | 22 | 95.7 | 78.1 | 99.9 | 91.9 | 56 | 150.9 |
| | | PII(D90) | 21 | 21 | 100 | 83.9 | 100 | 147.3 | 98.7 | 219.8 |
| | 23PPV | PRE | 21 | 2 | 9.5 | 1.2 | 30.4 | 4.6 | 3.8 | 5.7 |
| | | PI(D30) | 24 | 24 | 100 | 85.8 | 100 | 391.5 | 193.7 | 791.4 |
| | | PII(D90) | 23 | 22 | 95.7 | 78.1 | 99.9 | 268.6 | 129 | 559.2 |

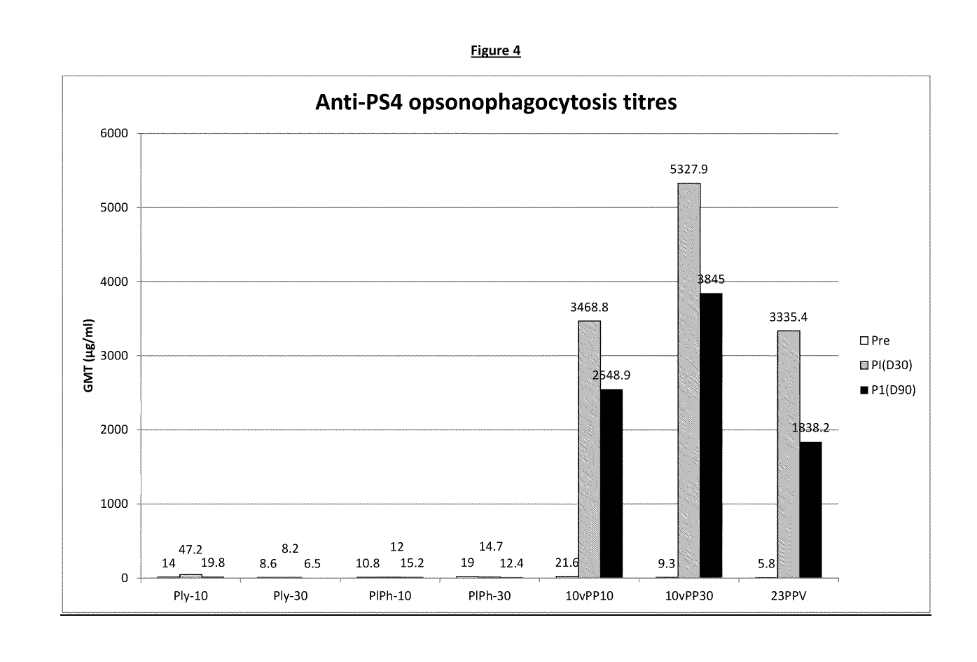

Seropositivity rates and OPA (opsonophagocytosis) GMTs for serotype 4 antibodies are described in Table 4 below, these data are illustrated in FIG. 4:

TABLE 4
Seropositivity rates and GMTs for anti-PS4 antibodies (ATP cohort for immunogenicity)

| | | | | | | 95% CI | | | 95% CI | |
|---|---|---|---|---|---|---|---|---|---|---|
| Antibody | Group | Timing | N | n | % | LL | UL | value | LL | UL |
| OPSONO- | Ply-10 | PRE | 3 | 1 | 33.3 | 0.8 | 90.6 | 14 | 0.1 | 3085.3 |
| 4 | | PI(D30) | 7 | 4 | 57.1 | 18.4 | 90.1 | 47.2 | 5.2 | 428.2 |
| | | PII(D90) | 8 | 3 | 37.5 | 8.5 | 75.5 | 19.8 | 3.1 | 126.1 |
| | Ply-30 | PRE | 17 | 3 | 17.6 | 3.8 | 43.4 | 8.6 | 3.6 | 20.5 |
| | | PI(D30) | 18 | 3 | 16.7 | 3.6 | 41.4 | 8.2 | 3.6 | 19 |
| | | PII(D90) | 21 | 2 | 9.5 | 1.2 | 30.4 | 6.5 | 3.2 | 13 |
| | PlPh-10 | PRE | 18 | 4 | 22.2 | 6.4 | 47.6 | 10.8 | 4.1 | 28.4 |
| | | PI(D30) | 21 | 5 | 23.8 | 8.2 | 47.2 | 12 | 4.8 | 30.3 |
| | | PII(D90) | 22 | 6 | 27.3 | 10.7 | 50.2 | 15.2 | 5.6 | 41.6 |
| | PlPh-30 | PRE | 15 | 5 | 33.3 | 11.8 | 61.6 | 19 | 5.4 | 67.6 |
| | | PI(D30) | 20 | 5 | 25 | 8.7 | 49.1 | 14.7 | 5 | 43.4 |
| | | PII(D90) | 22 | 5 | 22.7 | 7.8 | 45.4 | 12.4 | 4.8 | 32.2 |
| | 10vPP10 | PRE | 15 | 6 | 40 | 16.3 | 67.7 | 21.6 | 6.2 | 75.5 |
| | | PI(D30) | 24 | 24 | 100 | 85.8 | 100 | 3468.8 | 2091.4 | 5753.3 |
| | | PII(D90) | 23 | 23 | 100 | 85.2 | 100 | 2548.9 | 1735.9 | 3742.7 |
| | 10vPP30 | PRE | 17 | 3 | 17.6 | 3.8 | 43.4 | 9.3 | 3.5 | 24.8 |
| | | PI(D30) | 24 | 24 | 100 | 85.8 | 100 | 5327.9 | 3480.7 | 8155.3 |
| | | PII(D90) | 21 | 21 | 100 | 83.9 | 100 | 3845 | 2573.6 | 5744.6 |
| | 23PPV | PRE | 16 | 1 | 6.3 | 0.2 | 30.2 | 5.8 | 2.6 | 12.7 |
| | | PI(D30) | 24 | 24 | 100 | 85.8 | 100 | 3335.4 | 1902.8 | 5846.6 |
| | | PII(D90) | 23 | 22 | 95.7 | 78.1 | 99.9 | 1838.2 | 855.4 | 3950.4 |

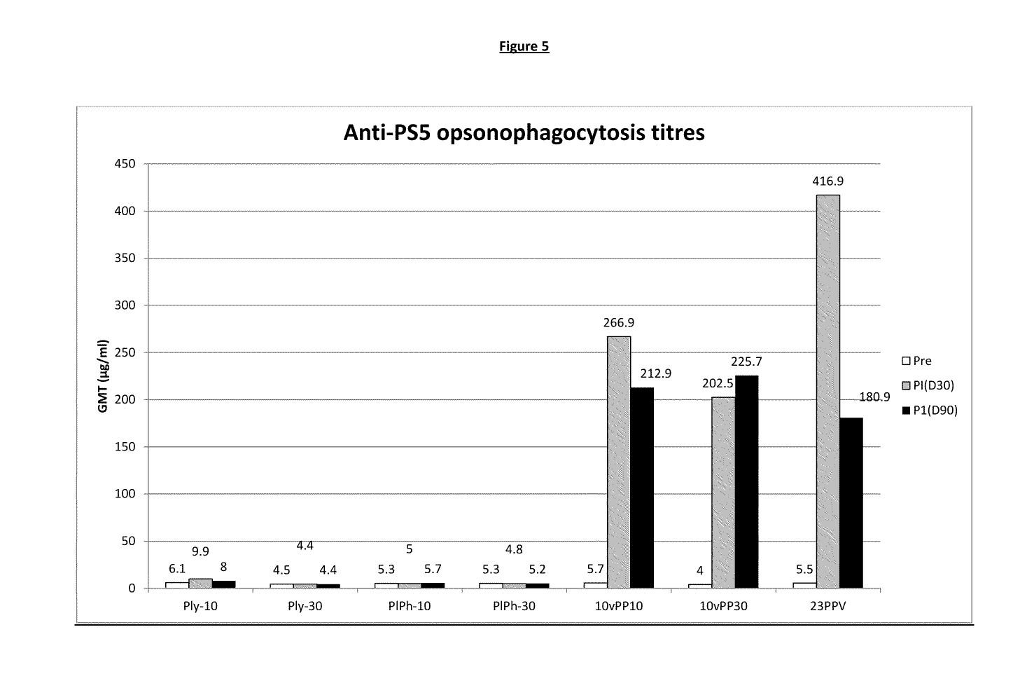

Seropositivity rates and OPA (opsonophagocytosis) GMTs for serotype 5 antibodies are described in Table 5 below, these data are illustrated in FIG. 5:

TABLE 5
Seropositivity rates and GMTs for anti-PS5
antibodies (ATP cohort for immunogenicity)

| | | | | ≧8 | | | | GMT | | |
|---|---|---|---|---|---|---|---|---|---|---|
| | | | | | | 95% CI | | | 95% CI | |
| Antibody | Group | Timing | N | n | % | LL | UL | value | LL | UL |
| OPSONO- | Ply-10 | PRE | 7 | 1 | 14.3 | 0.4 | 57.9 | 6.1 | 2.2 | 17.1 |
| 5 | | PI(D30) | 10 | 4 | 40 | 12.2 | 73.8 | 9.9 | 4 | 24.2 |
| | | PII(D90) | 10 | 4 | 40 | 12.2 | 73.8 | 8 | 3.8 | 16.9 |
| | Ply-30 | PRE | 22 | 2 | 9.1 | 1.1 | 29.2 | 4.5 | 3.8 | 5.4 |
| | | PI(D30) | 21 | 1 | 4.8 | 0.1 | 23.8 | 4.4 | 3.6 | 5.4 |
| | | PII(D90) | 21 | 2 | 9.5 | 1.2 | 30.4 | 4.4 | 3.8 | 5 |
| | PlPh-10 | PRE | 23 | 3 | 13 | 2.8 | 33.6 | 5.3 | 3.7 | 7.5 |
| | | PI(D30) | 23 | 2 | 8.7 | 1.1 | 28 | 5 | 3.6 | 7.1 |
| | | PII(D90) | 23 | 4 | 17.4 | 5 | 38.8 | 5.7 | 4 | 8.2 |
| | PlPh-30 | PRE | 23 | 5 | 21.7 | 7.5 | 43.7 | 5.3 | 4.2 | 6.7 |
| | | PI(D30) | 23 | 3 | 13 | 2.8 | 33.6 | 4.8 | 3.9 | 6 |
| | | PII(D90) | 24 | 5 | 20.8 | 7.1 | 42.2 | 5.2 | 4.1 | 6.5 |
| | 10vPP10 | PRE | 24 | 4 | 16.7 | 4.7 | 37.4 | 5.7 | 4 | 8.1 |
| | | PI(D30) | 23 | 22 | 95.7 | 78.1 | 99.9 | 266.9 | 125 | 569.8 |
| | | PII(D90) | 24 | 23 | 95.8 | 78.9 | 99.9 | 212.9 | 111.2 | 407.6 |
| | 10vPP30 | PRE | 23 | 0 | 0 | 0 | 14.8 | 4 | 4 | 4 |
| | | PI(D30) | 24 | 23 | 95.8 | 78.9 | 99.9 | 202.5 | 116.2 | 352.8 |
| | | PII(D90) | 21 | 21 | 100 | 83.9 | 100 | 225.7 | 139.7 | 364.8 |
| | 23PPV | PRE | 23 | 2 | 8.7 | 1.1 | 28 | 5.5 | 3.5 | 8.7 |
| | | PI(D30) | 24 | 24 | 100 | 85.8 | 100 | 416.9 | 185.6 | 936.8 |
| | | PII(D90) | 23 | 21 | 91.3 | 72 | 98.9 | 180.9 | 70.3 | 465.7 |

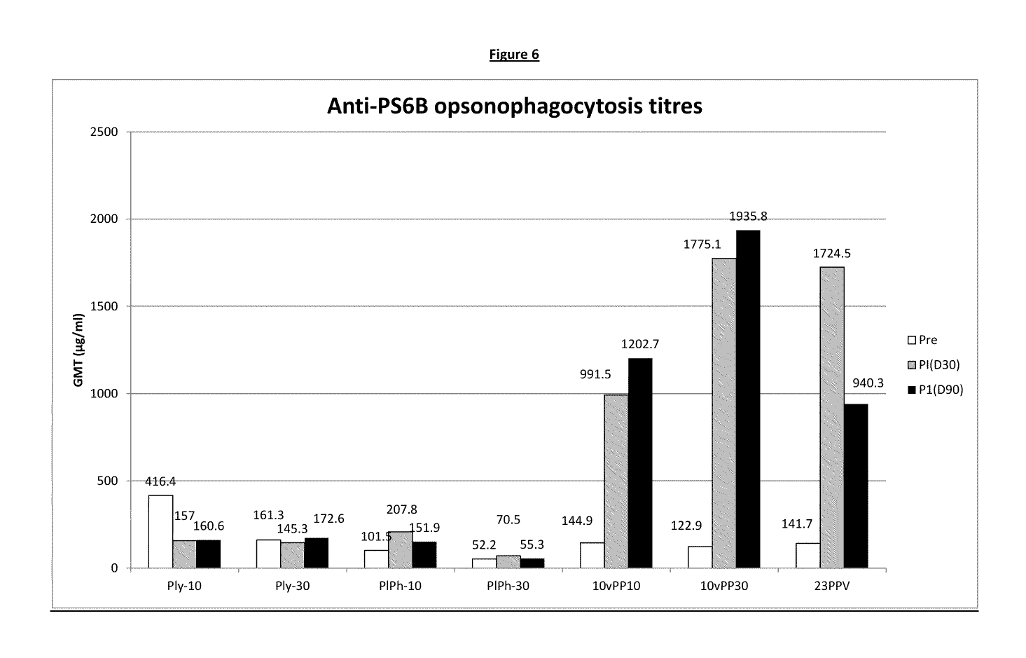

Seropositivity rates and OPA (opsonophagocytosis) GMTs for serotype 6B antibodies are described in Table 6 below, these data are illustrated in FIG. 6:

TABLE 6
Seropositivity rates and GMTs for anti-PS6B
antibodies (ATP cohort for immunogenicity)

| | | | | ≧8 | | | | GMT | | |
|---|---|---|---|---|---|---|---|---|---|---|
| | | | | | | 95% CI | | | 95% CI | |
| Antibody | Group | Timing | N | n | % | LL | UL | value | LL | UL |
| OPSONO- | Ply-10 | PRE | 8 | 7 | 87.5 | 47.3 | 99.7 | 416.4 | 75.4 | 2300.9 |
| 6B | | PI(D30) | 10 | 7 | 70 | 34.8 | 93.3 | 157 | 23.6 | 1043.7 |
| | | PII(D90) | 10 | 7 | 70 | 34.8 | 93.3 | 160.6 | 24.7 | 1043 |
| | Ply-30 | PRE | 14 | 10 | 71.4 | 41.9 | 91.6 | 161.3 | 37.7 | 689.9 |
| | | PI(D30) | 22 | 16 | 72.7 | 49.8 | 89.3 | 145.3 | 50 | 422.3 |
| | | PII(D90) | 21 | 15 | 71.4 | 47.8 | 88.7 | 172.6 | 52.4 | 568.6 |
| | PlPh-10 | PRE | 11 | 7 | 63.6 | 30.8 | 89.1 | 101.5 | 16.9 | 608 |
| | | PI(D30) | 21 | 16 | 76.2 | 52.8 | 91.8 | 207.8 | 69.8 | 618.9 |
| | | PII(D90) | 23 | 16 | 69.6 | 47.1 | 86.8 | 151.9 | 50.1 | 460.8 |
| | PlPh-30 | PRE | 10 | 5 | 50 | 18.7 | 81.3 | 52.2 | 7.3 | 375 |
| | | PI(D30) | 22 | 12 | 54.5 | 32.2 | 75.6 | 70.5 | 21 | 236.1 |
| | | PII(D90) | 23 | 11 | 47.8 | 26.8 | 69.4 | 55.3 | 16.1 | 190.3 |
| | 10vPP10 | PRE | 13 | 10 | 76.9 | 46.2 | 95 | 144.9 | 37.6 | 558.8 |
| | | PI(D30) | 24 | 24 | 100 | 85.8 | 100 | 991.5 | 583.3 | 1685.2 |
| | | PII(D90) | 24 | 24 | 100 | 85.8 | 100 | 1202.7 | 792.4 | 1825.3 |
| | 10vPP30 | PRE | 12 | 8 | 66.7 | 34.9 | 90.1 | 122.9 | 23.3 | 648.5 |
| | | PI(D30) | 24 | 24 | 100 | 85.8 | 100 | 1775.1 | 961.8 | 3276.3 |
| | | PII(D90) | 21 | 21 | 100 | 83.9 | 100 | 1935.8 | 1193.7 | 3139.2 |
| | 23PPV | PRE | 10 | 7 | 70 | 34.8 | 93.3 | 141.7 | 22.4 | 897.5 |
| | | PI(D30) | 23 | 22 | 95.7 | 78.1 | 99.9 | 1724.5 | 837.6 | 3550.7 |
| | | PII(D90) | 23 | 21 | 91.3 | 72 | 98.9 | 940.3 | 399.2 | 2214.7 |

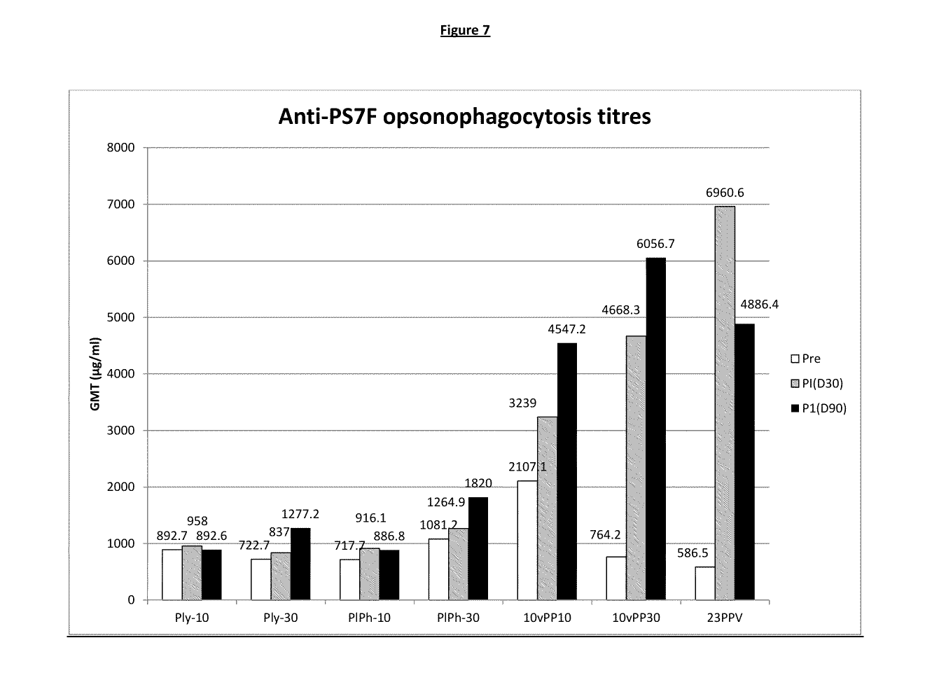

Seropositivity rates and OPA (opsonophagocytosis) GMTs for serotype 7F antibodies are described in Table 7 below, these data are illustrated in FIG. 7:

TABLE 7
Seropositivity rates and GMTs for anti-PS7Fantibodies (ATP cohort for immunogenicity)

| | | | | ≧8 | | | | GMT | | |
|---|---|---|---|---|---|---|---|---|---|---|
| | | | | | | 95% CI | | | 95% CI | |
| Antibody | Group | Timing | N | n | % | LL | UL | value | LL | UL |
| OPSONO- | Ply-10 | PRE | 8 | 8 | 100 | 63.1 | 100 | 892.7 | 407.2 | 1957.3 |
| 7F | | PI(D30) | 9 | 9 | 100 | 66.4 | 100 | 958 | 453.6 | 2023.3 |
| | | PII(D90) | 10 | 10 | 100 | 69.2 | 100 | 892.6 | 443.1 | 1798 |
| | Ply-30 | PRE | 17 | 15 | 88.2 | 63.6 | 98.5 | 722.7 | 229.1 | 2279.9 |
| | | PI(D30) | 24 | 22 | 91.7 | 73 | 99 | 837 | 380 | 1843.6 |
| | | PII(D90) | 20 | 19 | 95 | 75.1 | 99.9 | 1277.2 | 567.4 | 2875.2 |
| | PlPh-10 | PRE | 14 | 12 | 85.7 | 57.2 | 98.2 | 717.7 | 179.5 | 2869.1 |
| | | PI(D30) | 24 | 23 | 95.8 | 78.9 | 99.9 | 916.1 | 465.2 | 1804.2 |
| | | PII(D90) | 23 | 21 | 91.3 | 72 | 98.9 | 886.8 | 376.7 | 2087.3 |
| | PlPh-30 | PRE | 17 | 16 | 94.1 | 71.3 | 99.9 | 1081.2 | 468 | 2497.9 |
| | | PI(D30) | 24 | 24 | 100 | 85.8 | 100 | 1264.9 | 895.7 | 1786.3 |
| | | PII(D90) | 24 | 24 | 100 | 85.8 | 100 | 1820 | 1218.9 | 2717.5 |
| | 10vPP10 | PRE | 11 | 11 | 100 | 71.5 | 100 | 2107.1 | 964.6 | 4602.7 |
| | | PI(D30) | 24 | 24 | 100 | 85.8 | 100 | 3239 | 2209.1 | 4749.1 |
| | | PII(D90) | 24 | 24 | 100 | 85.8 | 100 | 4547.2 | 3105.4 | 6658.4 |
| | 10vPP30 | PRE | 12 | 11 | 91.7 | 61.5 | 99.8 | 764.2 | 193.3 | 3022 |
| | | PI(D30) | 24 | 24 | 100 | 85.8 | 100 | 4668.3 | 2811.9 | 7750.4 |
| | | PII(D90) | 21 | 21 | 100 | 83.9 | 100 | 6056.7 | 4211.8 | 8709.6 |
| | 23PPV | PRE | 14 | 12 | 85.7 | 57.2 | 98.2 | 586.5 | 156.8 | 2194.4 |
| | | PI(D30) | 22 | 22 | 100 | 84.6 | 100 | 6960.6 | 4531.6 | 10691.5 |
| | | PII(D90) | 23 | 23 | 100 | 85.2 | 100 | 4886.4 | 2956.8 | 8075.1 |

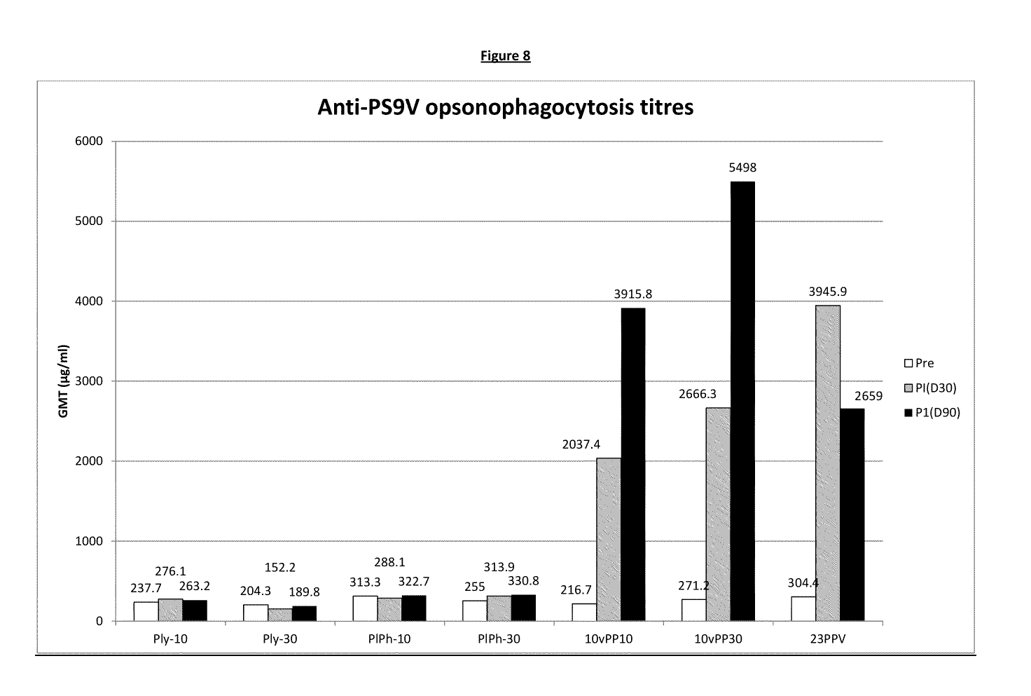

Seropositivity rates and OPA GMTs for serotype 9V antibodies are described in Table 8 below, these data are illustrated in FIG. 8:

TABLE 8
Seropositivity rates and GMTs for anti-PS9V
antibodies (ATP cohort for immunogenicity)

| | | | | ≧8 | | | | GMT | | |
|---|---|---|---|---|---|---|---|---|---|---|
| | | | | | | 95% CI | | | 95% CI | |
| Antibody | Group | Timing | N | n | % | LL | UL | value | LL | UL |
| OPSONO- | Ply-10 | PRE | 9 | 9 | 100 | 66.4 | 100 | 237.7 | 128.4 | 439.9 |
| 9V | | PI(D30) | 10 | 10 | 100 | 69.2 | 100 | 276.1 | 149.9 | 508.5 |
| | | PII(D90) | 10 | 10 | 100 | 69.2 | 100 | 263.2 | 157.7 | 439.4 |
| | Ply-30 | PRE | 19 | 17 | 89.5 | 66.9 | 98.7 | 204.3 | 87.4 | 477.8 |
| | | PI(D30) | 22 | 18 | 81.8 | 59.7 | 94.8 | 152.2 | 61.8 | 375.2 |
| | | PII(D90) | 21 | 17 | 81 | 58.1 | 94.6 | 189.8 | 70.7 | 510 |
| | PlPh-10 | PRE | 22 | 21 | 95.5 | 77.2 | 99.9 | 313.3 | 163.8 | 599.3 |
| | | PI(D30) | 24 | 23 | 95.8 | 78.9 | 99.9 | 288.1 | 156.1 | 531.5 |
| | | PII(D90) | 22 | 21 | 95.5 | 77.2 | 99.9 | 322.7 | 168.8 | 616.8 |
| | PlPh-30 | PRE | 21 | 21 | 100 | 83.9 | 100 | 255 | 161 | 403.9 |
| | | PI(D30) | 22 | 21 | 95.5 | 77.2 | 99.9 | 313.9 | 164.5 | 598.8 |
| | | PII(D90) | 23 | 22 | 95.7 | 78.1 | 99.9 | 330.8 | 173.8 | 629.6 |
| | 10vPP10 | PRE | 24 | 22 | 91.7 | 73 | 99 | 216.7 | 103.5 | 453.8 |
| | | PI(D30) | 24 | 24 | 100 | 85.8 | 100 | 2037.4 | 1208.5 | 3434.7 |
| | | PII(D90) | 24 | 24 | 100 | 85.8 | 100 | 3915.8 | 2467.4 | 6214.3 |
| | 10vPP30 | PRE | 22 | 22 | 100 | 84.6 | 100 | 271.2 | 177.8 | 413.6 |
| | | PI(D30) | 24 | 24 | 100 | 85.8 | 100 | 2666.3 | 1586.8 | 4480.2 |
| | | PII(D90) | 21 | 21 | 100 | 83.9 | 100 | 5498 | 3334.3 | 9065.8 |
| | 23PPV | PRE | 20 | 19 | 95 | 75.1 | 99.9 | 304.4 | 148.5 | 624 |
| | | PI(D30) | 24 | 24 | 100 | 85.8 | 100 | 3945.9 | 2390.3 | 6514.1 |
| | | PII(D90) | 23 | 23 | 100 | 85.2 | 100 | 2659 | 1463.6 | 4830.9 |

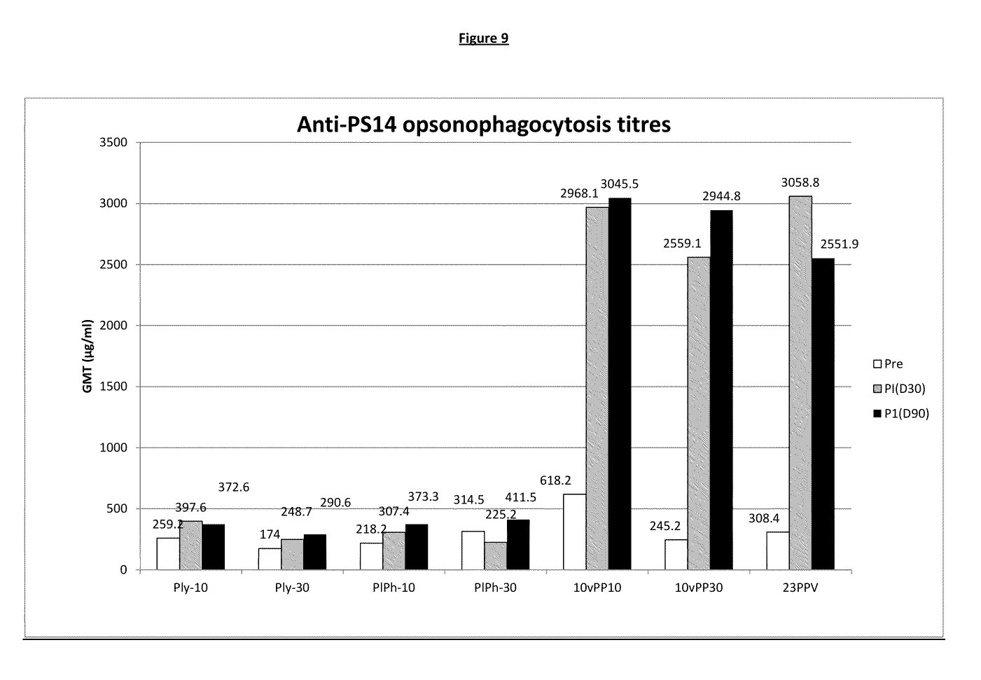

Seropositivity rates and OPA GMTs for serotype 14 antibodies are described in Table 9 below, these data are illustrated in FIG. 9:

TABLE 9
Seropositivity rates and GMTs for anti-PS14 antibodies (ATP cohort for immunogenicity)

| | | | | ≧8 | | | | GMT | | |
|---|---|---|---|---|---|---|---|---|---|---|
| | | | | | | 95% CI | | | 95% CI | |
| Antibody | Group | Timing | N | n | % | LL | UL | value | LL | UL |
| OPSONO- | Ply-10 | PRE | 9 | 8 | 88.9 | 51.8 | 99.7 | 259.2 | 67.5 | 994.5 |
| 14 | | PI(D30) | 9 | 8 | 88.9 | 51.8 | 99.7 | 397.6 | 97.5 | 1621.7 |
| | | PII(D90) | 10 | 9 | 90 | 55.5 | 99.7 | 372.6 | 110.6 | 1255.5 |
| | Ply-30 | PRE | 18 | 16 | 88.9 | 65.3 | 98.6 | 174 | 76.4 | 396.3 |
| | | PI(D30) | 22 | 20 | 90.9 | 70.8 | 98.9 | 248.7 | 122.3 | 505.7 |
| | | PII(D90) | 21 | 19 | 90.5 | 69.6 | 98.8 | 290.6 | 134.4 | 628.3 |
| | PlPh-10 | PRE | 20 | 17 | 85 | 62.1 | 96.8 | 218.2 | 85.7 | 555.5 |
| | | PI(D30) | 21 | 19 | 90.5 | 69.6 | 98.8 | 307.4 | 134.8 | 701 |
| | | PII(D90) | 21 | 19 | 90.5 | 69.6 | 98.8 | 373.3 | 167.3 | 832.9 |
| | PlPh-30 | PRE | 14 | 13 | 92.9 | 66.1 | 99.8 | 314.5 | 126.7 | 780.2 |
| | | PI(D30) | 20 | 18 | 90 | 68.3 | 98.8 | 225.2 | 98.1 | 517.2 |
| | | PII(D90) | 21 | 20 | 95.2 | 76.2 | 99.9 | 411.5 | 215.1 | 787.5 |
| | 10vPP10 | PRE | 17 | 17 | 100 | 80.5 | 100 | 618.2 | 364.9 | 1047.4 |
| | | PI(D30) | 24 | 24 | 100 | 85.8 | 100 | 2968.1 | 1713.8 | 5140.3 |
| | | PII(D90) | 24 | 24 | 100 | 85.8 | 100 | 3045.5 | 2007.2 | 4620.9 |
| | 10vPP30 | PRE | 16 | 15 | 93.8 | 69.8 | 99.8 | 245.2 | 116.4 | 516.5 |
| | | PI(D30) | 24 | 24 | 100 | 85.8 | 100 | 2559.1 | 1651 | 3966.7 |
| | | PII(D90) | 21 | 21 | 100 | 83.9 | 100 | 2944.8 | 1854 | 4677.5 |
| | 23PPV | PRE | 20 | 19 | 95 | 75.1 | 99.9 | 308.4 | 164.8 | 576.8 |
| | | PI(D30) | 24 | 24 | 100 | 85.8 | 100 | 3058.8 | 1829.4 | 5114.2 |
| | | PII(D90) | 22 | 22 | 100 | 84.6 | 100 | 2551.9 | 1518.7 | 4287.9 |

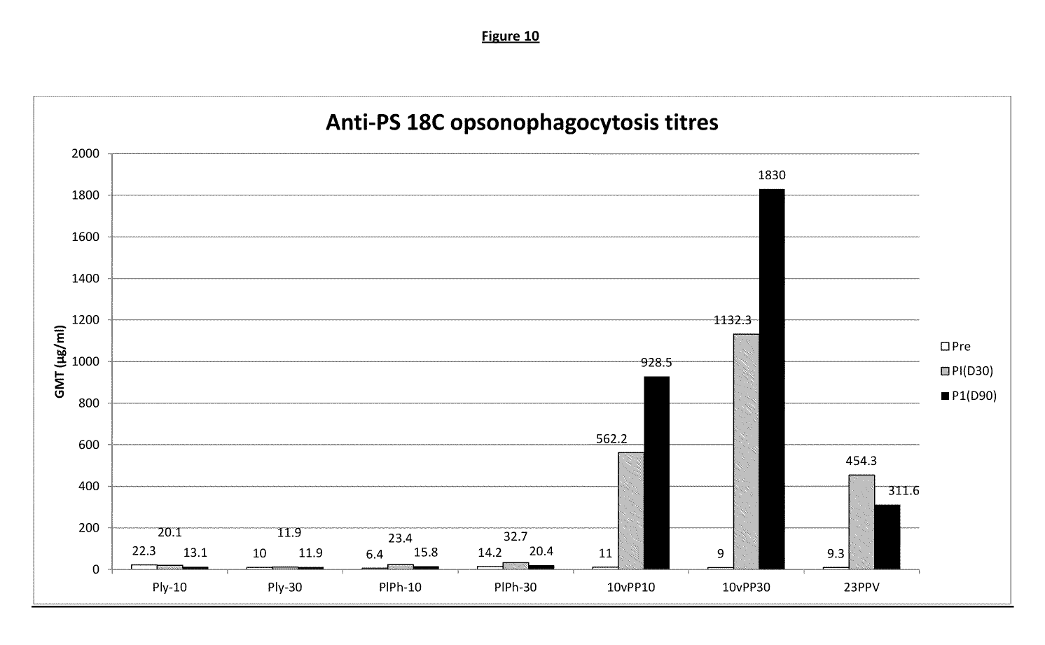

Seropositivity rates and OPA GMTs for serotype 18C antibodies are described in Table 10 below, these data are illustrated in FIG. 10:

TABLE 10
Seropositivity rates and GMTs for anti-PS18C
antibodies (ATP cohort for immunogenicity)

| | | | | ≧8 | | | | GMT | | |
|---|---|---|---|---|---|---|---|---|---|---|
| | | | | | | 95% CI | | | 95% CI | |
| Antibody | Group | Timing | N | n | % | LL | UL | value | LL | UL |
| OPSONO- | Ply-10 | PRE | 10 | 4 | 40 | 12.2 | 73.8 | 22.3 | 4.5 | 111.2 |
| 18C | | PI(D30) | 9 | 3 | 33.3 | 7.5 | 70.1 | 20.1 | 3.1 | 130.7 |
| | | PII(D90) | 10 | 3 | 30 | 6.7 | 65.2 | 13.1 | 3.2 | 53.8 |
| | Ply-30 | PRE | 18 | 4 | 22.2 | 6.4 | 47.6 | 10 | 3.9 | 25.7 |
| | | PI(D30) | 21 | 7 | 33.3 | 14.6 | 57 | 11.9 | 5.2 | 27.2 |
| | | PII(D90) | 17 | 6 | 35.3 | 14.2 | 61.7 | 11.9 | 4.8 | 29.6 |
| | PlPh-10 | PRE | 15 | 3 | 20 | 4.3 | 48.1 | 6.4 | 3.3 | 12.7 |
| | | PI(D30) | 20 | 9 | 45 | 23.1 | 68.5 | 23.4 | 8.6 | 64 |
| | | PII(D90) | 22 | 8 | 36.4 | 17.2 | 59.3 | 15.8 | 6.3 | 39.9 |
| | PlPh-30 | PRE | 15 | 7 | 46.7 | 21.3 | 73.4 | 14.2 | 5.9 | 34.2 |
| | | PI(D30) | 24 | 14 | 58.3 | 36.6 | 77.9 | 32.7 | 14.2 | 75.4 |
| | | PII(D90) | 20 | 9 | 45 | 23.1 | 68.5 | 20.4 | 7.9 | 52.7 |
| | 10vPP10 | PRE | 18 | 6 | 33.3 | 13.3 | 59 | 11 | 5.2 | 23.3 |
| | | PI(D30) | 24 | 23 | 95.8 | 78.9 | 99.9 | 562.2 | 286 | 1105 |
| | | PII(D90) | 24 | 24 | 100 | 85.8 | 100 | 928.5 | 613.2 | 1405.8 |
| | 10vPP30 | PRE | 20 | 4 | 20 | 5.7 | 43.7 | 9 | 4.1 | 19.6 |
| | | PI(D30) | 24 | 24 | 100 | 85.8 | 100 | 1132.3 | 750.8 | 1707.6 |
| | | PII(D90) | 21 | 21 | 100 | 83.9 | 100 | 1830 | 1220.6 | 2743.7 |
| | 23PPV | PRE | 18 | 4 | 22.2 | 6.4 | 47.6 | 9.3 | 4.1 | 20.8 |
| | | PI(D30) | 24 | 23 | 95.8 | 78.9 | 99.9 | 454.3 | 217.1 | 950.7 |
| | | PII(D90) | 22 | 20 | 90.9 | 70.8 | 98.9 | 311.6 | 134.9 | 719.5 |

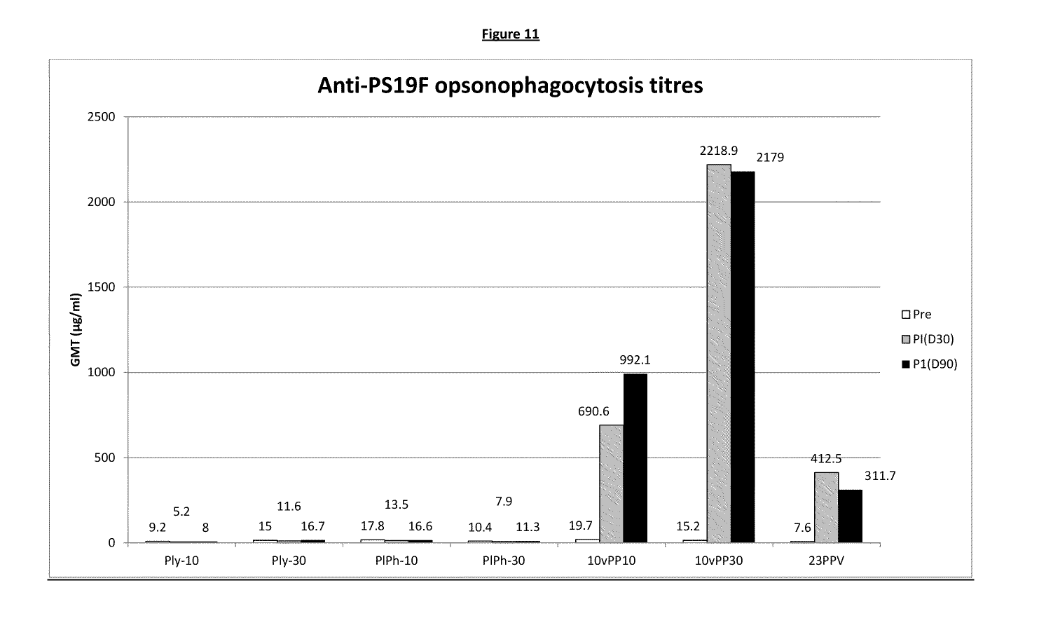

Seropositivity rates and OPA GMTs for serotype 19F antibodies are described in Table 11 below, these data are illustrated in FIG. 11:

TABLE 11
Seropositivity rates and GMTs for anti-PS19F
antibodies (ATP cohort for immunogenicity)

| | | | | ≧8 | | | | GMT | | |
|---|---|---|---|---|---|---|---|---|---|---|
| | | | | | | 95% CI | | | 95% CI | |
| Antibody | Group | Timing | N | n | % | LL | UL | value | LL | UL |
| OPSONO- | Ply-10 | PRE | 10 | 4 | 40 | 12.2 | 73.8 | 9.2 | 4 | 21.4 |
| 19F | | PI(D30) | 8 | 1 | 12.5 | 0.3 | 52.7 | 5.2 | 2.8 | 9.8 |
| | | PII(D90) | 10 | 5 | 50 | 18.7 | 81.3 | 8 | 4.7 | 13.7 |
| | Ply-30 | PRE | 20 | 10 | 50 | 27.2 | 72.8 | 15 | 7.2 | 31.4 |
| | | PI(D30) | 23 | 10 | 43.5 | 23.2 | 65.5 | 11.6 | 6.1 | 21.9 |
| | | PII(D90) | 21 | 11 | 52.4 | 29.8 | 74.3 | 16.7 | 7.9 | 35.5 |
| | PlPh-10 | PRE | 19 | 13 | 68.4 | 43.4 | 87.4 | 17.8 | 9.8 | 32.3 |
| | | PI(D30) | 23 | 12 | 52.2 | 30.6 | 73.2 | 13.5 | 7 | 25.7 |
| | | PII(D90) | 23 | 14 | 60.9 | 38.5 | 80.3 | 16.6 | 8.4 | 32.8 |
| | PlPh-30 | PRE | 21 | 8 | 38.1 | 18.1 | 61.6 | 10.4 | 5.7 | 19.2 |
| | | PI(D30) | 23 | 8 | 34.8 | 16.4 | 57.3 | 7.9 | 4.9 | 12.6 |
| | | PII(D90) | 24 | 10 | 41.7 | 22.1 | 63.4 | 11.3 | 5.9 | 21.7 |
| | 10vPP10 | PRE | 21 | 10 | 47.6 | 25.7 | 70.2 | 19.7 | 7.8 | 49.8 |
| | | PI(D30) | 21 | 21 | 100 | 83.9 | 100 | 690.6 | 374.4 | 1273.7 |
| | | PII(D90) | 24 | 24 | 100 | 85.8 | 100 | 992.1 | 532.9 | 1847.2 |
| | 10vPP30 | PRE | 20 | 11 | 55 | 31.5 | 76.9 | 15.2 | 7.7 | 30.1 |
| | | PI(D30) | 23 | 23 | 100 | 85.2 | 100 | 2218.9 | 1351.2 | 3643.6 |
| | | PII(D90) | 21 | 21 | 100 | 83.9 | 100 | 2179 | 1404.4 | 3380.9 |
| | 23PPV | PRE | 20 | 7 | 35 | 15.4 | 59.2 | 7.6 | 4.9 | 11.7 |
| | | PI(D30) | 20 | 20 | 100 | 83.2 | 100 | 412.5 | 189.4 | 898.4 |
| | | PII(D90) | 23 | 22 | 95.7 | 78.1 | 99.9 | 311.7 | 141.7 | 685.6 |

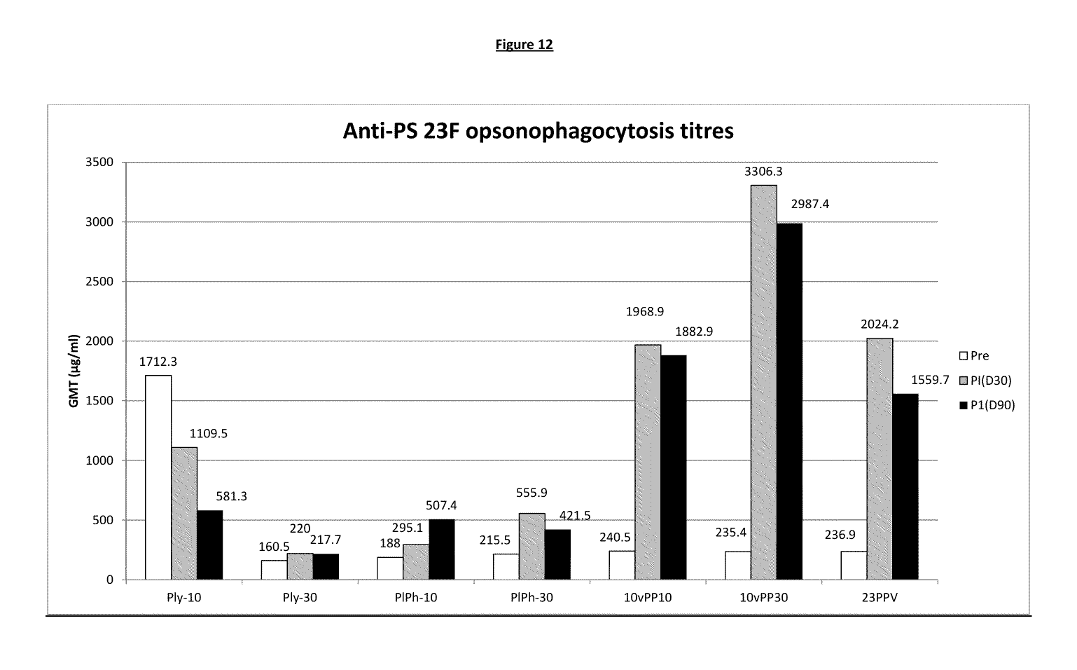

Seropositivity rates and OPA GMTs for serotype 23F antibodies are described in Table 12 below, these data are illustrated in FIG. 12:

TABLE 12
Seropositivity rates and GMTs for anti-PS23F
antibodies (ATP cohort for immunogenicity)

| | | | | ≧8 | | | | GMT | | |
|---|---|---|---|---|---|---|---|---|---|---|
| | | | | | | 95% CI | | | 95% CI | |
| Antibody | Group | Timing | N | n | % | LL | UL | value | LL | UL |
| OPSONO- | Ply-10 | PRE | 6 | 6 | 100 | 54.1 | 100 | 1712.3 | 705.1 | 4158.6 |
| 23F | | PI(D30) | 9 | 9 | 100 | 66.4 | 100 | 1109.5 | 425.5 | 2893 |
| | | PII(D90) | 10 | 9 | 90 | 55.5 | 99.7 | 581.3 | 126.3 | 2674.8 |
| | Ply-30 | PRE | 16 | 11 | 68.8 | 41.3 | 89 | 160.5 | 40 | 644.8 |
| | | PI(D30) | 19 | 14 | 73.7 | 48.8 | 90.9 | 220 | 66 | 733.8 |
| | | PII(D90) | 18 | 14 | 77.8 | 52.4 | 93.6 | 217.7 | 70.9 | 668.3 |
| | PlPh-10 | PRE | 12 | 8 | 66.7 | 34.9 | 90.1 | 188 | 29.2 | 1211 |
| | | PI(D30) | 21 | 16 | 76.2 | 52.8 | 91.8 | 295.1 | 92.9 | 937.5 |
| | | PII(D90) | 19 | 16 | 84.2 | 60.4 | 96.6 | 507.4 | 170.4 | 1510.5 |
| | PlPh-30 | PRE | 9 | 6 | 66.7 | 29.9 | 92.5 | 215.5 | 17.6 | 2635.2 |
| | | PI(D30) | 21 | 19 | 90.5 | 69.6 | 98.8 | 555.9 | 230.5 | 1340.8 |
| | | PII(D90) | 22 | 18 | 81.8 | 59.7 | 94.8 | 421.5 | 140.9 | 1261 |
| | 10vPP10 | PRE | 19 | 13 | 68.4 | 43.4 | 87.4 | 240.5 | 58.6 | 987.6 |
| | | PI(D30) | 24 | 24 | 100 | 85.8 | 100 | 1968.9 | 1256.5 | 3085.1 |
| | | PII(D90) | 24 | 24 | 100 | 85.8 | 100 | 1882.9 | 1176.2 | 3014.3 |
| | 10vPP30 | PRE | 17 | 12 | 70.6 | 44 | 89.7 | 235.4 | 55.6 | 996.7 |
| | | PI(D30) | 24 | 24 | 100 | 85.8 | 100 | 3306.3 | 2280.7 | 4793.1 |
| | | PII(D90) | 21 | 21 | 100 | 83.9 | 100 | 2987.4 | 1977.1 | 4513.9 |
| | 23PPV | PRE | 15 | 11 | 73.3 | 44.9 | 92.2 | 236.9 | 56.8 | 988.4 |
| | | PI(D30) | 24 | 24 | 100 | 85.8 | 100 | 2024.2 | 1237.8 | 3310.1 |
| | | PII(D90) | 23 | 22 | 95.7 | 78.1 | 99.9 | 1559.7 | 778.7 | 3123.8 |

Summary of Results

All candidate vaccine formulations were safe and well tolerated. No clinically significant changes in the haematology, biochemistry and urine parameters were observed over time.

During the 7-day post-vaccination period after dose 1, maximum 4.2% to 12.5% of doses of the candidate vaccine formulations were followed by grade 3 solicited local and general AEs, respectively. These incidences were within the same ranges as those observed in the 23PPV comparator group (4.2% of doses). No grade 3 fever (oral temperature >39.5° C.) was reported.

No trend towards higher incidences of grade 3 solicited local and/or general AEs was generally observed in all groups with consecutive dose of candidate vaccine formulations.

Grade 3 unsolicited AEs were reported following 4.5% to 13.3% of doses for the candidate vaccine formulations. Unsolicited AEs considered by the investigator to be causally related to vaccination were reported following 10.4% to 33.3% of doses for the candidate vaccine formulations and were mainly local reactions.

No SAEs were reported in the subjects receiving the candidate vaccine formulations.

The formulations containing the pneumococcal protein dPly with or without PhtD were immunogenic. Increases in the anti-Ply antibody GMCs were observed following each vaccination in the groups vaccinated with dPly containing formulations; increase in anti-PhtD antibody GMCs were observed following each vaccination in groups vaccinated with PhtD containing formulations. The highest anti-Ply and anti-PhtD antibody GMCs were observed for the formulations containing 30 μg of dPly and PhtD proteins (i.e in the group PIPh-30 followed by 10vPP30 group).

Free protein formulations (alum adsorbed) do not induce an OPA responses One month post-dose 1 and one month post-dose 2, for each of the vaccine pneumococcal serotypes, at least 95.7% of subjects receiving the formulation combining pneumococcal proteins (dPly and PhtD) and 10Pn-PD-DiT had OPA titres≧8. An increase in OPA GMTs was observed from post-dose 1 to post-dose 2 timepoint for some of the pneumococcal serotypes. For a majority of the tested serotypes (6B, 7F, 9V, 18C, 19F, 23F) the calculated OPA GMT point estimates post dose 1 or post dose 2 were higher in the 10vPP30 groups as compared to the 10vPP10 group.

### Example 3

### Clinical Trial to Study Efficacy of Vaccines Comprising High and Low Doses of dPly and PhtD Carried Out in Infants

A phase II, randomized, controlled, observer-blind study to assess the safety, reactogenicity and immunogenicity of two formulations of GlaxoSmithKline (GSK) Biologicals' Streptococcus pneumoniae protein containing vaccine given as a 3-dose primary vaccination course co-administered with DTPa-HBV-IPV/Hib vaccine during the first 6 months (Epoch 1) of life and as a booster dose at 12-15 months of age (Epoch 2).

This clinical trial was carried out using 4 parallel groups;
1. 10PCV/dPly/PhtD-10-AIPO₄ group (‘10vPP10’ in result tables and figures): subjects who received GSK Biologicals' 10PCV/dPly/PhtD-10-AIPO₄ vaccine co-administered with DTPa-HBV-IPV/Hib vaccine.
2. 10PCV/dPly/PhtD-30-AIPO₄group (‘10vPP30’ in result tables and figures): subjects who received GSK Biologicals' 10PCV/dPly/PhtD-30-AIPO₄ vaccine co-administered with DTPa-HBV-IPV/Hib vaccine.
3. 10PCV group (‘10Pn’ in result tables and figures): subjects who received GSK Biologicals' 10PCV vaccine co-administered with DTPa-HBV-IPV/Hib vaccine.
4. Prev13 group (‘13Pn-DTPa’ in result tables and figures): subjects who received Pfizer's commercially available 13-valent pneumococcal conjugate vaccine (Prevenar 13) co-administered with DTPa-HBV-IPV/Hib vaccine.

The vaccines were given to healthy infants according to a 3-dose primary vaccination schedule at 2, 3, 4 months of age (Epoch 1, completed) to be followed by a booster dose at 12-15 months of age (Epoch 2, study ongoing).

Safety/Reactogenicity Methods

Within Group Assessment (Descriptive Analysis):
The percentage of subjects reporting each individual solicited local and general AE (adverse event) during the 7-day (Day 0-Day 6) solicited follow-up period were tabulated for each group, after each vaccine dose and overall primary doses, with exact 95% Cl (confidance interval). The percentage of doses followed by each individual solicited local and general AE was tabulated for each group, over the full primary vaccination course, with exact 95% Cl. The same tabulations were performed for grade 3 solicited AEs and for solicited AEs with causal relationship to vaccination. For redness and swelling, grade 2 or 3 AEs were also tabulated. Occurrence of fever was reported per 0.5° C. cumulative increments. All the above tabulations for each individual solicited adverse event were also performed for the 4-day follow-up period after each vaccination (Day 0-Day 3).
The proportion of subjects/doses with at least one report of unsolicited AE classified by the Medical Dictionary for Regulatory Activities (MedDRA) and reported up to 30 days after primary vaccination was tabulated with exact 95% Cl for each group. The same tabulation was performed for grade 3 unsolicited AEs and for unsolicited AEs with a relationship to vaccination.
The proportion of AEs that resulted in a medically attended visit was also tabulated.
SAEs (serious adverse events) and withdrawal(s) due to SAE(s) were described in detail.

Laboratory Assays and Time Points for Immunogenicity Evaluation

Blood samples were collected at Visit 1 and Visit 4 during the Epoch 001. After blood centrifugation and serum separation, samples were stored at −20° C. until collection by the sponsor. The aliquots of serum (approximately 2.5 mL) were sent to GSK Biologicals for the serological tests described in the following paragraphs.
Pneumococcal serotype specific total IgG antibodies (antibodies to 1, 3, 4, 5, 6A, 6B, 7F, 9V, 14, 18C, 19A, 19F and 23F) were each measured by 22F-inhibition ELISA (enzyme-linked immunosorbent assay) as described for example 2. The antibody concentration was determined by logistic log comparison of the ELISA curves with a standard reference serum 89-SF available from the US Food and Drug Administration (FDA) for which concentration of IgG and IgM to the 10 serotypes are known in μg/mL. The cut-off of the assay was 0.05 μg/mL.
Opsonophagocytic activity (OPA) for antibodies against each of the pneumococcal serotypes 1, 3, 4, 5, 6A, 6B, 7F, 9V, 14, 18C, 19A, 19F and 23F was measured by an OPA assay (similar to that described in example 2). The results were presented as the dilution of serum (opsonic titre) able to sustain 50% killing of live pneumococci under the assay conditions. The cut-off of the assay was an opsonic titre of 8.
Anti-Ply and anti-PhtD antibodies were quantified using individual ELISA. Concentration of specific anti-Ply and anti-PhtD antibodies was determined using a standard reference serum. The cut-off of the assay was 100 EL.U/mL for PD, 12 EL.U/mL for dPly and 17 EL.U/mL for PhtD.

Safety and Reactogenicity Results (Primary Outcome of the Study)

The safety analysis was performed on the Total vaccinated cohort. Solicited adverse events During the 7-day post-vaccination period:
Redness was the most frequently reported solicited local AE in each group (after 43.6%, 44.7%, 40.1% and 41.1% of doses in the 10PCV/dPly/PhtD-10-AIPO₄, 10PCV/dPly/PhtD-30-AIPO₄, 10PCV and Prev13 groups, respectively). No more than 3.3% of doses were followed by grade 3 solicited local AEs of given category, in each group.
Irritability was the most frequently reported solicited general AE in each group (after 55.5%, 55.5%, 55.0% and 56.6% of doses in the 10PCV/dPly/PhtD-10-AIPO₄, 10PCV/dPly/PhtD-30-AIPO₄, 10PCV and Prev13 groups, respectively). No more than 4.8% of doses were followed by grade 3 general solicited AEs of given category, in each group and most of them were considered by the investigator to be causally related to vaccination.
No increase in the incidence of solicited local and general AEs was observed with consecutive doses of either 10PCV/dPly/PhtD-10-AIPO₄ or 10PCV/dPly/PhtD-30-AIPO₄,vaccines, during the 3-dose primary vaccination course.

Unsolicited Adverse Events

During the 31-day post-primary vaccination period:
16.9%, 21.1%, 20.1% and 19.7% of doses in the 10PCV/dPly/PhtD-10-AIPO₄, 10PCV/dPly/PhtD-30-AIPO₄, 10PCV and Prev13 group, respectively were followed by at least one unsolicited AE.
Grade 3 unsolicited AEs were reported after 0.0%, 0.2%, 0.5% and 0.2% of doses in the 10PCV/dPly/PhtD-10-AIPO₄, 10PCV/dPly/PhtD-30-AIPO₄, 10PCV and Prev13 group, respectively. Of these, one grade 3 AE (hypotonic hyporesponsive episode) reported in the 10PCV group was considered by the investigator to be causally related to vaccination.
Unsolicited AEs considered by the investigator to be causally related to vaccination were reported for 0.2%, 0.5%, 0.9% and 0.0% of doses in the 10PCV/dPly/PhtD-10-AIPO₄, 10PCV/dPly/PhtD-30-AIPO₄, 10PCV and Prev13 group, respectively.

Serious Adverse Events:

During the primary epoch (from dose 1 up to visit 4), at least one SAE was reported for 20 subjects: 5 out of 146 (3.4%) vaccinated subjects in the 10PCV/dPly/PhtD-10-AIPO₄ group, 2 out of 142 (1.4%) vaccinated subjects in the 10PCV/dPly/PhtD-30-AIPO₄group, 10 out of 145 (6.9%) vaccinated subjects in the 10PCV group and 3 out of 142 (2.1%) vaccinated subjects in the Prev13 group. Of these, one SAE (hypotonic hyporesponsive episode) reported as the grade 3 AE during the 31-day follow-up period in the 10Pn group was considered by the investigator to be causally related to vaccination. All SAEs resolved without sequelae, except one SAE (psychomotor retardation) in the 10Pn group which was not resolved at the time of visit

4. No Fatal SAEs were Reported.

Withdrawals Due to Adverse Events/Serious Adverse Events:

One subject in the 10PCV group (subject number 607) was withdrawn from the study due to a SAE (hypotonic-hyporesponsive episode) which was considered by the investigator to be causally related to vaccination. This event resolved without sequelae after 6 days.

### Example 4

### Formulation of Multivalent Vaccines Comprising a PE-PilA Fusion Protein

3 vaccines were designed:

10V: A ten valent (10V) vaccine containing the following ten S. pneumoniae capsular saccharide conjugates: capsular saccharide from serotype 1 conjugated to protein D (1-PD), capsular saccharide from serotype 4 conjugated to protein D (4-PD), capsular saccharide from serotype 5 conjugated to protein D (5-PD), capsular saccharide from serotype 6B conjugated to protein D (6B-PD), capsular saccharide from serotype 7F conjugated to protein D (7F-PD), capsular saccharide from serotype 9V conjugated to protein D (9V-PD), capsular saccharide from serotype 14 conjugated to protein D (14-PD), capsular saccharide from serotype 23F conjugated to protein D (23F-PD), capsular saccharide from serotype 18C conjugated to tetanus toxoid (18C-TT) and capsular saccharide from serotype 19F conjugated to Diphtheria Toxin (19F-DT).
12V: A twelve valent (12V) vaccine containing the same ten S. pneumoniae capsular saccharide conjugates as 10V with an additional two S. pneumoniae saccharide conjugates, 19A conjugated to CRM197 (19ACRM) and 6A conjugated to CRM197 (6ACRM).
12V+proteins (12V+prot): A vaccine containing the same twelve S. pneumoniae capsular saccharide conjugates as 12V with the addition of PhtD, dPly and PE-PilA fusion protein.

Preparation of the Antigens

The antigens were prepared as described in example 1. The PEPilA antigen was prepared as described in WO2012/139225.

Formulation of the Vaccines

The 10V vaccine contains S. pneumoniae capsular saccharide serotype 1, 4, 5, 6B, 7F, 9V, 14, 18C, 19F and 23F conjugates adsorbed onto aluminium phosphate together at a human dose of 1, 3, 1, 1, 1, 1, 1, 3, 3, 1 μg (the saccharides were individually adsorbed to aluminium phosphate, they were then mixed together and the level of aluminium phosphate adjusted to 500 μg).

The 12V vaccine was made in the same way as the 10V vaccine with additional serotypes 19A and 6A conjugates at doses of 2 μg adsorbed onto aluminium phosphate added.

The 12V+proteins vaccine was made in the same way as the 12V vaccine except proteins PhtD, dPly and PE-PilA were added. The 12 conjugates and the proteins were mixed together using the dosages described for 12V above and the proteins at dosages of 30 μg each (note that this refers to 30 μg of PE-PilA, not 30 μg of PE and 30 μg of PilA).

### Example 5

### Comparison of the Immunogenicity of 10V, 12V and 12V+Proteins Vaccines in Mice

Description of the Anti-Pneumococcal Polysaccharide PS (Polysaccharide) ELISA

Microplates were coated for 2 hours at 37° C. with capsular polysaccharide (CPS) (100 μl per well of 2.5 μg/ml of PS1 and PS3, 5 μg/ml of PS4, 5, 6A, 6B, 7F, 9V or 14; 10 μg/ml of PS19A and 23F or 40 μg/ml of PS18C and PS19F in PBS). The plates were washed three times with NaCl 150 mM (0.9%)-Polysorbate 20 0.05%. Sera was diluted (½ for 6A and 6B and 1/10 for the other serotypes) in PBS-Polysorbate20 0.05% containing CPS (1 mg CPS/ml of non diluted serum except or 6A and 6B which was at 2.5 mg/ml) V/V and incubated for 1 hour at 37° C. in order to neutralize antibodies directed to the CPS. The sera from mice immunised as described in the section entitled ‘immunogenicity of three vaccine formulations in mice’ or a reference serum (an internal reference calibrated with Chrompure mouse IgG) was added to the microwells and serially diluted 100 μl (two-fold dilution step) in PBS-Polysorbate20 0.05%. The plates were incubated under agitation for 30 minutes at room temperature. The plates were washed as above and anti-mouse IgG antibodies conjugated to peroxidase (100 μl per well) was added, the plates were incubated for 30 minutes at room temperature with shaking. After washing, the substrate (4 mg of OPDA (ortho phenylen-diamine) in 10 ml of citrate 0.1M pH 4.5-4.6 and 5 μl of H₂O₂) was added to each well (100 μl) and the plates incubated for 15 minutes in the dark. The reaction was stopped by addition of HCl 1N (50 μl). The absorbance was read at 490 nm or 620 nm for the reference using a spectrophotometer. The color developed is directly proportional to the amount of antibody present in the serum.

Description of the ELISA to measure PD, PE and PilA Antibodies

Plates were coated overnight at 4° C. with 100 μl per well of 2 μg/ml of PD (1 mg/ml), 2 μg/ml of PE (1500 μg/ml), 2 μg/ml of PilA (3660 μg/ml) in carbonate buffer pH 9.6. The plates were washed four times with NaCl 0.9% Polysorbate 20 0.05%. For PE and PilA ELISA, the plates were saturated for 30 min at room temperature (with shaking) with PBS-BSA1%. After washing, sera from mice immunised as described in the section entitled ‘immunogenicity of three vaccine formulations in mice’ or a reference serum (an internal reference calibrated with Chrompure mouse IgG) was added to microwells and serially diluted 100 μl (two-fold dilution step) in PBS Polysorbate 20 0.05% (for the PD assay) and PBS Polysorbate 20 0.05% BSA 0.1% (for the PE and PilA assay). The plates were then incubated for 30 min at room temperature with agitation. After washing, the plates were incubated with an anti-mouse IgG antibody conjugated to peroxidase (100 μl per well) at room temperature for 30 minutes with shaking. The plates were then washed as above and the substrate conjugate (4 mg of OPDA (ortho phenylen-diamine) in 10 ml of citrate 0.1M pH 4.5-4.6 and 5 μl of H₂O₂) as added to each well (100 μl) for 15 min in darkness. The reaction was stopped by addition of HCl 1N 50 μl and the absorbance was read at 490 nm (620 nm for the reference).

Description of the ELISA to Measure PhtD and dPly Antibodies

Plates were coated for 2 hours at 37° C. with 100 μl per well of 1 μg/ml of PhtD (1021 μg/ml) or 4 μg/ml of Ply (367 μg/ml). The plates were then washed three times with NaCl 0.09% Polysorbate 0.05%. After washing, sera from mice immunised as described in the section entitled ‘immunogenicity of three vaccine formulations in mice’ or a reference (an internal reference calibrated with Chrompure mouse IgG) (was added to microwells and serially diluted 100 μl (two-fold dilution step) in PBS Polysorbate 20 0.05%. The plates were incubated for 30 min at room temperature with agitation. After washing, the plates were incubated with an anti-mouse IgG antibody conjugated to peroxidase (100 μl per well) at room temperature for 30 minutes with shaking. The plates were washed as above and the substrate conjugate (4 mg of OPDA in 10 ml of citrate 0.1M ph 4.5 and 5 μl of H₂O₂) was added to each well (100 μl) for 15 min in darkness. The reaction was stopped by addition of HCl 1N 50 μl and the absorbance was read at 490 nm (620 nm for the reference filter).

Description of Opsonophagocytosis Assay (OPA)

Serum samples were heated for 45 min at 56° C. to inactivate any remaining endogenous complement. Twenty-five microlitres aliquots of each 1:2 diluted serum sample were two-fold serially diluted in 25 μl OPA buffer (HBSS (Hank's Balanced Salt Solution)-14.4% inactivated FCS (Foetal Calf Serum)) per well of a 96-well round bottom microtitre plate. Subsequently, 25 μl of a mixture of activated HL-60 cells (1×107 cells/ml), freshly thawed pneumococcal working seed and freshly thawed baby rabbit complement in an e.g. 4/2/1 ratio (v/v/v) (except for serotypes 1, 6B and 6A for which the ratio was 4/2/2) were added to the diluted sera to yield a final volume of 50 μl. The assay plate was incubated for 2 h at 37° C. with orbital shaking (210 rpm) to promote the phagocytic process. The reaction was stopped by laying the microplate on ice for at least 1 min, the plate is kept on ice until use. A 20 μl aliquot of each well of the plate was then transferred into the corresponding well of a 96-well flat bottom microplate and 50 μl of Todd-Hewitt Broth-0.9% agar was added to each well. After overnight incubation at 37° C. and 5% CO₂, pneumococcal colonies which appeared in the agar were counted using an automated image analysis system (KS 400, Zeiss, Oberkochen, Germany). Eight wells without serum sample were used as bacterial controls to determine the number of pneumococci per well. The mean number of CFU of the control wells was determined and used for the calculation of the killing activity for each serum sample. The OPA titre for the serum samples was determined by the reciprocal dilution of serum able to facilitate 50% killing of the pneumococci. The opsonophagocytic titre was calculated by using a 4-parameter curve fit analysis.

Immunogenicity of Three Vaccine Formulations in Mice.

2 groups of 27 female Balb/c mice were immunized by intramuscular (IM) injections on days 0, 14 and 28 with 1/10 human dose of different formulation including Proteins alone (PhtD, dPly and PEPilA—results not presented), Prevnar 13 (™ a commercially available Streptococcal vaccine—results not presented) 10V, 12V (DSP2A017) and 12V+proteins (DSP2A012) GMP lots. Mice received in a different leg (mimicking co-administration at different sites in infants in clinical trials) 1/10^{th} human dose of Infanrix Hexa (™ a vaccine comprising diphtheria toxoid, tetanus toxoid, pertussis toxoid, filamentous haemmagglutinin, pertactin, hepatitis B surface antigen, inactivated polio virus types 1, 2 and 3 and Haemophilus influenzae b saccharide (PRP)).

Anti-IgG levels and opsonophagocytosis titers were determined respectively in individual and pooled sera collected at day 42.

The potential of the 12V+proteins vaccine to induce IgG antibody titers and opsonic activity was evaluated and compared to that of the 12V and 10V vaccines.

Sera from mice injected with the different formulations were tested in ELISA (as described above) against the polysaccharide serotypes and proteins and in OPA against the 12 polysaccharide serotypes.

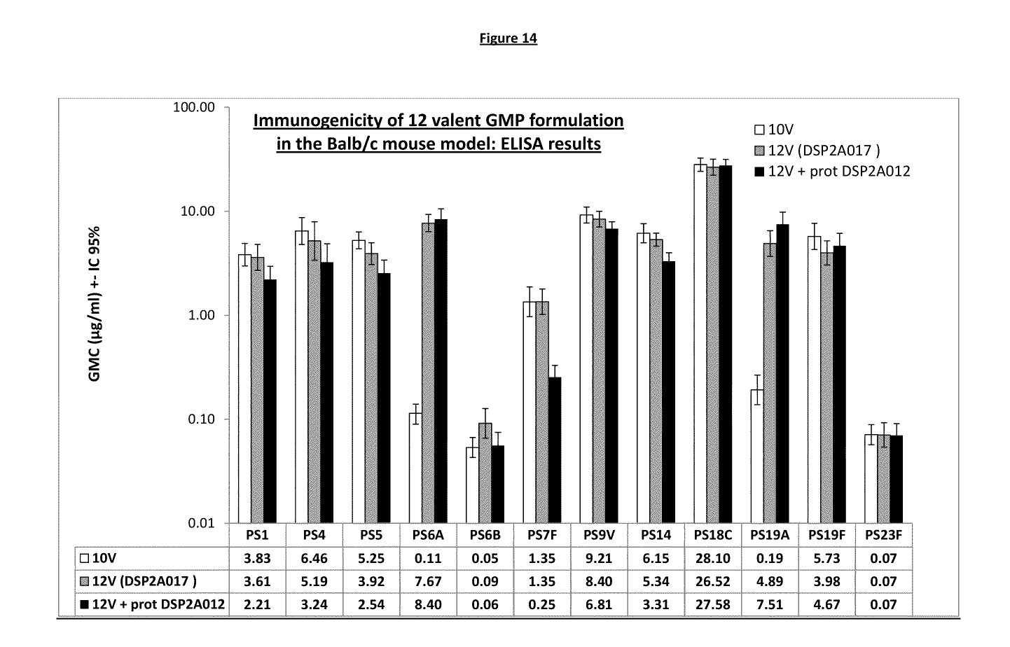

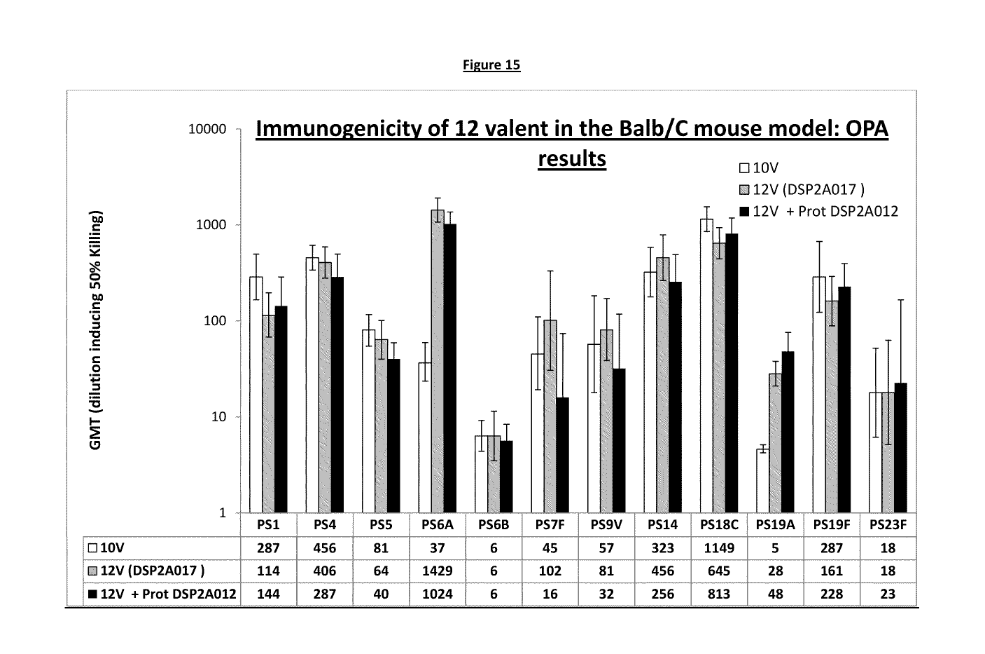

The 12V+proteins vaccine induced similar response to the 12V for most serotypes (FIGS. 14 and 15).

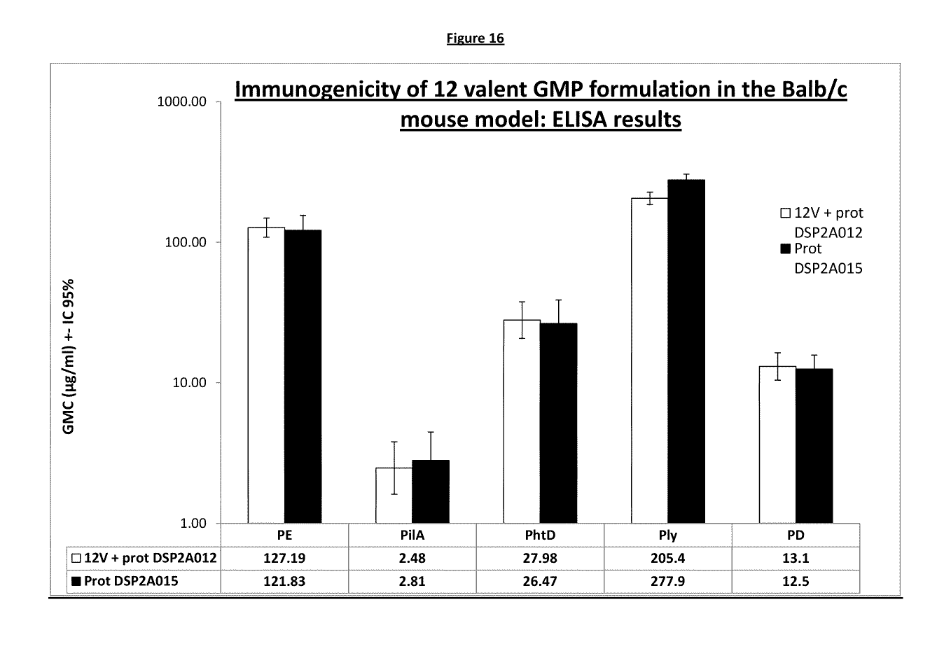

The results in FIG. 16 demonstrated that there was no statistical difference between the PE, PilA, PhtD, Ply and PD antibody responses measured for the 12V+formulation and a formulation that did not comprises the S. pneumoniae saccharide conjugates.

### Example 6

### Comparison of the Immunogenicity of 10V, 12V and 12V+Protein Vaccines in Guinea Pigs

Description of ELISA Anti-Pneumococcal Polysaccharide PS

Microplates were coated for 2 hours at 37° C. with 100 μl per well of 2.5 μg/ml of PS1, 5 μg/ml of PS4, 5, 6A, 6B, 7F, 9V or 14; 10 μg/ml of PS19A and 23F, 40 μg/ml of PS18C and PS19F or Affinipure Goat anti-guinea pig IgG (2.4 mg/ml) diluted to 2 μg/ml for the reference wells in PBS. The plates were washed three times with NaCl 150 mM (0.9%)-Polysorbate20 0.05%. Pooled serum from each group was diluted (½ for P δ 6A and 6B and 1/10 for all the other serotypes) in PBS-Polysorbate20 0.05% containing CPS (1 mg CPS/ml of non diluted serum except for 6A and 6B at 2.5 mg/ml) V/V and incubated for 1 hour at 37° C. in order to neutralize antibodies directed to CPS. The serum from guinea pigs immunised as described in the section entitled ‘immunogenicity of three vaccine formulations in guinea pigs’ was added to the microwells and serially diluted 100 μl (two-fold dilution step) in PBS-Polysorbate20 0.05% or a reference serum (Chrompure guinea pig IgG (11 mg/ml) diluted to 0.25 μg/ml in PBS-polysorbate20 0.05%) was added. The plates were incubated under agitation for 30 minutes at room temperature. The plates were washed as above and anti-guinea pig IgG antibodies conjugated to peroxidase (100 μl per well) were added and the plates incubated for 30 minutes at room temperature with shaking. After washing, the substrate (4 mg of OPDA in 10 ml of citrate 0.1M pH 4.5-4.6 and 5 μl of H₂O₂) was added to each well (100 μl) and incubated for 15 minutes in the dark. The reaction was stopped by addition of HCl 1N. Absorbance was read at 490 nm (620 nm for the reference) using a spectrophotometer. The color developed is directly proportional to the amount of antibody present in the serum.

Description of the ELISA to Measure Anti PD, PE, and PilA, Antibodies

Plates were coated for 2 hours at 37° C. with 100 μl per well of 2 μg/ml of PD (1 mg/ml), 2 μg/ml of PE (1500 μg/ml), or 2 μg/ml of PilA (3660 μg/ml) in carbonate buffer pH 9.6 in PBS or Affinipure Goat anti-guinea pig IgG (2.4 mg/ml) diluted to 2 μg/m for the reference wells in PBS. The plates were washed 4 times with NaCl 0.9% Polysorbate 20 0.05%. For PE and PilA ELISAs (this step was not carried out for the PD and Ply ELISAs), the plates were saturated 30 min at room temperature with PBS-BSA1%. After washing, sera from guinea pigs immunised as described in the section entitled ‘immunogenicity of three formulations in guinea pigs’ or a reference serum sample (an internal reference calibrated with Chrompure guinea pig IgG) was added to microwells and serially diluted 100 μl (two-fold dilution step) in PBS Polysorbate 20 0.05% (for the PD ELISA) and PBS Polysorbate 20 0.05% BSA 0.1% for the PE and PilA ELISAs. The plates were incubated for 30 min at room temperature. After washing, the plates were incubated with an anti-guinea pig IgG antibody conjugated to peroxydase (100 μl per well) at room temperature for 30 minutes with shaking. Plates were washed as above and the substrate conjugate (4 mg of OPDA in 10 ml of citrate 0.1M pH 4.5-4.6 and 5 μl of H₂O₂) was added to each well (100 μl) for 15 min in darkness. The reaction was stopped by addition of HCl 1N 50 μl and the absorbance is read at 490 nm (620 nm for the reference filter).

Description of the ELISA to Measure PhtD and dPly Antibodies

Plates were coated for 2 hours at 37° C. with 100 μl per well of 1 μg/ml of PhtD (1021 μg/ml) or 2 μg/ml Ply (376 μg/ml) in PBS. The plates were then washed 4 times with NaCl 0.9% Polysorbate 20 0.05%. After washing, sera from guinea pigs immunised as described in the section entitled ‘immunogenicity of three vaccine formulations in guinea pigs’ or a reference (an internal reference calibrated with Chrompure guinea pig IgG) was added to microwells and serially diluted 100 μl (two-fold dilution step) in PBS Polysorbate 20 0.05%. The plates were incubated for 30 min at room temperature with agitation. After washing, the plates were incubated with an anti-guinea pig IgG antibody conjugated to peroxydase (100 μl per well) at room temperature for 30 minutes with shaking. Plates were washed as above and the substrate conjugate (4 mg of OPDA in 10 ml of citrate 0.1M pH 4.5-4.6 and 5 μl of H₂O₂) was added to each well (100 μl) for 15 min in darkness. The reaction was stopped by addition of HCl 1N 50 μl and the absorbance was read at 490 nm (620 nm for the reference filter).

Opsonophagocytosis Assay

Serum samples were heated for 45 min at 56° C. to inactivate any remaining endogenous complement. Twenty-five microlitres aliquots of each 1:2 diluted serum sample was two-fold serially diluted in 25 μl OPA buffer (HBSS (Hank's Balanced Salt Solution)-14.4% inactivated FCS (foetal calf serum)) per well of a 96-well round bottom microtitre plate. Subsequently, 25 μl of a mixture of activated HL-60 cells (1×107 cells/ml), freshly thawed pneumococcal working seed and freshly thawed baby rabbit complement in an e.g. 4/2/1 ratio (v/v/v) (except for serotypes 1, 6B and 6A for which the ratio was 4/2/2) was added to the diluted sera to yield a final volume of 50 μl. The assay plate was incubated for 2 h at 37° C. with orbital shaking (210 rpm) to promote the phagocytic process. The reaction was stopped by laying the microplate on ice for at least 1 min (the plate should be kept on ice until further use. A 20 μl aliquot of each well of the plate was then transferred into the corresponding well of a 96-well flat bottom microplate and 50 μl of Todd-Hewitt Broth-0.9% agar was added to each well. After overnight incubation at 37° C. and 5% CO2, pneumococcal colonies appearing in the agar were counted using an automated image analysis system (KS 400, Zeiss, Oberkochen, Germany). Eight wells without serum sample were used as bacterial controls to determine the number of pneumococci per well. The mean number of CFU of the control wells was determined and used for the calculation of the killing activity for each serum sample. The OPA titre for the serum samples was determined by the reciprocal dilution of serum able to facilitate 50% killing of the pneumococci. The opsonophagocytic titre was calculated by using a 4-parameter curve fit analysis.

Immunogenicity of Three Formulations in Guinea Pigs

2 groups of 17 guinea pigs were immunized by intramuscular (IM) injections on days 0, 14 and 28 with ¼ human dose of different formulation including Proteins alone (PhtD, dPly and PEPilA—results not presented), Prevnar 13 (™ a commercially available Streptococcal vaccine—results not presented) 10V, 12V (DSP2A017) and 12V+proteins (DSP2A012) GMP lots. Guinea pigs received in a different leg (mimicking co-administration at different sites in infants in clinical trials) ¼ of human dose of Infanrix Hexa (™ a vaccine comprising diphtheria toxoid, tetanus toxoid, pertussis toxoid, filamentous haemmagglutinin, pertactin, hepatitis B surface antigen, inactivated polio virus types 1, 2 and 3 and Haemophilus influenzae b saccharide (PRP).

Anti-IgG levels and opsonophagocytosis titers were determined respectively in individual and pooled sera collected at days 42.

The IgG antibody titers and opsonic activity was evaluated and compared between the 12V+proteins, 12V and 10V vaccines.

Sera from guinea pigs injected with the different formulations were tested in ELISA against the polysaccharide serotypes and proteins and in OPA against the 12 serotypes in the formulation.

The 12V+proteins induced similar responses to the 12V formulation.

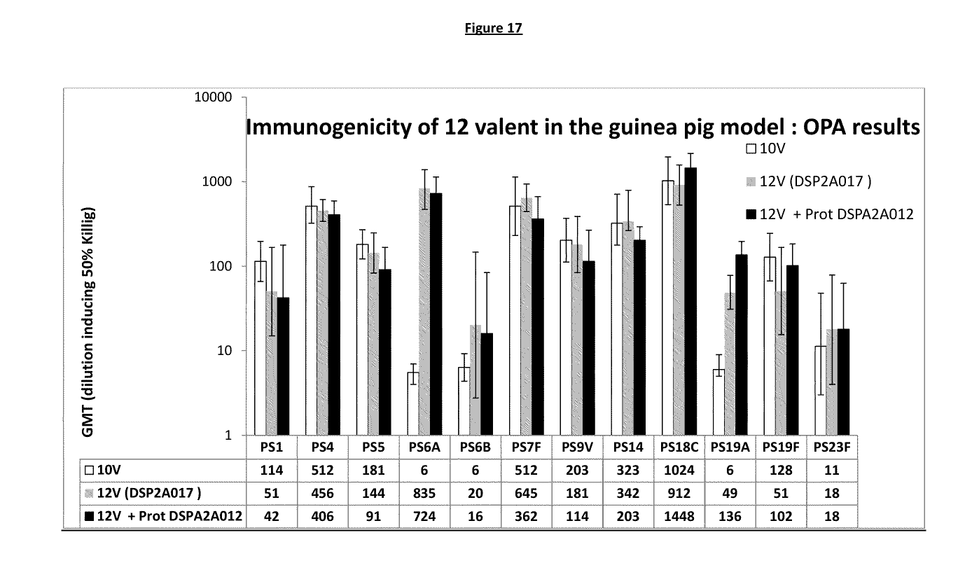

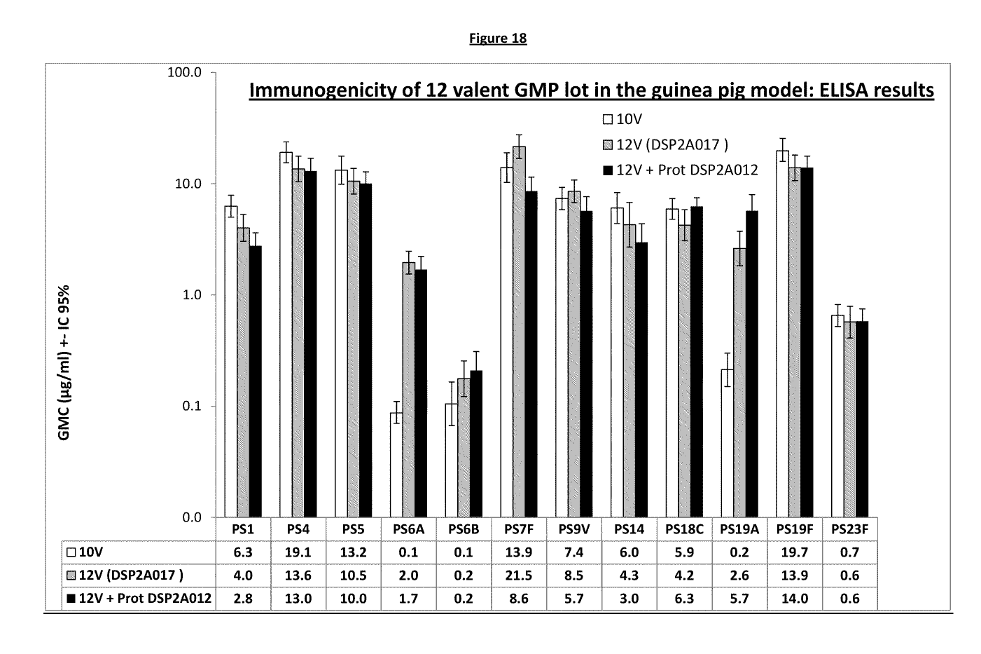

The results in FIGS. 17 and 18 demonstrated that there is no negative impact on the immunogenicity of the 12 valent conjugates when they are combined with PEPilA.

The results obtained with the 12V+proteins GMP formulation demonstrated immunogenicity of the 10V polysaccharides as well as the proteins and support the clinical evaluation of this formulation.

1. An immunogenic composition for enhancing antibody-mediated opsonic activity against a targeted serotype of Streptococcus pneumoniae in a human subject comprising
(i) at least one Streptococcus pneumoniae protein; and
(ii) a Streptococcus pneumoniae capsular saccharide derived from a strain of the targeted serotype of Streptococcus pneumoniae.

2. An immunogenic composition for immunising a human subject against Streptococcus pneumoniae comprising
(i) at least one Streptococcus pneumoniae protein; and
(ii) a Streptococcus pneumoniae capsular saccharide derived from a strain of a targeted serotype of Streptococcus pneumoniae;

3-8. (canceled)

9. A method for enhancing antibody-mediated opsonic activity against a targeted serotype of Streptococcus pneumoniae in a subject the method comprising:
co-administering (i) at least one Streptococcus pneumoniae protein; and (ii) a Streptococcus pneumoniae capsular saccharide derived from a strain of the targeted serotype of Streptococcus pneumoniae; wherein co-administering the Streptococcus pneumoniae capsular saccharide in combination with the at least one Streptococcus pneumoniae protein enhances antibody-mediated opsonic activity against the targeted serotype of Streptococcus pneumoniae;
wherein co-administering is performed by administering an immunogenic composition comprising the Streptococcus pneumoniae capsular saccharide in combination with the at least one Streptococcus pneumoniae protein.

10. A method of immunising a human host against diseases caused by Streptococcus pneumoniae infection comprising administering to the host an immunoprotective dose of an immunogenic composition comprising (i) at least one Streptococcus pneumoniae protein; and (ii) a Streptococcus pneumoniae capsular saccharide derived from a strain of a targeted serotype of Streptococcus pneumoniae;
wherein the at least one Streptococcus pneumoniae protein enhances antibody-mediated opsonic activity against the targeted serotype of Streptococcus pneumoniae.

11. A method of increasing an immune response in a subject against infection or disease caused by Streptococcus pneumoniae, the method comprising:
co-administering (i) at least one Streptococcus pneumoniae protein; and (ii) a Streptococcus pneumoniae capsular saccharide derived from a strain of a targeted serotype of Streptococcus pneumoniae as an immunogenic composition, wherein co-administering the Streptococcus pneumoniae capsular saccharide in combination with the at least one Streptococcus pneumoniae protein enhances antibody-mediated opsonic activity against the targeted serotype of Streptococcus pneumoniae or
wherein an increased immune response is measurable as an enhanced antibody-mediated opsonic activity against the targeted serotype of Streptococcus pneumoniae.

12. A method of increasing an immune response in a subject at risk of infection with Streptococcus pneumoniae against infection or disease caused by Streptococcus pneumoniae, the method comprising:
providing at least one Streptococcus pneumoniae protein;
formulating the at least one Streptococcus pneumoniae protein in combination with a Streptococcus pneumoniae capsular saccharide derived from a strain of a targeted serotype of Streptococcus pneumoniae to produce an immunogenic composition; and
administering to the subject at risk of infection with Streptococcus pneumoniae one or more doses of the immunogenic composition, thereby eliciting an immune response, which immune response is measurable as an enhanced antibody-mediated opsonic activity against the targeted serotype of Streptococcus pneumoniae.

13. (canceled)

14. The method of any one of claims 9-12 wherein the subject or human host is selected from the group consisting of an immunocompromised human, a poor responding to immunisation with polysaccharide, an infant, a toddler and an adult.

15-18. (canceled)

19. The method of claim 14 wherein the immunocompromised human is a human who has been diagnosed with HIV infection, chronic renal failure, nephritic syndrome, a disease associated with treatment with immunosuppressive drugs or radiation therapy, or congenital immunodeficiency.

20-24. (canceled)

25. The method of claim 12 wherein the antibody-mediated opsonic activity against the targeted serotype of Streptococcus pneumoniae is enhanced if the antibody-mediated opsonic activity against the targeted strain of Streptococcus pneumoniae is greater when the Streptococcus pneumoniae capsular saccharide is co-administered with the Streptococcus pneumoniae protein than antibody-mediated opsonic activity elicited by administering an equivalent dose of the Streptococcus pneumoniae capsular saccharide alone in the absence of the at least one Streptococcus pneumoniae protein.

26. (canceled)

27. The method of claim 12 wherein the at least one Streptococcus pneumoniae protein comprises detoxified pneumolysin (dPly).

28. The method of claim 27 wherein the detoxified pneumolysin has been chemically detoxified.

29. (canceled)

30. The method of any one of claims 28-29 wherein the immunogenic composition comprises 26 μg-45 μg (for example 26 μg-40 μg, 28 μg-35 μg or around 30 μg) of pneumolysin, per human dose.

31. The method of claim 12 wherein the at least one Streptococcus pneumoniae protein comprises PhtD (Poly Histidine Triad D).

32. The method of claim 31 wherein the PhtD comprises an amino acid sequence at least 85%, 90%, 95%, 98%, 99% or 100% identical to the sequence at amino acids 21-838 of Sequence ID No. 4 of WO00/37105.

33. The method of claim 31 or 32 wherein the immunogenic composition comprises 26 μg-45 μg (for example 26 μg-40 μg, 28 μg-35 μg or around 30 μg) of PhtD, per human dose.

34-37. (canceled)

38. The method of claim 12 wherein the Streptococcus pneumoniae capsular saccharide is a capsular saccharide from a serotype selected from the group consisting of 1, 2, 3, 4, 5, 6A, 6B, 7F, 8, 9N, 9V, 10A, 11A, 12F, 15, 16B, 17F, 18C, 19A, 19F, 20, 22F, 23F and 33F.

39-43. (canceled)

44. The method of claim 38 wherein the Streptococcus pneumoniae capsular saccharide is a capsular saccharide from serotype 19F.

45-50. (canceled)

51. The method of claim 12 wherein the immunogenic composition comprises 1 or more further Streptococcus pneumoniae saccharide conjugates and wherein the 1 or more further Streptococcus pneumoniae saccharide conjugates comprises a conjugated serotype 4 saccharide, optionally wherein the immunogenic composition enhances antibody-mediated opsonic activity against serotype 4 Streptococcus pneumoniae; a conjugated serotype 6B saccharide, optionally wherein the immunogenic composition enhances antibody-mediated opsonic activity against serotype 6B Streptococcus pneumoniae; a conjugated serotype 9V saccharide, optionally wherein the immunogenic composition enhances antibody-mediated opsonic activity against serotype 9V Streptococcus pneumoniae; a conjugated serotype 14 saccharide, optionally wherein the immunogenic composition enhances antibody-mediated opsonic activity against serotype 14 Streptococcus pneumoniae; a conjugated serotype 18C saccharide, optionally wherein the immunogenic composition enhances antibody-mediated opsonic activity against serotype 18C Streptococcus pneumoniae; and a conjugated serotype 23F saccharide, optionally wherein the immunogenic composition enhances antibody-mediated opsonic activity against serotype 23F Streptococcus pneumoniae.

52-61. (canceled)

62. The method of claim 51 wherein the 1 or more further Streptococcus pneumoniae saccharide conjugates comprises a conjugated serotype 1 saccharide, optionally wherein the immunogenic composition enhances antibody-mediated opsonic activity against serotype 1 Streptococcus pneumoniae; a conjugated serotype 5 saccharide, optionally wherein the immunogenic composition enhances antibody-mediated opsonic activity against serotype 5Streptococcus pneumoniae; and a conjugated serotype 7F saccharide, optionally wherein the immunogenic composition enhances antibody-mediated opsonic activity against serotype 7F Streptococcus pneumoniae.

63-70. (canceled)

71. The method of any one of claim 51 or 62 wherein the 1 or more further Streptococcus pneumoniae saccharide conjugates are independently conjugated to a carrier protein selected from the group consisting of tetanus toxoid (TT), fragment C of TT, diphtheria toxoid, CRM197 (Cross Reacting Material 197), Pneumolysin, protein D (from Haemophilus influenzae), PhtD (Poly Histidine Triad D), PhtDE (fusion protein between Pneumococcal Hisitidine Triad D and Poly Histidine Triad E) and N19.

72-86. (canceled)

87. The method of claim 71, wherein the immunogenic composition comprises a serotype 1 saccharide conjugated to protein D, a serotype 4 saccharide conjugated to protein D, a serotype 5 saccharide conjugated to protein D, a serotype 6B saccharide conjugated to protein D, a serotype 7F saccharide conjugated to protein D, a serotype 9V saccharide conjugated to protein D a serotype 14 saccharide conjugated to protein D, a serotype 23F saccharide conjugated to protein D, a serotype 18C saccharide conjugated to tetanus toxoid and a serotype 19F saccharide conjugated to diphtheria toxoid.

88-100. (canceled)
